(19) United States
(12) Patent Application Publication
(10) Pub. No.: US 2024/0083991 A1
(43) Pub. Date: Mar. 14, 2024

(54) ANTI-BMP9 ANTIBODIES AND METHODS OF USE THEREOF

(71) Applicants: Pfizer Inc., New York, NY (US); The Brigham and Women's Hospital, Inc., Boston, MA (US); The General Hospital Corporation, Boston, MA (US)

(72) Inventors: Paul B. Yu, Boston, MA (US); James Reasoner Apgar, Newton, MA (US); Susan Adam Benard, Melrose, MA (US); Stephen Peter Berasi, Arlington, MA (US); Christine Huard, Somerville, MA (US); Oleg Victorovich Kovalenko, Arlington, MA (US); Lidia Mosyak, Newton, MA (US); Xianchun Tang, Boston, MA (US); Kathleen Elisabeth Tumelty, Brookline, MA (US); Luca Troncone, Boston, MA (US); Ying Zhong, Malden, MA (US)

(73) Assignees: Pfizer Inc., New York, NY (US); The Brigham and Women's Hospital, Inc., Boston, MA (US); The General Hospital Corporation, Boston, MA (US)

(21) Appl. No.: 18/326,335

(22) Filed: May 31, 2023

Related U.S. Application Data
(60) Provisional application No. 63/347,543, filed on May 31, 2022.
(60) Provisional application No. 63/375,781, filed on Sep. 15, 2022.
(60) Provisional application No. 63/499,808, filed on May 3, 2023.

(51) Int. Cl. C07K 16/22; A61P 9/12

(52) U.S. Cl. CPC C07K 16/22; A61P 9/12; A61K 2039/505; C07K 2317/34; C07K 2317/76; C07K 2317/92

(57) ABSTRACT

Antibodies, and antigen-binding fragments thereof, that specifically bind to bone morphogenetic protein-9 (BMP9) are provided. Embodiments include uses, and associated methods of using the antibodies, and antigen-binding fragments thereof.

### CROSS-REFERENCE TO RELATED APPLICATIONS

This application claims priority to and the benefit of U.S. Provisional Application No. 63/347,543, filed May 31, 2022, U.S. Provisional Application No. 63/375,781, filed Sep. 15, 2022, and U.S. Provisional Application No. 63/499,808, filed May 3, 2023, the contents of which applications are incorporated herein by reference.

### PARTIES TO A JOINT RESEARCH STATEMENT

The presently claimed invention was made by or on behalf of the below listed parties to a joint research agreement. The joint research agreement was in effect on or before the date the claimed invention was made, and the claimed invention was made as a result of activities undertaken within the scope of the joint research agreement. The parties to the joint research agreement are PARTNERS HEALTHCARE and PFIZER INC.

### REFERENCE TO AN ELECTRONIC SEQUENCE LISTING

The contents of the electronic sequence listing (PFZRP004 IWO Sequence Listing.xml; Size: 87,500 bytes; and Date of Creation: Sep. 15, 2022) is herein incorporated by reference in its entirety.

### BACKGROUND

Pulmonary arterial hypertension (PAH) is a rare, progressive disorder characterized by high blood pressure (hypertension) in the arteries of the lungs. PAH is defined as a mean pulmonary artery pressure (mPAP) greater than 20 mmHg in the setting of normal or reduced cardiac output and a normal pulmonary capillary wedge pressure (Simonneau G, Hoeper M M, The revised definition of pulmonary hypertension: exploring the impact on patient management. European Heart Journal Supplements, 2019; 21: K4-K8). Initial symptoms of PAH include shortness of breath following exertion. Additional symptoms include excessive fatigue, weakness, chest pain, dizzy spells, and fainting episodes. In advanced stages of PAH, individuals may have abnormal bluish discoloration of the skin due to low levels of circulating oxygen (cyanosis) and the right chamber (ventricle) of the heart may be abnormally enlarged (hypertrophy), potentially resulting in heart failure.

Current methods for treating PAH include vasodilators, anti-proliferative agents, calcium channel blockers, blood thinners, and diuretics. These therapies focus on prolonging the lifespan of the patient and improving quality of life. However, these treatments have one or more disadvantages, such as lack of effectiveness, serious side effects, low patient compliance, and high cost. In fact, most patients still die from the diseases or fail to respond adequately to medical therapy with a 5-year survival rate of 59% (Boucly A, Weatherald J, Savale L, et al. Risk assessment, prognosis, and guideline implementation in pulmonary arterial hypertension. Eur Respir J. 2017; 50:1700889). Accordingly, a significant need exists in the art for novel targeted therapies for the treatment and/or prevention of PAH and, in particular, to address the underlying pathophysiology.

### SUMMARY

The present disclosure provides antibodies that bind to bone morphogenetic protein-9 (BMP9), encoded by the growth differentiation factor 2 (GDF2) gene, as well as uses of the antibodies and associated methods. Polynucleotides encoding antibodies that bind BMP9 are provided. Polynucleotides encoding antibody heavy chains or light chains, or both, are also provided. Host cells that express the antibodies are provided. The disclosure also provides processes for making, preparing, and producing antibodies that bind to BMP9. The disclosure further encompasses expression of antibodies, and preparation and manufacture of compositions comprising antibodies of the disclosure, such as medicaments for the use of the antibodies.

Antibodies of the disclosure are useful in one or more of diagnosis, prophylaxis, and/or treatment of disorders or conditions mediated by, or associated with, BMP9 activity, including, but not limited to, pulmonary arterial hypertension, anemias, liver diseases, neuroinflammatory or neurodegenerative diseases such as multiple sclerosis, fibrotic disorders, and heart failure. Any one or more of the foregoing disorders or conditions may be excluded from diagnosis, prophylaxis, and/or treatment mediated by, or associated with, BMP9 activity.

Disclosed herein, in some aspects, is an isolated antibody that binds to bone morphogenetic protein-9 (BMP9) and comprises a heavy chain variable region (VH) and a light chain variable region (VL). In some aspects, the VH complementarity determining region one (CDR1) comprises an amino acid sequence selected from the group consisting of SEQ ID NO: 9, SEQ ID NO: 24, SEQ ID NO: 36, SEQ ID NO: 45, and SEQ ID NO: 53, the VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76, the VH CDR3 comprises an amino acid sequence selected from the group consisting of SEQ ID NO: 11, SEQ ID NO: 26, SEQ ID NO: 38, SEQ ID NO: 47, and SEQ ID NO: 62, the VL CDR1 comprises an amino acid sequence selected from the group consisting of SEQ ID NO: 2, SEQ ID NO: 19, SEQ ID NO: 30, SEQ ID NO: 50, SEQ ID NO: 57, SEQ ID NO: 65, and SEQ ID NO: 73, the VL CDR2 comprises an amino acid sequence selected from the group consisting of SEQ ID NO: 3, SEQ ID NO: 20, SEQ ID NO: 31, SEQ ID NO: 41, and SEQ ID NO: 66, and the VL CDR3 comprises an amino acid sequence selected from the group consisting of SEQ ID NO: 4, SEQ ID NO: 21, SEQ ID NO: 32, SEQ ID NO: 42, SEQ ID NO: 58, and SEQ ID NO: 67.

Disclosed herein, in some aspects, is an isolated antibody that binds to bone morphogenetic protein-9 (BMP9) and comprises a heavy chain variable region (VH) and a light chain variable region (VL), wherein (i) the VH CDR1 comprises the amino acid sequence of SEQ ID NO: 80, the VH CDR2 comprises the amino acid sequence of SEQ ID NO: 81, and the VH CDR3 comprises the amino acid sequence of SEQ ID NO: 26, and the VL CDR1 comprises the amino acid sequence of SEQ ID NO: 82, the VL CDR2 comprises the amino acid sequence of SEQ ID NO: 83, and the VL CDR3 comprises the amino acid sequence of SEQ ID NO: 84; (ii) the VH CDR1 comprises the amino acid sequence of SEQ ID NO: 9, the VH CDR2 comprises the amino acid sequence of SEQ ID NO: 10, and the VH CDR3 comprises the amino acid sequence of SEQ ID NO: 11, and the VL CDR1 comprises the amino acid sequence of SEQ ID NO: 2, the VL CDR2 comprises the amino acid sequence of SEQ ID NO: 3, and the VL CDR3 comprises the amino acid sequence of SEQ ID NO: 4; (iii) the VH CDR1 comprises the amino acid sequence of SEQ ID NO: 24, the VH CDR2 comprises the amino acid sequence of SEQ ID NO: 25, and the VH CDR3 comprises the amino acid sequence of SEQ ID NO: 26, and the VL CDR1 comprises the amino acid sequence of SEQ ID NO: 19, the VL CDR2 comprises the amino acid sequence of SEQ ID NO: 20, and the VL CDR3 comprises the amino acid sequence of SEQ ID NO: 21; (iv) the VH CDR1 comprises the amino acid sequence of SEQ ID NO: 36, the VH CDR2 comprises the amino acid sequence of SEQ ID NO: 37, and the VH CDR3 comprises the amino acid sequence of SEQ ID NO: 38, and the VL CDR1 comprises the amino acid sequence of SEQ ID NO: 30, the VL CDR2 comprises the amino acid sequence of SEQ ID NO: 31, and the VL CDR3 comprises the amino acid sequence of SEQ ID NO: 32; (v) the VH CDR1 comprises the amino acid sequence of SEQ ID NO: 45, the VH CDR2 comprises the amino acid sequence of SEQ ID NO: 46, and the VH CDR3 comprises the amino acid sequence of SEQ ID NO: 47, and the VL CDR1 comprises the amino acid sequence of SEQ ID NO: 2, the VL CDR2 comprises the amino acid sequence of SEQ ID NO: 41, and the VL CDR3 comprises the amino acid sequence of SEQ ID NO: 42; (vi) the VH CDR1 comprises the amino acid sequence of SEQ ID NO: 53, the VH CDR2 comprises the amino acid sequence of SEQ ID NO: 54, and the VH CDR3 comprises the amino acid sequence of SEQ ID NO: 26, and the VL CDR1 comprises the amino acid sequence of SEQ ID NO: 50, the VL CDR2 comprises the amino acid sequence of SEQ ID NO: 20, and the VL CDR3 comprises the amino acid sequence of SEQ ID NO: 21; (vii) the VH CDR1 comprises the amino acid sequence of SEQ ID NO: 45, the VH CDR2 comprises the amino acid sequence of SEQ ID NO: 61, and the VH CDR3 comprises the amino acid sequence of SEQ ID NO: 62, and the VL CDR1 comprises the amino acid sequence of SEQ ID NO: 57, the VL CDR2 comprises the amino acid sequence of SEQ ID NO: 41, and the VL CDR3 comprises the amino acid sequence of SEQ ID NO: 58; (viii) the VH CDR1 comprises the amino acid sequence of SEQ ID NO: 53, the VH CDR2 comprises the amino acid sequence of SEQ ID NO: 70, and the VH CDR3 comprises the amino acid sequence of SEQ ID NO: 26 and the VL CDR1 comprises the amino acid sequence of SEQ ID NO: 65, the VL CDR2 comprises the amino acid sequence of SEQ ID NO: 66, and the VL CDR3 comprises the amino acid sequence of SEQ ID NO: 67; or (ix) the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.

In some embodiments of the isolated antibodies disclosed herein: (i) the VH CDR1 comprises the amino acid sequence of SEQ ID NO: 80, the VH CDR2 comprises the amino acid sequence of SEQ ID NO: 81, and the VH CDR3 comprises the amino acid sequence of SEQ ID NO: 26, and the VL CDR1 comprises the amino acid sequence of SEQ ID NO: 82, the VL CDR2 comprises the amino acid sequence of SEQ ID NO: 83, and the VL CDR3 comprises the amino acid sequence of SEQ ID NO: 84; (ii) the VH CDR1 comprises the amino acid sequence of SEQ ID NO: 24, the VH CDR2 comprises the amino acid sequence of SEQ ID NO: 25, and the VH CDR3 comprises the amino acid sequence of SEQ ID NO: 26, and the VL CDR1 comprises the amino acid sequence of SEQ ID NO: 19, the VL CDR2 comprises the amino acid sequence of SEQ ID NO: 20, and the VL CDR3 comprises the amino acid sequence of SEQ ID NO: 21; (iii) the VH CDR1 comprises the amino acid sequence of SEQ ID NO: 53, the VH CDR2 comprises the amino acid sequence of SEQ ID NO: 54, and the VH CDR3 comprises the amino acid sequence of SEQ ID NO: 26, and the VL CDR1 comprises the amino acid sequence of SEQ ID NO: 50, the VL CDR2 comprises the amino acid sequence of SEQ ID NO: 20, and the VL CDR3 comprises the amino acid sequence of SEQ ID NO: 21; (iv) the VH CDR1 comprises the amino acid sequence of SEQ ID NO: 53, the VH CDR2 comprises the amino acid sequence of SEQ ID NO: 70, and the VH CDR3 comprises the amino acid sequence of SEQ ID NO: 26, and the VL CDR1 comprises the amino acid sequence of SEQ ID NO: 65, the VL CDR2 comprises the amino acid sequence of SEQ ID NO: 66, and the VL CDR3 comprises the amino acid sequence of SEQ ID NO: 67; or (v) the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.

Disclosed herein, in some aspects, is an isolated antibody that binds to bone morphogenetic protein-9 (BMP9) and comprises a heavy chain variable region (VH) and a light chain variable region (VL), wherein the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.

In some aspects, the VH of the isolated antibodies disclosed herein comprises the amino acid sequence of SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, or a variant thereof comprising one to four amino acid substitutions at residues that are not within a CDR. In some aspects, the VL of the isolated antibodies disclosed herein comprises the amino acid sequence of SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74, or a variant thereof comprising one to four amino acid substitutions at residues that are not within a CDR. In some aspects, the VH of the isolated antibodies disclosed herein comprises the amino acid sequence of SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, or a variant thereof comprising one to four amino acid substitutions at residues that are not within a CDR, and the VL of the isolated antibodies disclosed herein comprises the amino acid sequence of SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74, or a variant thereof comprising one to four amino acid substitutions at residues that are not within a CDR.

In some aspects, the VH of the isolated antibodies disclosed herein comprises an amino acid sequence at least, at most, exactly, between (inclusive or exclusive) any two of, or about 90%, 91%, 92%, 93%, 94%, 95%, 96%, 97%, 98%, 99%, or 100%, or any range thereof, identical to SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77. In some aspects, the VH of the isolated antibodies disclosed herein comprises an amino acid sequence at least 90%, at least 91%, at least 92%, at least 93%, at least 94%, at least 95%, at least 96%, at least 97%, at least 98%, at least 99%, or 100% identical to SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77. In some aspects, the VL of the isolated antibodies disclosed herein comprises an amino acid sequence at least, at most, exactly, between (inclusive or exclusive) any two of, or about 90%, 91%, 92%, 93%, 94%, 95%, 96%, 97%, 98%, 99%, or 100%, or any range thereof, identical to SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74. In some aspects, the VL of the isolated antibodies disclosed herein comprises an amino acid sequence at least 90%, at least 91%, at least 92%, at least 93%, at least 94%, at least 95%, at least 96%, at least 97%, at least 98%, at least 99%, or 100% identical to SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74. In some aspects, the VH of the isolated antibodies disclosed herein comprises an amino acid sequence at least, at most, exactly, between (inclusive or exclusive) any two of, or about 90%, 91%, 92%, 93%, 94%, 95%, 96%, 97%, 98%, 99%, or 100%, or any range thereof, (e.g., at least 90%, at least 91%, at least 92%, at least 93%, at least 94%, at least 95%, at least 96%, at least 97%, at least 98%, at least 99%, or 100%) identical to SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, and the VL of the isolated antibodies disclosed herein comprises an amino acid sequence at least, at most, exactly, between (inclusive or exclusive) any two of, or about 90%, 91%, 92%, 93%, 94%, 95%, 96%, 97%, 98%, 99%, or 100%, or any range thereof, (e.g., at least 90%, at least 91%, at least 92%, at least 93%, at least 94%, at least 95%, at least 96%, at least 97%, at least 98%, at least 99%, or 100%) identical to SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74. It is contemplated that any one or more of these foregoing percentages may be excluded in an embodiment.

In some aspects, the isolated antibodies disclosed herein comprise: (i) a VH comprising the amino acid sequence of SEQ ID NO: 16 and a VL comprising the amino acid sequence of SEQ ID NO: 6; (ii) a VH comprising the amino acid sequence of SEQ ID NO: 27 and a VL comprising the amino acid sequence of SEQ ID NO: 22; (iii) a VH comprising the amino acid sequence of SEQ ID NO: 39 and a VL comprising the amino acid sequence of SEQ ID NO: 33; (iv) a VH comprising the amino acid sequence of SEQ ID NO: 48 and a VL comprising the amino acid sequence of SEQ ID NO: 43; (v) a VH comprising the amino acid sequence of SEQ ID NO: 55 and a VL comprising the amino acid sequence of SEQ ID NO: 51; (vi) a VH comprising the amino acid sequence of SEQ ID NO: 63 and a VL comprising the amino acid sequence of SEQ ID NO: 59; (vii) a VH comprising the amino acid sequence of SEQ ID NO: 71 and a VL comprising the amino acid sequence of SEQ ID NO: 68; or (viii) a VH comprising the amino acid sequence of SEQ ID NO: 77 and a VL comprising the amino acid sequence of SEQ ID NO: 74.

In some aspects, the isolated antibodies disclosed herein comprise: (i) a VH comprising the amino acid sequence of SEQ ID NO: 27 and a VL comprising the amino acid sequence of SEQ ID NO: 22; (ii) a VH comprising the amino acid sequence of SEQ ID NO: 55 and a VL comprising the amino acid sequence of SEQ ID NO: 51; (iii) a VH comprising the amino acid sequence of SEQ ID NO: 71 and a VL comprising the amino acid sequence of SEQ ID NO: 68; (iv) a VH comprising the amino acid sequence of SEQ ID NO: 77 and a VL comprising the amino acid sequence of SEQ ID NO: 74; (v) a VH comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 27 and a VL comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 22; (vi) a VH comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 55 and a VL comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 51; (vii) a VH comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 71 and a VL comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 68; or (viii) a VH comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 77 and a VL comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 74. It is contemplated that any one or more of these foregoing percentages may be excluded in an embodiment. In some aspects, the isolated antibody comprises a VH comprising the amino acid sequence of SEQ ID NO: 77 and a VL comprising the amino acid sequence of SEQ ID NO: 74.

Disclosed herein, in some aspects, is a portion of an isolated antibody, wherein the isolated antibody binds to BMP9 and comprises a heavy chain variable region (VH) and a light chain variable region (VL), wherein the VH CDR1 comprises an amino acid sequence selected from the group consisting of SEQ ID NO: 9, SEQ ID NO: 24, SEQ ID NO: 36, SEQ ID NO: 45, and SEQ ID NO: 53, the VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76, the VH CDR3 comprises an amino acid sequence selected from the group consisting of SEQ ID NO: 11, SEQ ID NO: 26, SEQ ID NO: 38, SEQ ID NO: 47, and SEQ ID NO: 62, the VL CDR1 comprises an amino acid sequence selected from the group consisting of SEQ ID NO: 2, SEQ ID NO: 19, SEQ ID NO: 30, SEQ ID NO: 50, SEQ ID NO: 57, SEQ ID NO: 65, and SEQ ID NO: 73, the VL CDR2 comprises an amino acid sequence selected from the group consisting of SEQ ID NO: 3, SEQ ID NO: 20, SEQ ID NO: 31, SEQ ID NO: 41, and SEQ ID NO: 66, and the VL CDR3 comprises an amino acid sequence selected from the group consisting of SEQ ID NO: 4, SEQ ID NO: 21, SEQ ID NO: 32, SEQ ID NO: 42, SEQ ID NO: 58, and SEQ ID NO: 67. In some aspects, the portion binds to BMP9.

In some aspects, the portion comprises a VH CDR1 comprising an amino acid sequence selected from the group consisting of SEQ ID NO: 9, SEQ ID NO: 24, SEQ ID NO: 36, SEQ ID NO: 45, and SEQ ID NO: 53, a VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76, and a VH CDR3 comprises an amino acid sequence selected from the group consisting of SEQ ID NO: 11, SEQ ID NO: 26, SEQ ID NO: 38, SEQ ID NO: 47, and SEQ ID NO: 62.

Disclosed herein, in some aspects, is an isolated antibody that binds to BMP9 and comprises a VH encoded by the nucleic acid sequence of the insert of the plasmid deposited with the ATCC and having the Accession Number PTA-127292, a VL encoded by the nucleic acid sequence of the insert of the plasmid deposited with the ATCC and having the Accession Number PTA-127293, or both.

Disclosed herein, in some aspects, is an isolated antibody comprising: (i) the VH CDR1, VH CDR2, and VH CDR3 sequences of SEQ ID NO: 16 and the VL CDR1, VL CDR2, and VL CDR3 sequences of SEQ ID NO: 6; (ii) the VH CDR1, VH CDR2, and VH CDR3 sequences of SEQ ID NO: 27 and the VL CDR1, VL CDR2, and VL CDR3 sequences of SEQ ID NO: 22; (iii) the VH CDR1, VH CDR2, and VH CDR3 sequences of SEQ ID NO: 39 and the VL CDR1, VL CDR2, and VL CDR3 sequences of SEQ ID NO: 33; (iv) the VH CDR1, VH CDR2, and VH CDR3 sequences of SEQ ID NO: 48 and the VL CDR1, VL CDR2, and VL CDR3 sequences of SEQ ID NO: 43; (v) the VH CDR1, VH CDR2, and VH CDR3 sequences of SEQ ID NO: 55 and the VL CDR1, VL CDR2, and VL CDR3 sequences of SEQ ID NO: 51; (vi) the VH CDR1, VH CDR2, and VH CDR3 sequences of SEQ ID NO: 63 and the VL CDR1, VL CDR2, and VL CDR3 sequences of SEQ ID NO: 59; (vii) the VH CDR1, VH CDR2, and VH CDR3 sequences of SEQ ID NO: 71 and the VL CDR1, VL CDR2, and VL CDR3 sequences of SEQ ID NO: 68; or (viii) the VH CDR1, VH CDR2, and VH CDR3 sequences of SEQ ID NO: 77 and the VL CDR1, VL CDR2, and VL CDR3 sequences of SEQ ID NO: 74.

In some aspects, the isolated antibody comprises a human V_{κ} or Vλ light chain constant domain. In some aspects, the antibody comprises a human V_{λ} light chain constant domain. In some aspects, the antibody comprises a heavy chain constant domain. In some aspects, the heavy chain constant domain comprises an IgA (for example, IgA₁ or IgA₂), IgD, IgE, IgM, or IgG (for example IgG₁, IgG₂, IgG₃, or IgG₄). In some aspects, the heavy chain constant domain comprises an IgG. In some aspects, the IgG is selected from the group consisting of IgG₁, IgG₂, IgG₃, and IgG₄. In some aspects, the IgG is IgG₁. In some aspects, the antibody comprises an Fc domain. In some aspects, the Fc domain comprises an IgG₁ heavy chain CH2 domain and an IgG heavy chain CH3 domain.

Disclosed herein, in some aspects, is an isolated antibody comprising a heavy chain comprising an amino acid sequence selected from the group consisting of SEQ ID NO: 8, SEQ ID NO: 23, SEQ ID NO: 35, SEQ ID NO: 44, SEQ ID NO: 52, SEQ ID NO: 60, SEQ ID NO: 69, and SEQ ID NO: 75. Disclosed herein, in some aspects, is an isolated antibody comprising a light chain comprising an amino acid sequence selected from the group consisting of SEQ ID NO: 1, SEQ ID NO: 18, SEQ ID No: 29, SEQ ID NO: 40, SEQ ID NO: 49, SEQ ID NO: 56, SEQ ID NO: 64, and SEQ ID NO: 72. Disclosed herein, in some aspects, is an isolated antibody comprising: (i) a heavy chain comprising an amino acid sequence selected from the group consisting of SEQ ID NO: 8, SEQ ID NO: 23, SEQ ID NO: 35, SEQ ID NO: 44, SEQ ID NO: 52, SEQ ID NO: 60, SEQ ID NO: 69, and SEQ ID NO: 75, and (ii) a light chain comprising an amino acid sequence selected from the group consisting of SEQ ID NO: 1, SEQ ID NO: 18, SEQ ID No: 29, SEQ ID NO: 40, SEQ ID NO: 49, SEQ ID NO: 56, SEQ ID NO: 64, and SEQ ID NO: 72.

Disclosed herein, in some aspects, is an isolated antibody comprising a heavy chain comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 8, SEQ ID NO: 23, SEQ ID NO: 35, SEQ ID NO: 44, SEQ ID NO: 52, SEQ ID NO: 60, SEQ ID NO: 69, or SEQ ID NO: 75. Disclosed herein, in some aspects, is an isolated antibody comprising a light chain comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 1, SEQ ID NO: 18, SEQ ID No: 29, SEQ ID NO: 40, SEQ ID NO: 49, SEQ ID NO: 56, SEQ ID NO: 64, or SEQ ID NO: 72. Disclosed herein, in some aspects, is an isolated antibody comprising: (i) a heavy chain comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 8, SEQ ID NO: 23, SEQ ID NO: 35, SEQ ID NO: 44, SEQ ID NO: 52, SEQ ID NO: 60, SEQ ID NO: 69, or SEQ ID NO: 75, and (ii) a light chain comprising an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 1, SEQ ID NO: 18, SEQ ID No: 29, SEQ ID NO: 40, SEQ ID NO: 49, SEQ ID NO: 56, SEQ ID NO: 64, or SEQ ID NO: 72. It is contemplated that any one or more of these foregoing percentages may be excluded in an embodiment.

Disclosed herein, in some aspects, is an isolated antibody comprising: (i) a HC comprising, or consisting of, the amino acid sequence of SEQ ID NO: 8 and a LC comprising, or consisting of, the amino acid sequence of SEQ ID NO: 1; (ii) a HC comprising, or consisting of, the amino acid sequence of SEQ ID NO: 23 and a LC comprising, or consisting of, the amino acid sequence of SEQ ID NO: 18; (iii) a HC comprising, or consisting of, the amino acid sequence of SEQ ID NO: 35 and a LC comprising, or consisting of, the amino acid sequence of SEQ ID NO: 29; (iv) a HC comprising, or consisting of, the amino acid sequence of SEQ ID NO: 44 and a LC comprising, or consisting of, the amino acid sequence of SEQ ID NO: 40; (v) a HC comprising, or consisting of, the amino acid sequence of SEQ ID NO: 52 and a LC comprising, or consisting of, the amino acid sequence of SEQ ID NO: 49; (vi) a HC comprising, or consisting of, the amino acid sequence of SEQ ID NO: 60 and a LC comprising, or consisting of, the amino acid sequence of SEQ ID NO: 56; (vii) a HC comprising, or consisting of, the amino acid sequence of SEQ ID NO: 69 and a LC comprising, or consisting of, the amino acid sequence of SEQ ID NO: 64; or (viii) a HC comprising, or consisting of, the amino acid sequence of SEQ ID NO: 75 and a LC comprising, or consisting of, the amino acid sequence of SEQ ID NO: 72.

Disclosed herein, in some aspects, is an isolated antibody comprising: (i) a HC comprising, or consisting of, the amino acid sequence of SEQ ID NO: 23 and a LC comprising, or consisting of, the amino acid sequence of SEQ ID NO: 18; (ii) a HC comprising, or consisting of, the amino acid sequence of SEQ ID NO: 52 and a LC comprising, or consisting of, the amino acid sequence of SEQ ID NO: 49; (iii) a HC comprising, or consisting of, the amino acid sequence of SEQ ID NO: 69 and a LC comprising, or consisting of, the amino acid sequence of SEQ ID NO: 64; (iv) a HC comprising, or consisting of, the amino acid sequence of SEQ ID NO: 75 and a LC comprising, or consisting of, the amino acid sequence of SEQ ID NO: 72; (v) a HC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 23 and a LC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 18; (vi) a HC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 52 and a LC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 49; (vii) a HC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 69 and a LC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 64; or (viii) a HC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 75 and a LC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 72. It is contemplated that any one or more of these foregoing percentages may be excluded in an embodiment.

Disclosed herein, in some aspects, is an isolated antibody comprising: (i) a HC comprising, or consisting of, the amino acid sequence of SEQ ID NO: 75 and a LC comprising, or consisting of, the amino acid sequence of SEQ ID NO: 72 or (ii) a HC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 75 and a LC comprising, or consisting of, an amino acid sequence at least, at most, exactly, between (inclusive or exclusive) any two of, or about 90%, 91%, 92%, 93%, 94%, 95%, 96%, 97%, 98%, 99%, or 100%, or any range thereof, e.g., at least 90%, at least 91%, at least 92%, at least 93%, at least 94%, at least 95%, at least 96%, at least 97%, at least 98%, at least 99%, or 100%, identical to SEQ ID NO: 72. It is contemplated that any one or more of these foregoing percentages may be excluded in an embodiment.

Disclosed herein, in some aspects, is an isolated antibody that binds to BMP9, wherein the antibody binds to an epitope on BMP9 comprising one or more amino acid residues selected from the group consisting of Ala28, Pro29, Ser83, Leu85, Met90, Val92, Pro93, Leu95, and Tyr97 of SEQ ID NO: 79. In some aspects, the antibody binds Ala28, Pro29, Ser83, Leu85, Met90, Val92, Pro93, Leu95, and Tyr97 of SEQ ID NO: 79. In some aspects, the antibody further binds one or more amino acid residues selected from the group consisting of Pro81, Tyr86, Asp88, Asp89, Thr94, Lys96, His98, Tyr99 and Glu100 of SEQ ID NO: 79. In some aspects, the antibody binds Pro81, Tyr86, Asp88, Asp89, Thr94, Lys96, His98, Tyr99, and Glu100 of SEQ ID NO: 79. In some aspects, the antibody further binds one or more amino acid residues selected from the group consisting of Ile27, Lys30, and Glu31 of SEQ ID NO: 79. In some aspects, the antibody binds Ile27, Lys30, and Glu31 of SEQ ID NO: 79. It is contemplated that any one or more of these foregoing amino acid residues may be excluded in an embodiment.

Disclosed herein, in some aspects, is an isolated antibody that binds to BMP9, wherein the antibody binds to an epitope on BMP9 comprising one or more amino acid residues selected from the group consisting of Ile27, Ala28, Pro29, Lys30, Glu31, Tyr32, Ser80, Pro81, Ile82, Ser83, Val84, Leu85, Tyr86, Asp88, Asp89, Met90, Val92, Pro93, Thr94, Leu95, Lys96, Tyr97, His98, Tyr99, and Glu100 of SEQ ID NO: 79. It is contemplated that any one or more of these foregoing amino acid residues may be excluded in an embodiment.

Disclosed herein, in some aspects, is an isolated antibody that binds to BMP9, wherein the antibody binds to an epitope on BMP9 comprising Ile27, Ala28, Pro29, Lys30, Glu31, Tyr32, Ser80, Pro81, Ile82, Ser83, Val84, Leu85, Tyr86, Asp88, Asp89, Met90, Val92, Pro93, Thr94, Leu95, Lys96, Tyr97, His98, Tyr99, and Glu100 of SEQ ID NO: 79.

Disclosed herein, in some aspects, is an isolated antibody that binds to BMP9, wherein the antibody binds to an epitope on BMP9 comprising one or more amino acid residues selected from the group consisting of Ala28, Pro29, Pro81, Ser83, Leu85, Tyr86, Asp88, Asp89, Met90, Val92, Pro93, Thr94, Leu95, Lys96, Tyr97, His98, Tyr99, and Glu100 of SEQ ID NO: 79. It is contemplated that any one or more of these foregoing amino acid residues may be excluded in an embodiment.

Disclosed herein, in some aspects, is an isolated antibody that binds to BMP9, wherein the antibody binds to an epitope on BMP9 comprising Ala28, Pro29, Pro81, Ser83, Leu85, Tyr86, Asp88, Asp89, Met90, Val92, Pro93, Thr94, Leu95, Lys96, Tyr97, His98, Tyr99, and Glu100 of SEQ ID NO: 79.

Disclosed herein, in some aspects, is an isolated antibody that binds to BMP9, wherein the antibody binds to an epitope on BMP9 comprising one or more amino acid residues selected from the group consisting of Arg14, Ile27, Ala28, Pro29, Lys30, Glu31, Ser83, Leu85, Met90, Val92, Pro93, Leu95, and Tyr97 of SEQ ID NO: 79. It is contemplated that any one or more of these foregoing amino acid residues may be excluded in an embodiment.

Disclosed herein, in some aspects, is an isolated antibody that binds to BMP9, wherein the antibody binds to an epitope on BMP9 comprising Arg14, Ile27, Ala28, Pro29, Lys30, Glu31, Ser83, Leu85, Met90, Val92, Pro93, Leu95, and Tyr97 of SEQ ID NO: 79. In some aspects, the antibody epitope on BMP9 does not comprise amino acid resides 21-25, 43-60 of SEQ ID NO: 79.

In some aspects of the isolated antibodies disclosed herein, the antibody binds human BMP9 with an affinity of at least, at most, exactly, between (inclusive or exclusive) of any two of, or about 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, or 150 pM, or any range thereof, at 37° C. as measured by surface plasmon resonance. In some aspects of the isolated antibodies disclosed herein, the antibody binds human BMP9 with an affinity of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM at 37° C. as measured by surface plasmon resonance. In some embodiments, the antibody binds human BMP9 with an affinity of about 75 pM to about 95 pM at 37° C. as measured by surface plasmon resonance. In some embodiments, the antibody binds human BMP9 with an affinity of about 85 pM at 37° C. as measured by surface plasmon resonance. In some embodiments, the antibody binds human BMP9 with an affinity of about 87 pM at 37° C. as measured by surface plasmon resonance. It is contemplated that any one or more of these foregoing binding affinities may be excluded in an embodiment.

In some aspects of the isolated antibodies disclosed herein, the antibody binds cyno BMP9 with an affinity of at least, at most, exactly, between (inclusive or exclusive) of any two of, or about 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, or 150 pM, or any range thereof, at 37° C. as measured by surface plasmon resonance. In some aspects of the isolated antibodies disclosed herein, the antibody binds cyno BMP9 with an affinity of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM at 37° C. as measured by surface plasmon resonance. In some embodiments, the antibody binds cyno BMP9 with an affinity of about 50 pM to about 100 pM at 37° C. as measured by surface plasmon resonance. In some embodiments, the antibody binds cyno BMP9 with an affinity of about 65 pM at 37° C. as measured by surface plasmon resonance. It is contemplated that any one or more of these foregoing binding affinities may be excluded in an embodiment.

In some aspects of the isolated antibodies disclosed herein, the antibody binds rat BMP9 with an affinity of at least, at most, exactly, between (inclusive or exclusive) of any two of, or about 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, or 150 pM, or any range thereof, at 37° C. as measured by surface plasmon resonance. In some aspects of the isolated antibodies disclosed herein, the antibody binds rat BMP9 with an affinity of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM at 37° C. as measured by surface plasmon resonance. In some embodiments, the antibody binds rat BMP9 with an affinity of about 75 pM to about 120 pM at 37° C. as measured by surface plasmon resonance. In some embodiments, the antibody binds rat BMP9 with an affinity of about 98 pM at 37° C. as measured by surface plasmon resonance. It is contemplated that any one or more of these foregoing binding affinities may be excluded in an embodiment.

In some aspects of the isolated antibodies disclosed herein, the antibody comprises: (i) a heavy chain variable region (VH) CDR3 of SEQ ID NO: 26 and a light chain variable region (VL) CDR3 of SEQ ID NO: 84, (ii) a heavy chain variable region (VH) CDR3 of SEQ ID NO: 26 and a light chain variable region (VL) CDR3 of SEQ ID NO: 21, or (iii) a heavy chain variable region (VH) CDR3 of SEQ ID NO: 26 and a light chain variable region (VL) CDR3 of SEQ ID NO: 67.

In some aspects of the isolated antibodies disclosed herein, the antibody (i) binds to human BMP9 and inhibits binding of human BMP9 to a BMP9 type II receptor (e.g., BMPRII, ActRIIA, and/or ActRIIB); or (ii) binds to human BMP9 and inhibits binding of human BMP9 to a BMP9 type II receptor (e.g., BMPRII, ActRIIA, and/or ActRIIB) and weakly inhibits binding of human BMP9 to a BMP9 type 1 receptor (e.g., ALK1). In some aspects of the isolated antibodies disclosed herein, the antibody binds to human BMP9 and inhibits binding of human BMP9 to human endothelial cells. In some aspects of the isolated antibodies disclosed herein, the antibody binds human BMP9 but does not bind other human transforming growth factor beta (TGFβ) superfamily ligands (e.g., GDF8, GDF11, Activin A, TGFβ1, GDF9, and BMP10).

In some aspects of the isolated antibodies disclosed herein, the antibody inhibits BMP9 activity measured by phospho-SMAD1/5/9 nuclear translocation in endothelial cells.

In some aspects, the isolated antibody inhibits human BMP9 activity with an IC₅₀ of about 0.01 nM to about 100 nM (e.g., at least, at most, exactly, between (inclusive or exclusive) of any two of, or about 10 pM, 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 160 pM, 170 pM, 180 pM, 190 pM, 200 pM, 225 pM, 250 pM, 275 pM, 300 pM, 325 pM, 350 pM, 375 pM, 400 pM, 425 pM, 450 pM, 475 pM, 500 pM, 550 pM, 600 pM, 650 pM, 700 pM, 750 pM, 800 pM, 850 pM, 900 pM, 950 pM, 1000 pM, 2000 pM, 3000 pM, 4000 pM, 5000 pM, 6000 pM, 7000 pM, 8000 pM, 9000 pM, 10000 pM, 20000 pM, 30000 pM, 40000 pM, 50000 pM, 60000 pM, 70000 pM, 80000 pM, 90000 pM, or 100000 pM, or any range thereof). In some embodiments, the isolated antibody inhibits human BMP9 activity with an IC₅₀ of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, about 145 pM about 150 pM, about 155 pM, about 160 pM, about 170 pM, about 180 pM, about 190 pM, about 200 pM, about 225 pM, about 250 pM, about 300 pM, about 350 pM, about 400 pM, about 450 pM, about 500 pM, about 600 pM, about 700 pM, about 800 pM, about 900 pM, or about 1000 pM. In some embodiments, the isolated antibody inhibits human BMP9 activity with an IC₅₀ of about 1 nM, about 2 nM, about 10 nM, about 20 nM, about 30 nM, about 40 nM, about 50 nM, about 60 nM, about 70 nM, about 80 nM, about 90 nM or about 100 nM. It is contemplated that any one or more of these foregoing IC₅₀ values may be excluded in an embodiment.

In some aspects, the isolated antibody inhibits cyno BMP9 activity with an IC₅₀ of about 15 pM to about 120 pM (e.g., at least, at most, exactly, between (inclusive or exclusive) of any two of, or about 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, or 120 pM, or any range thereof). In some aspects, the isolated antibody inhibits cyno BMP9 activity with an IC₅₀ of about 17 pM to about 117 pM, for example, but not limited to, about 17 pM, about 20 pM, about 30 pM, about 40 pM, about 50 pM, about 60 pM, about 70 pM, about 80 pM, about 90 pM about 100 pM, or about 117 pM. In some aspects, the isolated antibody inhibits rat BMP9 activity with an IC₅₀ of about 120 pM to about 300 pM, for example, but not limited to, about 120 pM, about 150 pM, about 175 pM, about 200 pM, about 225 pM, about 250 pM, about 275 pM, or about 300 pM. It is contemplated that any one or more of these foregoing IC₅₀ values may be excluded in an embodiment.

In some aspects of the isolated antibodies disclosed herein, the antibody inhibits BMP9 activity measured by phosphor-SMAD2 nuclear translocation in endothelial cells.

In some aspects, the antibody inhibits human BMP9 activity with an IC₅₀ of about 0.01 nM to about 350 nM (e.g., at least, at most, exactly, between (inclusive or exclusive) of any two of, or about 10 pM, 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 160 pM, 170 pM, 180 pM, 190 pM, 200 pM, 225 pM, 250 pM, 275 pM, 300 pM, 325 pM, or 350 pM, or any range thereof). In some embodiments, the antibody inhibits human BMP9 activity with an IC₅₀ of about 10 pM, about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, about 145 pM about 150 pM, about 155 pM, about 160 pM, about 170 pM, about 180 pM, about 190 pM, about 200 pM, about 225 pM, about 250 pM, about 300 pM, about 350 pM, about 400 pM, about 450 pM, about 500 pM, about 600 pM, about 700 pM, about 800 pM, about 900 pM, or about 1000 pM. In some embodiments, the isolated antibody inhibits human BMP9 activity with an IC₅₀ of about 1 nM, about 2 nM, about 10 nM, about 20 nM, about 30 nM, about 40 nM, about 50 nM, about 60 nM, about 70 nM, about 80 nM, about 90 nM, about 100 nM, about 150 nM, about 200 nM, about 250 nM, about 300 nM, or about 350 nM. It is contemplated that any one or more of these foregoing IC₅₀ values may be excluded in an embodiment.

In some aspects, the isolated antibody inhibits cyno BMP9 activity with an IC₅₀ of about 15 pM to about 75 pM (e.g., at least, at most, exactly, between (inclusive or exclusive) of any two of, or about 15 pM, 20 pM, 25 pM, 30 pM, 35 pM, 40 pM, 45 pM, 50 pM, 55 pM, 60 pM, 65 pM, 70 pM, or 75 pM, or any range thereof). In some aspects, the isolated antibody inhibits cyno BMP9 activity with an IC₅₀ of about 27 pM to about 42 pM, for example, but not limited to, about 27 pM, about 30 pM, about 35 pM, about 40 pM, or about 42 pM. In some aspects, the isolated antibody inhibits rat BMP9 activity with an IC₅₀ of about 450 pM to about 1 nM, for example, but not limited to, about 650 pM, about 700 pM, about 750 pM, about 800 pM, about 850 pM, about 900 pM, about 950 pM, or about 1000 pM. It is contemplated that any one or more of these foregoing IC₅₀ values may be excluded in an embodiment.

In some aspects of the isolated antibodies disclosed herein, the antibody reduces right ventricular systolic pressure in a subject by at least, at most, exactly, between (inclusive or exclusive) any two of, or about 5%, 10%, 15%, 20%, 25%, 30%, 35%, 40%, 45%, 50%, 55%, 60%, 65%, or 70%, or any range thereof, e.g., at least 5%, at least 10%, at least 15%, at least 20%, at least 25%, at least 30%, at least 35%, at least 40%, at least 45%, at least 50%, at least 55%, at least 60%, at least 65%, or at least 70%, as compared to before antibody administration. In some aspects of the isolated antibodies disclosed herein, the antibody reduces right ventricular systolic pressure in a subject by at least, at most, exactly, between (inclusive or exclusive) any two of, or about 5%, 10%, 15%, 20%, 25%, 30%, 35%, 40%, 45%, 50%, 55%, 60%, 65%, or 70%, or any range thereof, e.g., at least 5%, at least 10%, at least 15%, at least 20%, at least 25%, at least 30%, at least 35%, at least 40%, at least 45%, at least 50%, at least 55%, at least 60%, at least 65%, or at least 70%, as compared to the percentage (%) difference observed between an isotype control antibody and normoxia condition. In some aspects of the isolated antibodies disclosed herein, the antibody reduces right ventricular hypertrophy in a subject by at least, at most, exactly, between (inclusive or exclusive) any two of, or about 2%, 5%, 10%, 15%, 20%, 25%, 30%, 35%, 40%, 45%, 50%, 55%, 60%, 65%, or 70%, or any range thereof, e.g., at least 2%, at least 5%, at least 10%, at least 15%, at least 20%, at least 25%, at least 30%, at least 35%, at least 40%, at least 45%, at least 50%, at least 55%, at least 60%, at least 65%, or at least 70%, as compared to before antibody administration. In some aspects of the isolated antibodies disclosed herein, the antibody reduces right ventricular hypertrophy in a subject by at least, at most, exactly, between (inclusive or exclusive) any two of, or about 2%, 5%, 10%, 15%, 20%, 25%, 30%, 35%, 40%, 45%, 50%, 55%, 60%, 65%, or 70%, or any range thereof, e.g., at least 2%, at least 5%, at least 10%, at least 15%, at least 20%, at least 25%, at least 30%, at least 35%, at least 40%, at least 45%, at least 50%, at least 55%, at least 60%, at least 65%, or at least 70%, as compared to as compared to the percentage (%) difference observed between an isotype control antibody and normoxia condition. In some aspects, the subject is a Sugen-Hypoxia animal model of pulmonary arterial hypertension (PAH). It is contemplated that any one or more of these foregoing reduction percentages may be excluded in an embodiment.

Disclosed herein, in some aspects, is an isolated polynucleotide encoding any one of the isolated antibodies disclosed herein. Disclosed herein, in some aspects, is an isolated nucleic acid encoding the VH, VL, or both, of an antibody that binds BMP9. In some aspects, the isolated nucleic acid comprises the nucleic acid sequence of SEQ ID NO: 85, the nucleic acid sequence of SEQ ID NO: 86, or both. Disclosed herein, in some aspects, is an isolated nucleic acid encoding the heavy chain, light chain, or both, of an antibody that binds BMP9. In some aspects, the isolated nucleic acid comprises the nucleic acid sequence of SEQ ID NO: 87, the nucleic acid sequence of SEQ ID NO: 88, or both. Disclosed herein, in some aspects, is an isolated nucleic acid encoding the VH, VL, or both, of an antibody that binds to BMP9. In some aspects, the isolated nucleic acid comprises the nucleic acid sequence of the insert of the plasmid deposited with the ATCC and having the Accession Number PTA-127292, the nucleic acid sequence of the insert of the plasmid deposited with the ATCC and having the Accession Number PTA-127293, or both.

Disclosed herein, in some aspects, is a vector comprising an isolated polynucleotide disclosed herein encoding any one of the isolated antibodies disclosed herein. Disclosed herein, in some aspects, is a vector comprising an isolated nucleic acid disclosed herein encoding the VH, VL, or both, of an antibody that binds BMP9. Disclosed herein, in some aspects, is a vector comprising an isolated nucleic acid disclosed herein encoding the heavy chain, light chain, or both, of an antibody that binds BMP9.

Disclosed herein, in some aspects, is an isolated host cell comprising an isolated polynucleotide disclosed herein encoding any one of the isolated antibodies disclosed herein. Disclosed herein, in some aspects, is an isolated host cell comprising an isolated nucleic acid disclosed herein encoding the VH, VL, or both, of an antibody that binds BMP9. Disclosed herein, in some aspects, is an isolated host cell comprising an isolated nucleic acid disclosed herein encoding the heavy chain, light chain, or both, of an antibody that binds BMP9. In some aspects, the host cell is a mammalian cell or an insect cell. In some aspects, the host cell is a mammalian cell selected from the group consisting of a CHO cell, an HEK-293 cell, an NS0 cell, a PER.C6® cell, or a Sp2.0 cell.

Disclosed herein, in some aspects, is a method of producing an antibody, comprising culturing a host cell disclosed herein under conditions that result in production of the antibody. In some aspects, the method further comprises recovering the antibody.

Disclosed herein, in some aspects, is a pharmaceutical composition comprising a therapeutically effective amount of an antibody disclosed herein and a pharmaceutically acceptable carrier. In some aspects, the composition comprises 1.12 mg/mL L-histidine, 2.67 mg/mL L-histidine hydrochloride monohydrate, 85 mg/mL sucrose, 0.05 mg/mL EDTA disodium dihydrate, 0.2 mg/mL polysorbate 80 at pH 5.8. In some aspects, the composition comprises 20 mM histidine, 8.5% sucrose, and 0.02% polysorbate 80, 0.005% EDTA at pH 5.8. In some aspects, the composition comprises about 2 mg/mL, 5 mg/mL, 10 mg/mL, 15 mg/mL, 20 mg/mL, 25 mg/mL, 50 mg/mL, 75 mg/mL, 100 mg/mL, 125 mg/mL, 150 mg/mL, 175 mg/mL, or 200 mg/mL antibody. In some aspects, the composition is suitable for subcutaneous (SC) and/or intravenous (IV) administration. In some aspects, the composition is suitable for intravenous (IV) administration. In some aspects, the composition is suitable for subcutaneous (SC) administration.

Disclosed herein, in some aspects, is a method for reducing the expression or activity of BMP9 in a subject, the method comprising administering to a subject in need thereof a therapeutically effective amount of a pharmaceutical composition disclosed herein or an antibody disclosed herein.

Disclosed herein, in some aspects, is a method of treating hypertension in a subject, the method comprising administering to a subject in need thereof a therapeutically effective amount of a pharmaceutical composition disclosed herein or an antibody disclosed herein. In some aspects, the hypertension is pulmonary hypertension.

Disclosed herein, in some aspects, is a method of treating pulmonary arterial hypertension (PAH) in a subject, the method comprising administering to a subject in need thereof a therapeutically effective amount of a pharmaceutical composition disclosed herein or an antibody disclosed herein. In some aspects, the antibody is administered in combination with a therapeutically effective amount of one or more additional therapeutically active compounds or treatment modalities effective in treating at least one sign and/or symptom of hypertension, optionally, pulmonary arterial hypertension.

Disclosed herein, in some aspects, is an isolated antibody disclosed herein, or a pharmaceutical composition disclosed herein, for use as a medicament.

Disclosed herein, in some aspects, is an isolated antibody disclosed herein, or a pharmaceutical composition disclosed herein, for use in the treatment of at least one sign and/or symptom of hypertension, optionally, pulmonary arterial hypertension.

Disclosed herein, in some aspects, is use of an isolated antibody disclosed herein, or a pharmaceutical composition disclosed herein in the manufacture of a medicament for treating at least one sign and/or symptom of hypertension, optionally, pulmonary arterial hypertension.

It is contemplated that any aspect discussed in this specification may be implemented with respect to any method or composition of the disclosure, and vice versa. Furthermore, compositions of the disclosure may be used to achieve methods of the disclosure.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect. Use of the one or more compositions may be employed based on any of the methods described herein.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

FIG. 1 depicts an exemplary graph showing the binding of antibodies to human BMP9 in an enzyme-linked immunosorbent assay (ELISA).

FIGS. 2A-2F depict exemplary competition ELISA data with BMP9 antibodies and receptor proteins ActRIIA (FIG. 2A), BMPRII (FIGS. 2B, 2E), ActRIIB (FIG. 2C), and ALK1 (FIG. 2D, 2F). Antibodies Ab89, Ab93, Ab100, and Ab101 are parental anti-BMP9 antibodies identified from a human antibody phage library selection campaign. Ab352, Ab804, and Ab1076 are optimized variants of Ab93. BM01 (referred to as AM0100 in WO 2016/193872) and BM02 (referred to as AM4405 in WO 2016/193872) were used as reference antibodies.

FIG. 3 depicts an exemplary graph showing the effect of anti-BMP9 antibodies on BMP9 binding to human telomerase-immortalized microvascular endothelial (TIME) cells.

FIGS. 4A-4C depict representative BIACORE™ sensorgrams of Ab1076 Fab binding to human (FIG. 4A), cyno (FIG. 4B), and rat (FIG. 4C) BMP9.

FIG. 5 depicts exemplary Octet sensorgrams of Ab1076 Fab binding to human Activin A, BMP10, GDF8, GDF9, GDF11, TGFβ1, and BMP9 proteins, at 37° C.

FIGS. 6A-6K depict exemplary data showing the inhibition of BMP9-induced phospho-SMAD1/5/9 (FIGS. 6A, 6C, 6E, 6G, 6I, and 6K) and phospho-SMAD2 (FIGS. 6B, 6D, 6F, 6H, and 6J) nuclear translocation by anti-BMP9 antibodies.

FIGS. 7A-7B depict exemplary graphs showing the right ventricular systolic pressure (RVSP; FIG. 7A) and right ventricular hypertrophy (RV/(LV+S); FIG. 7B) in rats treated with isotype control mAb (Iso Ctrl), ActRIIA-Fc, Ab732, or Ab1076. Data represent mean±SEM. One-way ANOVA with Tukey's multiple comparisons test performed for both RVSP and RV/(LV+S) relative to Iso Ctrl. *=p≤0.05; **=p≤0.01; and ****=p≤0.0001.

FIGS. 8A-8B depict exemplary data showing quantitative determination of total circulating BMP9 (FIG. 8A) and BMP10 (FIG. 8B) concentrations in serum from rats treated with isotype control mAb (Iso Ctrl), ActRIIA-Fc, Ab732, or Ab1076. Data represent mean±SD. One-way ANOVA with Tukey's multiple comparisons test performed for all treatment comparisons for both BMP9 and BMP10 circulating concentrations. *=p≤0.05; **=p≤0.01; ***=p≤0.001; ****=p≤0.0001.

FIGS. 9A-9B depict exemplary data showing the BMP9 binding epitopes for type II receptor ActRIIB (FIG. 9A) and the overlapping epitope for Ab1076 (FIG. 9B). The binding epitopes were calculated using the structure-based descriptor method described in the examples section below. Here, the structure of BMP9 is shown in white with the ActRIIB epitope (FIG. 9A) shown in black and the Ab1076 epitope (FIG. 9B) shown in gray overlayed over the non-binding ActRIIB epitope in black. The calculation for the ActRIIB epitope was done on the BMP9:ActRIIB complex in Protein Data Bank entry 4fao, chains A (BMP9) and E (ActRIIB). The calculation of the Ab1076 epitope was done using the Ab1076 structure, chains H+L (Fab) and G (BMP9).

FIGS. 10A-10D depict exemplary data showing the binding interface of BMP9 with Ab1076. FIG. 10A depicts the ribbon diagram of the interaction of BMP9 dimer (black) with the Fab of Ab1076 (VH: Dark gray, VL: Light Gray) as seen in the crystal structure of their complex. There is a 2-Fab:2-BMP9 ratio seen in the interaction. Important mutations shown in FIG. 10B, FIG. 10C, and FIG. 10D contribute to the ˜20× increase in binding affinity from Ab93 to Ab1076. These include the D_H53_E mutation, predicted to introduce a more optimal salt-bridge geometry with BMP9_K96; the D_L31_F mutation, predicted to increase the packing with BMP9_P29 and BMP9_Y32; and the V_L93_E mutation, predicted to add an additional hydrogen bond to BMP9_S80.

FIGS. 11A-11B depict exemplary data showing serum-mediated modulation of phospho-SMAD1/5/9 (FIG. 11A) and phospho-SMAD2 (FIG. 11B) nuclear translocation induced in human endothelial cells by serum from rats treated with isotype control (Iso Ctrl), ActRIIA-Fc, Ab732, or Ab1076. Data represent mean±SD. One-way ANOVA with Tukey's multiple comparisons test performed for both phospho-SMAD1/5/9 and phospho-SMAD2 relative to Iso Ctrl. *=p≤0.05; **=p≤0.01; ***=p≤0.001, and ****=p≤0.0001.

FIGS. 12A-12B depict exemplary graphs showing effects of anti-BMP9 antibodies Ab732 and Ab1076 on GDF8- and GDF11-induced luciferase activity.

FIG. 13 depicts an exemplary graph showing inhibition of endothelial secretion of endothelin-1 by anti-BMP9 antibody Ab1076 in enzyme-linked immunosorbent assay (ELISA).

FIGS. 14A-14F depict differential expression analyses for determining the relative mRNA expression level (fold change) for Cxcl12 (FIG. 14A), Igfbp4 (FIG. 14B), Inhba (FIG. 14C), Mall (FIG. 14D), Frzb (FIG. 14E), and Cpe (FIG. 14F) in SU5416+Hypoxia-treated (SuHx) rat lungs compared to normoxic (Nx) rat lungs and SuHx rat lungs treated with Ab93. Isotype: 10 mg/kg, IP. Ab93: 10 mg/kg, IP. Data analysis was performed using GraphPad Prism 9 (GraphPad Software). Results are shown as means with SD. Statistical significance was analyzed by t test (nonparametric tests). Values of P<0.05 were considered significant. N=8 for Nx, N=6 for SuHx_Iso and SuHx_Ab93.

FIGS. 15A-15D depict concentration of CXCL12 (FIG. 15A), CCL2 (FIG. 15B), PDGFBB (FIG. 15C), and Endothelin) (FIG. 15D) proteins in HPMVEC culture medium in response to BMP9 treatment. Data analysis was performed using GraphPad Prism 9 (GraphPad Software). Results are shown as means with SD. Statistical significance was analyzed by t test (nonparametric tests). *P<0.05.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:
E1. An isolated antibody that binds to bone morphogenetic protein-9 (BMP9) and comprises a heavy chain variable region (VH) and a light chain variable region (VL), wherein:
   the VH complementarity determining region one (CDR1) comprises an amino acid sequence selected from the group consisting of SEQ ID NO: 9, SEQ ID NO: 24, SEQ ID NO: 36, SEQ ID NO: 45, and SEQ ID NO: 53,
   the VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76,
   the VH CDR3 comprises an amino acid sequence selected from the group consisting of SEQ ID NO: 11, SEQ ID NO: 26, SEQ ID NO: 38, SEQ ID NO: 47, and SEQ ID NO: 62,
   the VL CDR1 comprises an amino acid sequence selected from the group consisting of SEQ ID NO: 2, SEQ ID NO: 19, SEQ ID NO: 30, SEQ ID NO: 50, SEQ ID NO: 57, SEQ ID NO: 65, and SEQ ID NO: 73,
   the VL CDR2 comprises an amino acid sequence selected from the group consisting of SEQ ID NO: 3, SEQ ID NO: 20, SEQ ID NO: 31, SEQ ID NO: 41, and SEQ ID NO: 66, and
   the VL CDR3 comprises an amino acid sequence selected from the group consisting of SEQ ID NO: 4, SEQ ID NO: 21, SEQ ID NO: 32, SEQ ID NO: 42, SEQ ID NO: 58, and SEQ ID NO: 67.
E2. An isolated antibody that binds to bone morphogenetic protein-9 (BMP9) and comprises a heavy chain variable region (VH) and a light chain variable region (VL), wherein:
   (i) the VH CDR1 comprises the amino acid sequence of SEQ ID NO: 80, the VH CDR2 comprises the amino acid sequence of SEQ ID NO: 81, and the VH CDR3 comprises the amino acid sequence of SEQ ID NO: 26, and the VL CDR1 comprises the amino acid sequence of SEQ ID NO: 82, the VL CDR2 comprises the amino acid sequence of SEQ ID NO: 83, and the VL CDR3 comprises the amino acid sequence of SEQ ID NO: 84;
   (ii) the VH CDR1 comprises the amino acid sequence of SEQ ID NO: 9, the VH CDR2 comprises the amino acid sequence of SEQ ID NO: 10, and the VH CDR3 comprises the amino acid sequence of SEQ ID NO: 11, and the VL CDR1 comprises the amino acid sequence of SEQ ID NO: 2, the VL CDR2 comprises the amino acid sequence of SEQ ID NO: 3, and the VL CDR3 comprises the amino acid sequence of SEQ ID NO: 4;
   (iii) the VH CDR1 comprises the amino acid sequence of SEQ ID NO: 24, the VH CDR2 comprises the amino acid sequence of SEQ ID NO: 25, and the VH CDR3 comprises the amino acid sequence of SEQ ID NO: 26, and the VL CDR1 comprises the amino acid sequence of SEQ ID NO: 19, the VL CDR2 comprises the amino acid sequence of SEQ ID NO: 20, and the VL CDR3 comprises the amino acid sequence of SEQ ID NO: 21;
   (iv) the VH CDR1 comprises the amino acid sequence of SEQ ID NO: 36, the VH CDR2 comprises the amino acid sequence of SEQ ID NO: 37, and the VH CDR3 comprises the amino acid sequence of SEQ ID NO: 38, and the VL CDR1 comprises the amino acid sequence of SEQ ID NO: 30, the VL CDR2 comprises the amino acid sequence of SEQ ID NO: 31, and the VL CDR3 comprises the amino acid sequence of SEQ ID NO: 32;
   (v) the VH CDR1 comprises the amino acid sequence of SEQ ID NO: 45, the VH CDR2 comprises the amino acid sequence of SEQ ID NO: 46, and the VH CDR3 comprises the amino acid sequence of SEQ ID NO: 47, and the VL CDR1 comprises the amino acid sequence of SEQ ID NO: 2, the VL CDR2 comprises the amino acid sequence of SEQ ID NO: 41, and the VL CDR3 comprises the amino acid sequence of SEQ ID NO: 42;
   (vi) the VH CDR1 comprises the amino acid sequence of SEQ ID NO: 53, the VH CDR2 comprises the amino acid sequence of SEQ ID NO: 54, and the VH CDR3 comprises the amino acid sequence of SEQ ID NO: 26, and the VL CDR1 comprises the amino acid sequence of SEQ ID NO: 50, the VL CDR2 comprises the amino acid sequence of SEQ ID NO: 20, and the VL CDR3 comprises the amino acid sequence of SEQ ID NO: 21;
   (vii) the VH CDR1 comprises the amino acid sequence of SEQ ID NO: 45, the VH CDR2 comprises the amino acid sequence of SEQ ID NO: 61, and the VH CDR3 comprises the amino acid sequence of SEQ ID NO: 62, and the VL CDR1 comprises the amino acid sequence of SEQ ID NO: 57, the VL CDR2 comprises the amino acid sequence of SEQ ID NO: 41, and the VL CDR3 comprises the amino acid sequence of SEQ ID NO: 58;
   (viii) the VH CDR1 comprises the amino acid sequence of SEQ ID NO: 53, the VH CDR2 comprises the amino acid sequence of SEQ ID NO: 70, and the VH CDR3 comprises the amino acid sequence of SEQ ID NO: 26, and the VL CDR1 comprises the amino acid sequence of SEQ ID NO: 65, the VL CDR2 comprises the amino acid sequence of SEQ ID NO: 66, and the VL CDR3 comprises the amino acid sequence of SEQ ID NO: 67; or
   (ix) the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.
E3. The isolated antibody of E2, wherein:
   (i) the VH CDR1 comprises the amino acid sequence of SEQ ID NO: 80, the VH CDR2 comprises the amino acid sequence of SEQ ID NO: 81, and the VH CDR3 comprises the amino acid sequence of SEQ ID NO: 26, and the VL CDR1 comprises the amino acid sequence of SEQ ID NO: 82, the VL CDR2 comprises the amino acid sequence of SEQ ID NO: 83, and the VL CDR3 comprises the amino acid sequence of SEQ ID NO: 84;
   (ii) the VH CDR1 comprises the amino acid sequence of SEQ ID NO: 24, the VH CDR2 comprises the amino acid sequence of SEQ ID NO: 25, and the VH CDR3 comprises the amino acid sequence of SEQ ID NO: 26, and the VL CDR1 comprises the amino acid sequence of SEQ ID NO: 19, the VL CDR2 comprises the amino acid sequence of SEQ ID NO: 20, and the VL CDR3 comprises the amino acid sequence of SEQ ID NO: 21;
   (iii) the VH CDR1 comprises the amino acid sequence of SEQ ID NO: 53, the VH CDR2 comprises the amino acid sequence of SEQ ID NO: 54, and the VH CDR3 comprises the amino acid sequence of SEQ ID NO: 26, and the VL CDR1 comprises the amino acid sequence of SEQ ID NO: 50, the VL CDR2 comprises the amino acid sequence of SEQ ID NO: 20, and the VL CDR3 comprises the amino acid sequence of SEQ ID NO: 21;
   (iv) the VH CDR1 comprises the amino acid sequence of SEQ ID NO: 53, the VH CDR2 comprises the amino acid sequence of SEQ ID NO: 70, and the VH CDR3 comprises the amino acid sequence of SEQ ID NO: 26, and the VL CDR1 comprises the amino acid sequence of SEQ ID NO: 65, the VL CDR2 comprises the amino acid sequence of SEQ ID NO: 66, and the VL CDR3 comprises the amino acid sequence of SEQ ID NO: 67; or
   (v) the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.
E4. An isolated antibody that binds to bone morphogenetic protein-9 (BMP9) and comprises a heavy chain variable region (VH) and a light chain variable region (VL), wherein the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.
E5. The isolated antibody of any one of E1-E4, wherein the VH comprises the amino acid sequence of SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, or a variant thereof comprising one to four amino acid substitutions at residues that are not within a CDR, and the VL comprises the amino acid sequence of SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74, or a variant thereof comprising one to four amino acid substitutions at residues that are not within a CDR.
E6. The isolated antibody of any one of E1-E4, wherein the VH comprises an amino acid sequence at least 90%, at least 91%, at least 92%, at least 93%, at least 94%, at least 95%, at least 96%, at least 97%, at least 98%, at least 99%, or 100% identical to SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, and the VL comprises an amino acid sequence at least 90%, at least 91%, at least 92%, at least 93%, at least 94%, at least 95%, at least 96%, at least 97%, at least 98%, at least 99%, or 100% identical to SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74.
E7. The isolated antibody of any one of E5-E6, wherein the antibody comprises:
   (i) a VH comprising the amino acid sequence of SEQ ID NO: 16 and a VL comprising the amino acid sequence of SEQ ID NO: 6;
   (ii) a VH comprising the amino acid sequence of SEQ ID NO: 27 and a VL comprising the amino acid sequence of SEQ ID NO: 22;
   (iii) a VH comprising the amino acid sequence of SEQ ID NO: 39 and a VL comprising the amino acid sequence of SEQ ID NO: 33;
   (iv) a VH comprising the amino acid sequence of SEQ ID NO: 48 and a VL comprising the amino acid sequence of SEQ ID NO: 43;
   (v) a VH comprising the amino acid sequence of SEQ ID NO: 55 and a VL comprising the amino acid sequence of SEQ ID NO: 51;
   (vi) a VH comprising the amino acid sequence of SEQ ID NO: 63 and a VL comprising the amino acid sequence of SEQ ID NO: 59;
   (vii) a VH comprising the amino acid sequence of SEQ ID NO: 71 and a VL comprising the amino acid sequence of SEQ ID NO: 68; or
   (viii) a VH comprising the amino acid sequence of SEQ ID NO: 77 and a VL comprising the amino acid sequence of SEQ ID NO: 74.
E8. The isolated antibody of E7, wherein the antibody comprises:
   (i) a VH comprising the amino acid sequence of SEQ ID NO: 27 and a VL comprising the amino acid sequence of SEQ ID NO: 22;
   (ii) a VH comprising the amino acid sequence of SEQ ID NO: 55 and a VL comprising the amino acid sequence of SEQ ID NO: 51;
   (iii) a VH comprising the amino acid sequence of SEQ ID NO: 71 and a VL comprising the amino acid sequence of SEQ ID NO: 68;
   (iv) a VH comprising the amino acid sequence of SEQ ID NO: 77 and a VL comprising the amino acid sequence of SEQ ID NO: 74;
   (v) a VH comprising an amino acid sequence at least 90%, at least 91%, at least 92%, at least 93%, at least 94%, at least 95%, at least 96%, at least 97%, at least 98%, at least 99%, or 100% identical to SEQ ID NO: 27 and a VL comprising an amino acid sequence at least 90%, at least 91%, at least 92%, at least 93%, at least 94%, at least 95%, at least 96%, at least 97%, at least 98%, at least 99%, or 100% identical to SEQ ID NO: 22;
   (vi) a VH comprising an amino acid sequence at least 90%, at least 91%, at least 92%, at least 93%, at least 94%, at least 95%, at least 96%, at least 97%, at least 98%, at least 99%, or 100% identical to SEQ ID NO: 55 and a VL comprising an amino acid sequence at least 90%, at least 91%, at least 92%, at least 93%, at least 94%, at least 95%, at least 96%, at least 97%, at least 98%, at least 99%, or 100% identical to SEQ ID NO: 51;
   (vii) a VH comprising an amino acid sequence at least 90%, at least 91%, at least 92%, at least 93%, at least 94%, at least 95%, at least 96%, at least 97%, at least 98%, at least 99%, or 100% identical to SEQ ID NO: 71 and a VL comprising an amino acid sequence at least 90%, at least 91%, at least 92%, at least 93%, at least 94%, at least 95%, at least 96%, at least 97%, at least 98%, at least 99%, or 100% identical to SEQ ID NO: 68; or
   (viii) a VH comprising an amino acid sequence at least 90%, at least 91%, at least 92%, at least 93%, at least 94%, at least 95%, at least 96%, at least 97%, at least 98%, at least 99%, or 100% identical to SEQ ID NO: 77 and a VL comprising an amino acid sequence at least 90%, at least 91%, at least 92%, at least 93%, at least 94%, at least 95%, at least 96%, at least 97%, at least 98%, at least 99%, or 100% identical to SEQ ID NO: 74.
E9. The isolated antibody of E8, wherein the antibody comprises a VH comprising the amino acid sequence of SEQ ID NO: 77 and a VL comprising the amino acid sequence of SEQ ID NO: 74.
E10. A portion of an isolated antibody, wherein the isolated antibody binds to BMP9 and comprises a heavy chain variable region (VH) and a light chain variable region (VL), wherein:
   the VH CDR1 comprises an amino acid sequence selected from the group consisting of SEQ ID NO: 9, SEQ ID NO: 24, SEQ ID NO: 36, SEQ ID NO: 45, and SEQ ID NO: 53:
   the VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76;
   the VH CDR3 comprises an amino acid sequence selected from the group consisting of SEQ ID NO: 11, SEQ ID NO: 26, SEQ ID NO: 38, SEQ ID NO: 47, and SEQ ID NO: 62;
   the VL CDR1 comprises an amino acid sequence selected from the group consisting of SEQ ID NO: 2, SEQ ID NO: 19, SEQ ID NO: 30, SEQ ID NO: 50, SEQ ID NO: 57, SEQ ID NO: 65, and SEQ ID NO: 73;
   the VL CDR2 comprises an amino acid sequence selected from the group consisting of SEQ ID NO: 3, SEQ ID NO: 20, SEQ ID NO: 31, SEQ ID NO: 41, and SEQ ID NO: 66; and
   the VL CDR3 comprises an amino acid sequence selected from the group consisting of SEQ ID NO: 4, SEQ ID NO: 21, SEQ ID NO: 32, SEQ ID NO: 42, SEQ ID NO: 58, and SEQ ID NO: 67, and
   wherein the portion binds to BMP9.
E11. The portion of E10, wherein the portion comprises:
   a VH CDR1 comprising an amino acid sequence selected from the group consisting of SEQ ID NO: 9, SEQ ID NO: 24, SEQ ID NO: 36, SEQ ID NO: 45, and SEQ ID NO: 53;
   a VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76; and
   a VH CDR3 comprises an amino acid sequence selected from the group consisting of SEQ ID NO: 11, SEQ ID NO: 26, SEQ ID NO: 38, SEQ ID NO: 47, and SEQ ID NO: 62.
E12. An isolated antibody that binds to BMP9 and comprises a VH encoded by the nucleic acid sequence of the insert of the plasmid deposited with the ATCC and having the Accession Number PTA-127292, a VL encoded by the nucleic acid sequence of the insert of the plasmid deposited with the ATCC and having the Accession Number PTA-127293, or both.
E13. An isolated antibody comprising:
   (i) the VH CDR1, VH CDR2, and VH CDR3 sequences of SEQ ID NO: 16 and the VL CDR1, VL CDR2, and VL CDR3 sequences of SEQ ID NO: 6;
   (ii) the VH CDR1, VH CDR2, and VH CDR3 sequences of SEQ ID NO: 27 and the VL CDR1, VL CDR2, and VL CDR3 sequences of SEQ ID NO: 22;
   (iii) the VH CDR1, VH CDR2, and VH CDR3 sequences of SEQ ID NO: 39 and the VL CDR1, VL CDR2, and VL CDR3 sequences of SEQ ID NO: 33;
   (iv) the VH CDR1, VH CDR2, and VH CDR3 sequences of SEQ ID NO: 48 and the VL CDR1, VL CDR2, and VL CDR3 sequences of SEQ ID NO: 43;
   (v) the VH CDR1, VH CDR2, and VH CDR3 sequences of SEQ ID NO: 55 and the VL CDR1, VL CDR2, and VL CDR3 sequences of SEQ ID NO: 51;
   (vi) the VH CDR1, VH CDR2, and VH CDR3 sequences of SEQ ID NO: 63 and the VL CDR1, VL CDR2, and VL CDR3 sequences of SEQ ID NO: 59;
   (vii) the VH CDR1, VH CDR2, and VH CDR3 sequences of SEQ ID NO: 71 and the VL CDR1, VL CDR2, and VL CDR3 sequences of SEQ ID NO: 68; or
   (viii) the VH CDR1, VH CDR2, and VH CDR3 sequences of SEQ ID NO: 77 and the VL CDR1, VL CDR2, and VL CDR3 sequences of SEQ ID NO: 74.
E14. The isolated antibody of any one of E1-E13, wherein the antibody comprises a human V_{κ} or V_{λ} light chain constant domain.
E15. The isolated antibody of E14, wherein the antibody comprises a human V_{λ} light chain constant domain.
E16. The isolated antibody of any one of E1-E15, wherein the antibody comprises a heavy chain constant domain.
E17. The isolated antibody of E16, wherein the heavy chain constant domain comprises an IgA (for example IgA₁ or IgA₂), IgD, IgE, IgM, or IgG (for example IgG₁, IgG₂, IgG₃, or IgG₄).
E18. The isolated antibody of E17, wherein the heavy chain constant domain comprises an IgG.
E19. The isolated antibody of E18, wherein the IgG is selected from the group consisting of IgG₁, IgG₂, IgG₃, and IgG₄.
E20. The isolated antibody of E19, wherein the IgG is IgG₁.
E21. The isolated antibody of any one of E1-E20 wherein the antibody comprises an Fc domain.
E22. The isolated antibody of E21, wherein the Fc domain comprises an IgG₁ heavy chain CH2 domain and an IgG₁ heavy chain CH3 domain.
E23. An isolated antibody comprising:
   (i) a heavy chain comprising an amino acid sequence selected from the group consisting of SEQ ID NO: 8, SEQ ID NO: 23, SEQ ID NO: 35, SEQ ID NO: 44, SEQ ID NO: 52, SEQ ID NO: 60, SEQ ID NO: 69, and SEQ ID NO: 75; and
   (ii) a light chain comprising an amino acid sequence selected from the group consisting of SEQ ID NO: 1, SEQ ID NO: 18, SEQ ID No: 29, SEQ ID NO: 40, SEQ ID NO: 49, SEQ ID NO: 56, SEQ ID NO: 64, and SEQ ID NO: 72.
E24. An isolated antibody comprising:
   (i) a heavy chain comprising an amino acid sequence at least 90%, at least 91%, at least 92%, at least 93%, at least 94%, at least 95%, at least 96%, at least 97%, at least 98%, at least 99%, or 100% identical to SEQ ID NO: 8, SEQ ID NO: 23, SEQ ID NO: 35, SEQ ID NO: 44, SEQ ID NO: 52, SEQ ID NO: 60, SEQ ID NO: 69, or SEQ ID NO: 75; and
   (ii) a light chain comprising an amino acid sequence at least 90%, at least 91%, at least 92%, at least 93%, at least 94%, at least 95%, at least 96%, at least 97%, at least 98%, at least 99%, or 100% identical to SEQ ID NO: 1, SEQ ID NO: 18, SEQ ID No: 29, SEQ ID NO: 40, SEQ ID NO: 49, SEQ ID NO: 56, SEQ ID NO: 64, or SEQ ID NO: 72.
E25. An isolated antibody comprising:
   (i) a HC comprising, or consisting of, the amino acid sequence of SEQ ID NO: 8 and a LC comprising, or consisting of, the amino acid sequence of SEQ ID NO: 1;
   (ii) a HC comprising, or consisting of, the amino acid sequence of SEQ ID NO: 23 and a LC comprising, or consisting of, the amino acid sequence of SEQ ID NO: 18;
   (iii) a HC comprising, or consisting of, the amino acid sequence of SEQ ID NO: 35 and a LC comprising, or consisting of, the amino acid sequence of SEQ ID NO: 29;
   (iv) a HC comprising, or consisting of, the amino acid sequence of SEQ ID NO: 44 and a LC comprising, or consisting of, the amino acid sequence of SEQ ID NO: 40;
   (v) a HC comprising, or consisting of, the amino acid sequence of SEQ ID NO: 52 and a LC comprising, or consisting of, the amino acid sequence of SEQ ID NO: 49;
   (vi) a HC comprising, or consisting of, the amino acid sequence of SEQ ID NO: 60 and a LC comprising, or consisting of, the amino acid sequence of SEQ ID NO: 56;
   (vii) a HC comprising, or consisting of, the amino acid sequence of SEQ ID NO: 69 and a LC comprising, or consisting of, the amino acid sequence of SEQ ID NO: 64; or
   (viii) a HC comprising, or consisting of, the amino acid sequence of SEQ ID NO: 75 and a LC comprising, or consisting of, the amino acid sequence of SEQ ID NO: 72.
E26. An isolated antibody comprising:
   (i) a HC comprising, or consisting of, the amino acid sequence of SEQ ID NO: 23 and a LC comprising, or consisting of, the amino acid sequence of SEQ ID NO: 18;
   (ii) a HC comprising, or consisting of, the amino acid sequence of SEQ ID NO: 52 and a LC comprising, or consisting of, the amino acid sequence of SEQ ID NO: 49;
   (iii) a HC comprising, or consisting of, the amino acid sequence of SEQ ID NO: 69 and a LC comprising, or consisting of, the amino acid sequence of SEQ ID NO: 64;
   (iv) a HC comprising, or consisting of, the amino acid sequence of SEQ ID NO: 75 and a LC comprising, or consisting of, the amino acid sequence of SEQ ID NO: 72;
   (v) a HC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 23 and a LC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 18;
   (vi) a HC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 52 and a LC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 49;
   (vii) a HC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 69 and a LC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 64; or
   (viii) a HC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 75 and a LC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 72.
E27. An isolated antibody comprising:
   (i) a HC comprising, or consisting of, the amino acid sequence of SEQ ID NO: 75 and a LC comprising, or consisting of, the amino acid sequence of SEQ ID NO: 72; or
   (ii) a HC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 75 and a LC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 72.
E28. An isolated antibody that binds to BMP9, wherein the antibody binds to an epitope on BMP9 comprising one or more amino acid residues selected from the group consisting of Ala28, Pro29, Ser83, Leu85, Met90, Val92, Pro93, Leu95, and Tyr97 of SEQ ID NO: 79.
E29. The isolated antibody of E28, wherein the antibody binds Ala28, Pro29, Ser83, Leu85, Met90, Val92, Pro93, Leu95, and Tyr97 of SEQ ID NO: 79.
E30. The isolated antibody of any one of E28-E29, wherein the antibody further binds one or more amino acid residues selected from the group consisting of Pro81, Tyr86, Asp88, Asp89, Thr94, Lys96, His98, Tyr99, and Glu100 of SEQ ID NO: 79.
E31. The isolated antibody of E30, wherein the antibody binds Pro81, Tyr86, Asp88, Asp89, Thr94, Lys96, His98, Tyr99, and Glu100 of SEQ ID NO: 79.
E32. The isolated antibody of any one of E28-E31, wherein the antibody further binds one or more amino acid residues selected from the group consisting of Ile27, Lys30, and Glu31 of SEQ ID NO: 79.
E33. The isolated antibody of E32, wherein the antibody binds Ile27, Lys30, and Glu31 of SEQ ID NO: 79.
E34. An isolated antibody that binds to BMP9, wherein the antibody binds to an epitope on BMP9 comprising one or more amino acid residues selected from the group consisting of Ile27, Ala28, Pro29, Lys30, Glu31, Tyr32, Ser80, Pro81, Ile82, Ser83, Val84, Leu85, Tyr86, Asp88, Asp89, Met90, Val92, Pro93, Thr94, Leu95, Lys96, Tyr97, His98, Tyr99, and Glu100 of SEQ ID NO: 79.
E35. An isolated antibody that binds to BMP9, wherein the antibody binds to an epitope on BMP9 comprising Ile27, Ala28, Pro29, Lys30, Glu31, Tyr32, Ser80, Pro81, Ile82, Ser83, Val84, Leu85, Tyr86, Asp88, Asp89, Met90, Val92, Pro93, Thr94, Leu95, Lys96, Tyr97, His98, Tyr99, and Glu100 of SEQ ID NO: 79.
E36. An isolated antibody that binds to BMP9, wherein the antibody binds to an epitope on BMP9 comprising one or more amino acid residues selected from the group consisting of Ala28, Pro29, Pro81, Ser83, Leu85, Tyr86, Asp88, Asp89, Met90, Val92, Pro93, Thr94, Leu95, Lys96, Tyr97, His98, Tyr99, and Glu100 of SEQ ID NO: 79.
E37. An isolated antibody that binds to BMP9, wherein the antibody binds to an epitope on BMP9 comprising Ala28, Pro29, Pro81, Ser83, Leu85, Tyr86, Asp88, Asp89, Met90, Val92, Pro93, Thr94, Leu95, Lys96, Tyr97, His98, Tyr99, and Glu100 of SEQ ID NO: 79.
E38. An isolated antibody that binds to BMP9, wherein the antibody binds to an epitope on BMP9 comprising one or more amino acid residues selected from the group consisting of Arg14, Ile27, Ala28, Pro29, Lys30, Glu31, Ser83, Leu85, Met90, Val92, Pro93, Leu95, and Tyr97 of SEQ ID NO: 79.
E39. An isolated antibody that binds to BMP9, wherein the antibody binds to an epitope on BMP9 comprising Arg14, Ile27, Ala28, Pro29, Lys30, Glu31, Ser83, Leu85, Met90, Val92, Pro93, Leu95, and Tyr97 of SEQ ID NO: 79.
E40. The isolated antibody of any one of E28-E39, wherein the epitope on BMP9 does not comprise amino acid resides 21-25, 43-60 of SEQ ID NO: 79.
E41. The isolated antibody of any one of E1-E40, wherein the antibody binds human BMP9 with an affinity of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM at 37° C. as measured by surface plasmon resonance. E42. The isolated antibody of E41, wherein the antibody binds human BMP9 with an affinity of about 30.48 pM or about 87.41 pM at 37° C. as measured by surface plasmon resonance.
E43. The isolated antibody of any one of E1-E42, wherein the antibody binds cyno BMP9 with an affinity of about 10 pM, about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM at 37° C. as measured by surface plasmon resonance.
E44. The isolated antibody of E43, wherein the antibody binds cyno BMP9 with an affinity of about 20.23 pM or about 65.82 pM at 37° C. as measured by surface plasmon resonance. E45. The isolated antibody of any one of E1-E44, wherein the antibody binds rat BMP9 with an affinity of about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM at 37° C. as measured by surface plasmon resonance.
E46. The isolated antibody of E45, wherein the antibody binds rat BMP9 with an affinity of about 41.75 pM or about 98.63 pM at 37° C. as measured by surface plasmon resonance.
E47. The antibody of any one of E28-E31, E34-E37, E40-E46, wherein the antibody comprises:
   (i) a heavy chain variable region (VH) CDR3 of SEQ ID NO: 26 and a light chain variable region (VL) CDR3 of SEQ ID NO: 84;
   (ii) a heavy chain variable region (VH) CDR3 of SEQ ID NO: 26 and a light chain variable region (VL) CDR3 of SEQ ID NO: 21; or
   (iii) a heavy chain variable region (VH) CDR3 of SEQ ID NO: 26 and a light chain variable region (VL) CDR3 of SEQ ID NO: 67.
E48. The isolated antibody of any one of E1-E47, wherein the antibody:
   (i) binds to human BMP9 and inhibits binding of human BMP9 to a BMP9 type II receptor (e.g., BMPRII, ActRIIA or ActRIIB); or
   (ii) binds to human BMP9 and inhibits binding of human BMP9 to a BMP9 type II receptor (e.g., BMPRII, ActRIIA or ActRIIB) and weakly inhibits binding of human BMP9 to a BMP9 type 1 receptor (e.g., ALK1).
E49. The isolated antibody of any one of E1-48, wherein the antibody binds to human BMP9 and inhibits binding of human BMP9 to human endothelial cells.
E50. The isolated antibody of any one of E1-E49, wherein the antibody binds human BMP9 but does not bind other human transforming growth factor beta (TGFβ) superfamily ligands (e.g., GDF8, GDF11, Activin A, GDF9, TGFβ1 and BMP10).
E51. The isolated antibody of any one of E1-E50, wherein the antibody inhibits BMP9 activity measured by phospho-SMAD1/5/9 nuclear translocation in endothelial cells.
E52. The isolated antibody of E51, wherein the antibody inhibits human BMP9 activity with an IC₅₀ of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, about 145 pM, about 150 pM, about 155 pM, about 160 pM, about 170 pM, about 180 pM, about 190 pM, about 200 pM, about 225 pM, about 250 pM, about 300 pM, about 350 pM, about 400 pM, about 450 pM, about 500 pM, about 600 pM, about 700 pM, about 800 pM, about 900 pM, about 1000 pM, about 2 nM, about 10 nM, about 20 nM, about 30 nM, about 40 nM, about 50 nM, about 60 nM, about 70 nM, about 80 nM, about 90 nM or about 100 nM.
E53. The isolated antibody of E51, wherein the antibody inhibits rat BMP9 activity with an IC₅₀ of about 110 pM, about 115 pM, about 120 pM, about 125 pM, about 130 pM, about 135 pM, about 140 pM, about 150 pM, about 175 pM, about 200 pM, about 225 pM, about 250 pM, about 275 pM, or about 300 pM.
E54. The isolated antibody of E51, wherein the antibody inhibits cyno BMP9 activity with an IC₅₀ of about 17 pM, about 20 pM, about 30 pM, about 40 pM, about 50 pM, about 60 pM, about 70 pM, about 80 pM, about 90 pM about 100 pM, about 110 pM, about 115 pM, about 120 pM, about 125 pM, about 130 pM, about 135 pM, or about 140 pM.
E55. The isolated antibody of any one of E1-E54, wherein the antibody inhibits BMP9 activity measured by phospho-SMAD2 nuclear translocation in endothelial cells.
E56. The isolated antibody of E55, wherein the antibody inhibits BMP9 activity with an IC₅₀ of about 10 pM, about 15 pM, about 20 pM, about 25 pM, about 30 pM, about 35 pM, about 40 pM, about 50 pM, about 100 pM, about 250 pM, about 500 pM, about 750 pM, about 1000 pM, about 10 nM, about 100 nM, about 200 nM, about 300 nM or about 350 nM.
E57. The isolated antibody of any one of E1-E56, wherein the antibody reduces right ventricular systolic pressure in a subject by at least 5%, at least 10%, at least 15%, at least 20%, at least 25%, at least 30%, at least 35%, at least 40%, at least 45%, at least 50%, at least 55%, at least 60%, at least 65%, or at least 70% as compared to before antibody administration.
E58. The isolated antibody of any one of E1-E57, wherein the antibody reduces right ventricular hypertrophy in a subject by at least 2%, at least 5%, at least 10%, at least 15%, at least 20%, at least 25%, at least 30%, at least 35%, at least 40%, at least 45%, at least 50%, at least 55%, at least 60%, at least 65%, or at least 70% as compared to before antibody administration.
E59. The isolated antibody of any one of E57-E58, wherein the subject is a Sugen-Hypoxia animal model of pulmonary arterial hypertension (PAH).
E60. An isolated polynucleotide encoding the antibody of any one E1-E59, E91, or E92.
E61. The polynucleotide of E60, wherein said polynucleotide is RNA.
E62. The polynucleotide of E61, wherein said polynucleotide comprises at least one chemical modification.
E63. The polynucleotide of E62, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine.
E64. The polynucleotide of E61, wherein said polynucleotide does not comprise a chemical modification.
E65. An isolated nucleic acid encoding the VH, VL, or both, of an antibody that binds BMP9, wherein the nucleic acid comprises the nucleic acid sequence of SEQ ID NO: 85, the nucleic acid sequence of SEQ ID NO: 86, or both.
E66. An isolated nucleic acid encoding the heavy chain, light chain, or both, of an antibody that binds BMP9, wherein said nucleic acid comprises the nucleic acid sequence of SEQ ID NO: 87, the nucleic acid sequence of SEQ ID NO: 88, or both.
E67. An isolated nucleic acid encoding the VH, VL, or both, of an antibody that binds to BMP9, wherein said nucleic acid comprises the nucleic acid sequence of the insert of the plasmid deposited with the ATCC and having the Accession Number PTA-127292, the nucleic acid sequence of the insert of the plasmid deposited with the ATCC and having the Accession Number PTA-127293, or both.
E68. A vector comprising the polynucleotide of any one of E60-E64 or the nucleic acid of any one of E65-E67.
E69. An isolated host cell comprising the polynucleotide of any one of E60-E64 or the nucleic acid of any one of E65-E67.
E70. The host cell of E69, wherein the cell is a mammalian cell or an insect cell.
E71. The host cell of E70, wherein the host cell is a mammalian cell selected from the group consisting of a CHO cell, an HEK-293 cell, an NS0 cell, a PER.C6® cell, or a Sp2.0 cell.
E72. A method of producing an antibody, comprising:
   culturing the host cell of any one of E69-E71 under conditions that result in production of the antibody; and recovering the antibody.
E73. A pharmaceutical composition comprising a therapeutically effective amount of the antibody of any one of E1-E59, E90, or E91 and a pharmaceutically acceptable carrier.
E74. The pharmaceutical composition of E73, wherein the composition comprises 1.12 mg/mL L-histidine, 2.67 mg/mL L-histidine hydrochloride monohydrate, 85 mg/mL sucrose, 0.05 mg/mL EDTA disodium dihydrate, and 0.2 mg/mL polysorbate 80 at pH 5.8.
E75. The pharmaceutical composition of any one of E73-E74, wherein the composition comprises 20 mM histidine, 8.5% sucrose, and 0.02% polysorbate 80, 0.005% EDTA at pH 5.8.
E76. The pharmaceutical composition of any one of E72-E74, wherein the composition comprises about 2 mg/mL, 5 mg/mL, 10 mg/mL, 15 mg/mL, 20 mg/mL, 25 mg/mL, 50 mg/mL, 75 mg/mL, 100 mg/mL, 125 mg/mL, 150 mg/mL, 175 mg/mL, or 200 mg/mL antibody.
E77. The pharmaceutical composition of any one of E73-E76 wherein the composition comprises about 50 mg/mL, about 100 mg/mL, about 150 mg/ml, or about 200 mg/mL antibody.
E78. The pharmaceutical composition of any one of E73-E77, wherein the dose is a 1 mL dose.
E79. The pharmaceutical composition of E73-E78, wherein the composition is suitable for subcutaneous (SC) and/or intravenous (IV) administration.
E80. A method for reducing the expression or activity of BMP9 in a subject, the method comprising administering to a subject in need thereof a therapeutically effective amount of the pharmaceutical composition of any one of E73-E79 or the antibody of any one of E1-E59, E90, or E91.
E81. A method of treating hypertension in a subject, the method comprising administering to a subject in need thereof a therapeutically effective amount of the pharmaceutical composition of any one of claims E73-E79 or the antibody of E1-E59, E90, or E91.
E82. The method of E81, wherein the hypertension is pulmonary hypertension.
E83. A method of treating pulmonary arterial hypertension (PAH) in a subject, the method comprising administering to a subject in need thereof a therapeutically effective amount of the pharmaceutical composition of any one of E73-E79 or the antibody of E1-E59, E90, or E91.
E84. The method of any one of E80-E83, wherein the antibody is administered in combination with a therapeutically effective amount of one or more additional therapeutically active compounds or treatment modalities effective in treating at least one sign and/or symptom of hypertension, optionally, pulmonary arterial hypertension.
E85. The method of E84, wherein the combination therapies are administered according to the same dosing regimen (e.g., both therapies are administered daily) or according to different dosing regimens (e.g., one therapy is administered daily, the other therapy is administered weekly).
E86. An isolated antibody of any one of E1-E59, E90, or E91, or the pharmaceutical composition of any one of E73-E79, for use as a medicament.
E87. An isolated antibody of any one of E1-E59, E90, or E91, or the pharmaceutical composition of any one of E73-E79, for use in the treatment of at least one sign and/or symptom of hypertension, optionally pulmonary arterial hypertension.
E88. Use of the isolated antibody of any one of E1-E59, E90, or E91, or the pharmaceutical 25 composition of any one of E73-E79, in the manufacture of a medicament for treating at least one sign and/or symptom of hypertension, optionally, pulmonary arterial hypertension.
E89. The pharmaceutical composition of any one of E73-E79, or the isolated antibody of any one of E1-E59, E90, or E91, for the treatment of at least one sign and/or symptom of 30 hypertension, optionally, pulmonary arterial hypertension.
E90. Use of the isolated antibody of any one of E1-E59, E91, or E92, or the pharmaceutical composition of any one of E73-E79, to treat at least one sign and/or symptom of hypertension, optionally, pulmonary arterial hypertension.
E91. The isolated antibody of any one of E1-E59, wherein the isolated antibody inhibits one or more proteins correlated with hypertension, optionally, pulmonary hypertension.
E92. The isolated antibody of any one of E1-E59 or E91, wherein the isolated antibody inhibits CXCL12, IGFBP4, INHBA, MALL, FRZB, CPE, CCL2, PDGFBB, Endothelin-1, or a combination thereof.
E93. The method of any one of E80-E85, wherein of the pharmaceutical composition of any one of E73-E79 or the antibody of any one of E1-E59, E90, or E91 inhibits one or more proteins correlated with hypertension, optionally, pulmonary hypertension.
E94. The method of any one of E80-E85 or E93, wherein of the pharmaceutical composition of any one of E73-E79 or the antibody of any one of E1-E59, E91, or E92 inhibits CXCL12, IGFBP4, INHBA, MALL, FRZB, CPE, CCL2, PDGFBB, Endothelin-1, or a combination thereof.
E100. A method for monitoring the effectiveness of a pulmonary arterial hypertension (PAH) therapy in a subject in need thereof comprising: (i) administering a PAH therapy to the subject; (ii) determining the level of one or more biomarkers in a biological sample obtained from the subject; and (iii) comparing the level of the one or more biomarkers from step (ii) to the level of the same one or more biomarkers before administration of the PAH therapy, wherein a change (e.g., a decrease) in the level of the one or more biomarkers after administration of the PAH therapy indicates that the therapy is effective, and wherein the PAH therapy comprises the isolated antibody of any one of E1-E59, E91, or E92.
E101. The method of E100, wherein the biological sample comprises urine, blood, serum, plasma, saliva, sputum, exhaled breath condensate, bronchoalveolar lavage fluid, cerebrospinal fluid, tears, tissue, or mucus, or a combination thereof.
E102. The method of any one of E100-E101, wherein determining the level of one or more biomarkers comprises determining mRNA levels.
E103. The method of any one of E100-E101, wherein determining the level of one or more biomarkers comprises determining protein levels.

The section headings used herein are for organizational purposes only and are not to be construed as limiting the subject matter described.

All references cited herein, including literature references, patent applications, patent publications, UniProtKB accession numbers, and GenBank accession numbers, are hereby specifically and expressly incorporated by reference, as if each individual reference were specifically and individually indicated to be incorporated by reference in its entirety. When definitions of terms in documents that are incorporated by reference herein conflict with those used herein, the definitions used herein govern.

The techniques and procedures described or referenced herein are generally well understood and commonly employed using conventional methodology by those skilled in the art, such as, for example, the widely utilized methodologies described in Sambrook et al., MOLECULAR CLONING: A LABORATORY MANUAL (3rd. ed.) (2001) (Cold Spring Harbor Laboratory Press, Cold Spring Harbor, N.Y.); CURRENT PROTOCOLS IN MOLECULAR BIOLOGY (F. M. Ausubel, et al. eds.) (2003); the series METHODS IN ENZYMOLOGY (Academic Press, Inc.): PCR 2: A PRACTICAL APPROACH (M. J. MacPherson, B. D. Hames and G. R. Taylor eds.) (1995), ANTIBODIES, A LABORATORY MANUAL (Harlow and Lane, eds.) (1988), and ANIMAL CELL CULTURE (R. I. Freshney, ed.) (1987); OLIGONUCLEOTIDE SYNTHESIS (M. J. Gait, ed.) (1984); METHODS IN MOLECULAR BIOLOGY (Humana Press); CELL BIOLOGY: A LABORATORY NOTEBOOK (J. E. Cellis, ed.) (1998) (Academic Press); INTRODUCTION TO CELL AND TISSUE CULTURE (J. P. Mather and P. E. Roberts) (1998) (Plenum Press); CELL AND TISSUE CULTURE LABORATORY PROCEDURES (A. Doyle, J. B. Griffiths, and D. G. Newell, eds.) (1993-8) (J. Wiley and Sons); HANDBOOK OF EXPERIMENTAL IMMUNOLOGY (D. M. Weir and C. C. Blackwell, eds); GENE TRANSFER VECTORS FOR MAMMALIAN CELLS (J. M. Miller and M. P. Calos, eds.) (1987); PCR: THE POLYMERASE CHAIN REACTION (Mullis et al., eds.) (1994); CURRENT PROTOCOLS IN IMMUNOLOGY (J. E. Coligan et al., eds.) (1991); SHORT PROTOCOLS IN MOLECULAR BIOLOGY (1999) (Wiley and Sons); IMMUNOBIOLOGY (C. A. Janeway and P. Travers) (1997); ANTIBODIES (P. Finch) (1997); ANTIBODIES: A PRACTICAL APPROACH (D. Catty., ed.) (1988-1989) (IRL Press); MONOCLONAL ANTIBODIES: A PRACTICAL APPROACH (P. Shepherd and C. Dean, eds.) (2000) (Oxford University Press); USING ANTIBODIES: A LABORATORY MANUAL (E. Harlow and D. Lane (1999) (Cold Spring Harbor Laboratory Press); THE ANTIBODIES (M. Zanetti and J. D. Capra, eds.) (1995) (Harwood Academic Publishers); and updated versions thereof.

### Definitions

Unless otherwise defined herein, scientific, and technical terms used in connection with the present invention have the meanings that are commonly understood by those of ordinary skill in the art.

As used herein, the use of the word “a” or “an” or “the” when used in conjunction with the terms “comprising,” “including,” “having,” or “containing,” or variations of these terms, may mean “one,” but it is also consistent with the meaning of “one or more,” “at least one,” and “one or more than one.” For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

Any example(s) following the term “e.g.” or “for example” is not meant to be exhaustive or limiting.

As used herein, the term “about” and “approximately” and “substantially” when used to modify a numerically defined parameter (e.g., the dose of “X”) means that the parameter may vary by as much as 10% below or above the stated numerical value for that parameter. Therefore, in any disclosed aspect, the terms may be substituted with “within [a percentage] of” what is specified. In one non-limiting aspect, the percentage includes 0.1, 0.5, 1, 5, and 10 percent. For example, a dose of about 5 mg means 5 mg±10%, i.e., it may vary between 4.5 mg and 5.5 mg.

Recitation of ranges of values herein is merely intended to serve as a shorthand method of referring individually to each separate value falling within the range. Unless otherwise indicated herein, each individual value is incorporated into the specification as if it was individually recited herein.

The phrase “essentially all” is defined as “at least 95%”; if essentially all members of a group have a certain property, then at least 95% of members of the group have that property. In some aspects, essentially all means equal to any one of, at least any one of, or between (inclusive or exclusive) any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. As a result, the compositions and methods of the present disclosure that “comprise,” “have,” “include” or “contain” one or more elements possesses those one or more elements but are not limited to possessing only those one or more elements. Likewise, an element of a composition or method of the present disclosure that “comprises,” “has,” “includes” or “contains” one or more features possesses those one or more features but is not limited to possessing only those one or more features.

It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” “a further aspect,” “one embodiment,” “an embodiment,” “a particular embodiment,” “a related embodiment,” “a certain embodiment,” “an additional embodiment,” “a further embodiment,” or combinations thereof means that a particular feature, structure or characteristic described in connection with the aspect is included in at least one aspect or embodiment of the present disclosure. Thus, the appearances of the foregoing phrases in various places throughout this specification are not necessarily all referring to the same aspect or embodiment. Furthermore, the particular features, structures, and/or characteristics may be combined in any suitable manner in one or more aspects or embodiments.

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g., monospecific, bispecific), and includes portions of intact antibodies that retain the ability to bind to a given antigen (e.g., an “antigen-binding fragment”), and any other modified configuration of an immunoglobulin molecule that comprises an antigen binding site.

An antibody includes an antibody of any class, such as IgG, IgA, or IgM (or subclass thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known. It is contemplated that any one or more of the foregoing immunoglobulins or subclasses thereof may be excluded from an embodiment.

Examples of antibody antigen-binding fragments and modified configurations include (i) a Fab fragment (a monovalent fragment consisting of the VL, VH, CL and CH1 domains); (ii) a F(ab′) 2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see, e.g., Bird et al., Science 1988; 242:423-426 and Huston et al., Proc. Natl. Acad. Sci. 1988 USA 85:5879-5883. Other forms of single chain antibodies, such as diabodies, are also encompassed. It is contemplated that any one or more of the foregoing antibody antigen-binding fragments or modified configurations thereof may be excluded from an embodiment.

In addition, further encompassed are antibodies that are missing a C-terminal lysine (K) amino acid residue on a heavy chain polypeptide (e.g., human IgG₁ heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine. Accordingly, the present disclosure includes compositions comprising an anti-BMP9 antibody with a heavy chain lacking the C-terminal lysine residue. In some embodiments, the present disclosure includes compositions comprising an anti-BMP9 antibody with a heavy chain having a C-terminal lysine residue and an anti-BMP9 antibody with a heavy chain lacking the C-terminal lysine residue. In some embodiments, the present disclosure includes compositions comprising an anti-BMP9 antibody lacking the C-terminal lysine residue.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs), also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J. Mol. Biol. 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc. Natl. Acad. Sci. (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, J. Biol. Chem., 283:1156-1166. Still other CDR boundary definitions may not strictly follow one of the above approaches but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, and/or conformational definitions. It is contemplated that any one or more of these definitions may be excluded in an embodiment.

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL).

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g., a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally, the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG₁ heavy chain constant region. “Fc chain” may refer to this polypeptide in isolation, or in the context of a larger molecule (e.g., in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions. A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification. It is contemplated that any one or more of the foregoing Fc chains or domains may be excluded from an embodiment.

A “monoclonal antibody” (mAb) refers to an antibody that is derived from a single copy or clone, including, e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “human antibody” refers to an antibody which possesses an amino acid sequence that corresponds to that of an antibody produced by a human or has been made using any technique for making fully human antibodies. For example, fully human antibodies may be obtained by using commercially available mice that have been engineered to express specific human immunoglobulin proteins, or by library (e.g., phage, yeast, or ribosome) display techniques for preparing fully human antibodies. This definition of a human antibody specifically excludes a humanized antibody comprising non-human antigen binding residues.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a non-human (e.g., murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences but are included to further refine and optimize antibody performance.

It is contemplated that any one or more of the antibody types described herein may be excluded from an embodiment.

An “antigen” refers to the molecular entity used for immunization of an immunocompetent vertebrate to produce the antibody that recognizes the antigen or to screen an expression library (e.g., phage, yeast, or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, USING ANTIBODIES, A LABORATORY MANUAL, Cold Spring Harbor Laboratory Press, Cold Spring Harbor, New York, 1999. In addition, or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level, the epitope can be characterized by the amino acid residues that it comprises as defined by a specific criterion, e.g., by distance between atoms (e.g., heavy, i.e., non-hydrogen, atoms) in the antibody and the antigen. At a further less detailed level, the epitope can be characterized through function, e.g., by competition binding with other antibodies. The epitope can also be defined more generically as comprising amino acid residues for which substitution by another amino acid will alter the characteristics of the interaction between the antibody and antigen (e.g., using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies is mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K D). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE™ system. BIACORE™ kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (OCTET® QKe system, ForteBio, now Sartorius). Alternatively, or in addition, a KINEXA® (Kinetic Exclusion Assay) assay, available from Sapidyne Instruments (Boise, ID) can also be used.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

A “bispecific antibody” refers to a molecule that has binding specificity for at least two different epitopes. In some embodiments, bispecific antibodies can bind simultaneously two different antigens. In other embodiments, the two different epitopes may reside on the same antigen.

The term “half maximal effective concentration (EC₅₀)” refers to the concentration of a therapeutic agent which causes a response halfway between the baseline and maximum after a specified exposure time. EC₉₀ refers to the concentration of a therapeutic agent corresponding to 90% of the maximum possible response after a specified exposure time. The therapeutic agent may cause inhibition or stimulation. The EC₅₀ value is commonly used, and is used herein, as a measure of potency.

The term “half maximal inhibitory concentration” (IC₅₀) refers to the concentration of the therapeutic agent that is required for 50% inhibition of the maximum response obtained.

An “agonist” refers to a substance which promotes (i.e., induces, causes, enhances, and/or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, and/or reduces a biological activity or effect of another molecule, such as a receptor. The term antagonist encompasses substances (such as an antibody) which bind to a molecule to prevent or reduce the activity of that molecule.

The term “compete,” as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present disclosure. Regardless of the mechanism by which such competition or cross-competition occurs (e.g., steric hindrance, conformational change, and/or binding to a common epitope, or portion thereof), the skilled artisan would appreciate, based upon the teachings provided herein, that such competing or cross-competing antibodies are encompassed and can be useful for the methods disclosed herein.

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol. 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

The term “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using an in vitro ADCC assay, but other assays or methods for determining ADCC activity, e.g., in an animal model etc., are contemplated. In some embodiments, an antibody with enhanced ADCC activity has enhanced affinity for FcgRIIIA.

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

The term “complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 1996; 202: 163, may be performed. Antibodies with altered Fc region amino acid sequences and increased or decreased C1q binding capability are described, e.g., in U.S. Pat. Nos. 6,194,551, 7,923,538, 7,994,290, and WO 1999/51642.

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, and/or deliberate mutation. A host cell includes cells transfected in vivo with a polynucleotide(s) disclosed herein.

A “vector” refers to a construct, which is capable of delivering, and, preferably, expressing, one or more gene(s) or sequence(s) of interest (e.g., an antibody-encoding gene) in a host cell. Examples of vectors include, but are not limited to plasmids and viral vectors, and may include naked nucleic acids, or may include nucleic acids associated with delivery-aiding materials (e.g., cationic condensing agents, liposomes, etc.). Vectors may include DNA or RNA. An “expression vector” as used herein refers to a vector that includes at least one polypeptide-encoding gene, at least one regulatory element (e.g., promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers, and terminator). For translation, one or more translational controlling elements may also be included such as ribosome binding sites, translation initiation sites, and stop codons. It is contemplated that any one or more of these foregoing components or elements may be excluded in an embodiment.

An “isolated” molecule (e.g., antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, and/or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “polynucleotide” or “nucleic acid,” (used interchangeably herein) refers to a chain of nucleotides of any length and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. It is contemplated that any one or more of these foregoing modifications may be excluded in an embodiment.

A “conservative substitution” refers to replacement of one amino acid by a biologically, chemically, and/or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g., algorithms), which are well known in the art.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

The term “excipient” refers to any material which, which combined with an active ingredient of interest (e.g., antibody), allow the active ingredient to retain biological activity. The choice of excipient will to a large extent depend on factors such as the mode of administration, the effect of the excipient on solubility and stability, and the nature of the dosage form. As used herein, “excipient” includes any and all solvents, dispersion media, coatings, antibacterial and antifungal agents, isotonic and absorption delaying agents, carriers, diluents, and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, and/or polyalcohols such as mannitol, or sorbitol in the composition. It is contemplated that any one or more of these foregoing excipients may be excluded in an embodiment.

The terms “treating,” “treat,” or “treatment” refer to any type of treatment, e.g., such as to relieve, alleviate, ameliorate, and/or slow the progression of the patient's disease, disorder, or condition, or any tissue damage associated with the disease. In some embodiments, the disease, disorder, or condition is hypertension, optionally, pulmonary arterial hypertension.

The terms “prevent” or “prevention” refer to one or more of delay of onset, reduction in frequency, and/or reduction in severity of at least one sign or symptom (e.g., shortness of breath, fatigue, dizziness, chest pressure or pain, swelling (edema), cyanosis, heart palpitations, ventricle hypertrophy, and/or diminishing functioning of the right portion of the heart (leading to heart failure) for pulmonary arterial hypertension) of a particular disease, disorder, or condition (e.g., pulmonary arterial hypertension). In some embodiments, prevention is assessed on a population basis such that an agent is considered to “prevent” a particular disease, disorder, or condition if a statistically significant decrease in the development, frequency, and/or intensity of one or more symptoms of the disease, disorder, or condition is observed in a population susceptible to the disease, disorder, or condition. Prevention may be considered complete when onset of disease, disorder, or condition has been delayed for a predefined period of time.

The terms “subject,” “individual,” or “patient” (used interchangeably herein) refer to any animal, including mammals. Mammals according to embodiments include canine, feline, bovine, caprine, equine, ovine, porcine, rodents, lagomorphs, primates, humans, and the like, and encompass mammals in utero. In an embodiment, humans are suitable subjects. Human subjects may be of any gender and at any stage of development. In some embodiments, a subject is a patient with disease hypertension, optionally, pulmonary arterial hypertension.

The term “therapeutically effective amount” refers to the amount of active ingredient that elicits the biological or medicinal response in a tissue, system, animal, individual, and/or human that is being sought by a researcher, veterinarian, medical doctor, and/or other clinician, which may include one or more of the following:
(1) preventing the disease; for example, preventing a disease, condition, or disorder in an individual that may be predisposed to the disease, condition, or disorder but does not yet experience and/or display the pathology and/or symptomatology of the disease;
(2) inhibiting the disease; for example, inhibiting a disease, condition, or disorder in an individual that is experiencing and/or displaying the pathology and/or symptomatology of the disease, condition, or disorder (i.e., arresting and/or slowing further development of the pathology and/or symptomatology); and
(3) ameliorating the disease; for example, ameliorating a disease, condition, or disorder in an individual that is experiencing and/or displaying the pathology and/or symptomatology of the disease, condition, or disorder (i.e., reversing the pathology and/or symptomatology).

### Antibodies to BMP9

The disclosure provides antibodies that bind to bone morphogenetic protein-9 (BMP9). BMP9, also known as growth differentiation factor 2 (GDF2), is encoded in humans by the GDF2 gene. It belongs to the transforming growth factor beta (TGFβ) superfamily. BMPs are synthesized as large precursors of about 400-500 amino acids consisting of an N-terminal signal peptide directing secretion, a prodomain for proper folding and a C-terminal mature peptide (Bragdon B. Moseycbuk O. Saidanha S. King D, Julian J. Nobe A (2011) Bone inorphogenetic proteins: a critical review. Cell Signal 23:609-620). Upon dimerization, the carboxy-terminal mature proteins are cleaved from the prodomains (Nelsen S M, Christian J L (2009) Site-specific cleavage of MIN by furin, PC6, and PC7. J. Biol. Chem. 284:27157-27166). Following cleavage, the prodomain remains non-covalently associated with the mature active BMP dimer. Three type II receptors [bone morphogenetic protein receptor type II (BMPRII), activin receptor type 2A (ActRIIA), and type 2B (ActRIIB)], and four type I receptors [activin receptor-like kinase (ALK1, ALK2, ALK3, and ALK6)] are involved in BMP signaling. Following ligand-induced receptor hetero-oligomerization, type I receptors are trans-phosphorylated and activated by type II receptors. These activated receptor complexes phosphorylate the receptor-regulated Smads (R-Smads: Smad1, Smad5, and Smad8), enabling the formation of a complex with the co-Smad, Smad4. R-Smad/Smad4 complexes translocate into the nucleus and regulate target gene expression by binding to regulatory elements and recruiting transcriptional co-repressor and/or activation complexes on their promoters (Bidart, M., Ricard, N., Levet, S. et al. BMP9 is produced by hepatocytes and circulates mainly in an active mature form complexed to its prodomain. Cell. Mol. Life Sci. 69,313-324 (2012)).

As used herein, the term bone morphogenetic protein-9 or bone morphogenetic protein 9 (BMP9), also known as Growth differentiation factor 2 (GDF2) includes variants, isoforms, homologs, orthologs, and paralogs of BMP9. In some embodiments, an antibody disclosed herein cross-reacts with BMP9 from species other than human, such as BMP9 of cynomolgus monkey, and/or rat, as well as different forms of BMP9. In some embodiments, an antibody may be completely specific for human BMP9 and may not exhibit species cross-reactivity (e.g., does not bind mouse BMP9). In some embodiments, an antibody may be completely specific for human BMP9 but does not exhibit cross-reactivity to other transforming growth factor beta superfamily members (e.g., does not bind TGFβ1, GDF8, GDF9, GDF11, Activin A, and BMP10). As used herein, the term BMP9 refers to naturally occurring human BMP9 unless contextually dictated otherwise. Therefore, an “BMP9 antibody,” “anti-BMP9 antibody,” or other similar designation means any antibody (as defined herein) that binds or reacts with BMP9, or an isoform, fragment, and/or derivative thereof. The full length, human BMP9, as represented by UniProtKB/Swiss-Prot accession number Q9UK05 is herein provided as SEQ ID NO: 78. The full length, mature form of human BMP9 (monomer) is herein provided as SEQ ID NO: 79.

Without wishing to be bound by any particular theory, perturbation of the BMP9 signaling pathway has emerged as essential in endothelial (dys)function and vascular remodeling, in particular, in pulmonary arterial hypertension. Suppression of BMP9 in different complementary and well-established animal models of PAH has been shown to partially protect against experimental pulmonary hypertension (Tu L., Desroches-Castan A., Mallet C., et al. Selective BMP-9 inhibition partially protects against experimental pulmonary hypertension. Circulation Research. 2019; 124:846-855; Bouvard C., Tu L., Rossi M., et al. Different cardiovascular and pulmonary phenotypes are observed in single- and double-knock-out mice deficient in BMP9 and/or BMP10. Cardiovasc Res. 2021; June 4: cvab87).

A neutralizing or “blocking” antibody refers to an antibody whose binding to BMP9 one or both of (i) interferes with, limits, and/or inhibits the interaction between BMP9 and a type I receptor or a type II receptor or both type I and type II receptors, such as (ALK1, ALK2, ALK3 ALK6, BMPRII, ActRIIA and ActRIIB); and/or (ii) results in inhibition of at least one biological function of BMP9 binding. Assays to determine neutralization by an antibody of the disclosure are well-known in the art.

“Biological function” or “biological activity” of BMP9 is meant to include binding of BMP9 to endothelial cells, induction of phospho-SMAD1/5/9 nuclear translocation in endothelial cells, and induction of phospho-SMAD2 nuclear translocation in endothelial cells. The biological function or biological activity of BMP9 can, but need not be, mediated by the interaction between BMP9 and its receptors.

In some embodiments, an anti-BMP9 antibody of the disclosure encompasses an antibody that one or both of i) competes for binding to human BMP9 with, and/or ii) binds the same epitope as, an antibody having the amino acid sequence of a heavy chain variable region set forth as SEQ ID NO: 77 and the amino acid sequence of a light chain variable region set forth as SEQ ID NO: 74.

Anti-BMP9 antibodies of the present disclosure can encompass monoclonal antibodies, polyclonal antibodies, antibody fragments (e.g., Fab, Fab′, F(ab′) 2, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-BMP9 antibody is a monoclonal antibody. In some embodiments, an anti-BMP9 antibody is a human or humanized antibody. In some embodiments, an anti-BMP9 antibody is a chimeric antibody. It is contemplated that any one or more of these foregoing antibody types may be excluded in an embodiment.

In some embodiments, disclosed is an antibody having a light chain variable region (VL) sequence and a heavy chain variable region (VH) sequence as found in Table 3/Table 4, or variants thereof. In Table 4, the underlined sequences are CDR sequences (Kabat definition), and the sequences in italics are CDR sequences (Chothia definition). In Table 1, the K D indicates affinity for human BMP9 as measured using surface plasmon resonance at 37° C., unless indicated otherwise.

Tables 3 and 4 provide VH and VL sequences for mAbs of particular embodiments of the present disclosure.

Embodiments also provide CDR portions of antibodies to BMP9. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, J. Biol. Chem., 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, and/or contact CDRs. In other words, in embodiments with more than one CDR, the CDRs may be any of Kabat, Chothia, extended, AbM, conformational, contact CDRs, or combinations thereof. It is contemplated that any one or more of these foregoing CDRs may be excluded in an embodiment.

Table 4 provides examples of CDR sequences of anti-BMP9 antibodies provided herein. In some embodiments, the antibody comprises three CDRs of any one of the heavy chain variable regions shown in Table 4. In some embodiments, the antibody comprises three CDRs of any one of the light chain variable regions shown in Table 4. In some embodiments, the antibody comprises three CDRs of any one of the heavy chain variable regions shown in Table 4, and three CDRs of any one of the light chain variable regions shown in Table 4. In some embodiments, the antibody comprises three light chain CDRs and three heavy chain CDRs from Table 4.

In some embodiments, the antibody comprises one or both of i) the full-length heavy chain, with or without the C-terminal lysine, and/or ii) the full-length light chain of an anti-BMP9 antibody (e.g., Ab1076, Ab93, Ab352, Ab804, Ab101, Ab732, Ab100 and Ab89). The amino acid sequences of the full-length heavy chain and light chain for antibodies BMP9 are shown below in Table 4.

In certain embodiments, an antibody described herein comprises an Fc domain. The Fc domain can be derived from IgA (e.g., IgA₁ or IgA₂), IgD, IgE, IgM, or IgG (e.g., IgG₁, IgG₂, IgG₃, or IgG₄). In some embodiments, an anti-BMP9 antibody is an IgG antibody.

Embodiments encompass modifications to the variable regions, the CDR regions, and/or the heavy chain and light chain sequences shown in Tables 4 and 5. For example, embodiments include antibodies comprising functionally equivalent variable regions and CDRs that do not significantly affect their properties, as well as variants having enhanced or decreased activity or affinity. For example, the amino acid sequence may be mutated to obtain an antibody with the desired binding affinity to BMP9. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs. It is contemplated that any one or more of these foregoing modifications may be excluded in an embodiment.

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, and/or to alter such properties as complement fixation, FcR binding and antibody-dependent cell-mediated cytotoxicity. In some embodiments, no more than one to five (e.g., at least, at most, or exactly 1, 2, 3, 4, or 5) conservative amino acid substitutions are made within the framework region or constant region. In other embodiments, no more than one to three (e.g., at least, at most, or exactly 1, 2, or 3) conservative amino acid substitutions are made within the framework region or constant region. According to certain embodiments, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), does not activate microglia, and/or has reduced activities (compared to the unmodified antibody) in any one or more of the following: triggering complement mediated lysis, stimulating ADCC, and/or activating microglia. It is contemplated that any one or more of these foregoing characteristics due to a modified constant region may be excluded in an embodiment. Different modifications of the constant region may be used to achieve optimal level or combination of effector functions. See, e.g., Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-BMP9 antibodies containing variations of the variable regions, the CDRs, or heavy chain and light chain sequences shown in Tables 4 and 5, wherein such variant polypeptides share at least, at most, exactly, between (inclusive or exclusive) any two of, or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or any range thereof, amino acid sequence identity to any of the amino acid sequences disclosed in Tables 4 or 5. For example, in some embodiments, the disclosure provides anti-BMP9 antibodies containing variations of the variable regions, the CDRs, or heavy chain and light chain sequences shown in Tables 4 and 5, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in Tables 4 or 5. These amounts are not meant to be limiting and increments between the recited percentages are specifically envisioned as part of the disclosure. It is contemplated that any one or more of these foregoing percentages may be excluded in an embodiment.

It is contemplated that a region or fragment of a polypeptide of the disclosure may have an amino acid sequence that has, has at least or has at most 1, 2, 3, 4, 5, 6, 7, 8, 9, or 10 amino acid substitutions, contiguous amino acid additions, and/or contiguous amino acid deletions with respect to any of SEQ ID NOs: 1-77, 80-84, or 89. In some embodiments, an anti-BMP9 antibody, or antigen-binding fragment thereof, comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range thereof) identical to any of SEQ ID NOs: 1-77, 80-84, or 89. It is contemplated that any one or more of these foregoing percentages may be excluded in an embodiment.

Moreover, in some embodiments, a region, or fragment comprises an amino acid region of at least, at most, exactly, or between (inclusive or exclusive) any two of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids (or any range thereof) starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOS: 1-77, 80-84, or 89 (where position 1 is at the N-terminus of the SEQ ID NO).

An anti-BMP9 antibody, or antigen-binding fragment thereof, of the disclosure may include at least, at most, exactly, or between (inclusive or exclusive) any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more (or any range thereof) variant amino acids or amino acid substitutions. In some embodiments, a variant or amino acid substitution is at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and/or 500 in any of SEQ ID NOS: 1-77, 80-84, or 89 (where position 1 is at the N-terminus of the SEQ ID NO), and the anti-BMP9 antibody, or antigen-binding fragment thereof may be at least, at most, exactly, or between (inclusive or exclusive) any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 600, or more contiguous amino acids, or any range thereof, of any of SEQ ID NOs: 1-77, 80-84, or 89. It is contemplated that any one or more of these foregoing percentages may be excluded in an embodiment.

In some embodiments, the disclosure provides anti-BMP9 antibodies containing variations of the variable regions shown in Tables 4 and 5. In some embodiments, the VH comprises the amino acid sequence of SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, or a variant thereof comprising one to four amino acid substitutions at residues that are not within a CDR. In some embodiments, the one to four (i.e., 1, 2, 3, or 4) amino acid substitutions may be selected from the group consisting of K13Q, D73N, S74A, T77S, K83R, and T84A of SEQ ID NO: 27. In some embodiments, the VL comprises the amino acid sequence of SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74, or a variant thereof comprising one to four (i.e., 1, 2, 3, or 4) amino acid substitutions at residues that are not within a CDR. In some embodiments, the one to four amino acid substitutions may be selected from the group consisting of M43T, V76S, Q79R, G84A, and Y85D of SEQ ID NO: 22. In some embodiments, provided herein is an anti-BMP9 antibody that comprises a VH and a VL, wherein the antibody VH has an amino acid sequence encoded by the nucleic acid sequence of the insert of the plasmid deposited with the ATCC having ATCC Accession No. PTA-127292 and/or the antibody VL has an amino acid sequence encoded by the nucleic acid sequence of the insert of the plasmid deposited with the ATCC having ATCC Accession No. PTA-127293.

Embodiments also encompass fusion proteins comprising one or more components of the antibodies disclosed herein. In some embodiments, a fusion protein may be made that comprises all or a portion of an anti-BMP9 antibody disclosed herein linked to another polypeptide. In another embodiment, only the variable domains of the anti-BMP9 antibody are linked to the polypeptide. In another embodiment, the VH domain of an anti-BMP9 antibody is linked to a first polypeptide, while the VL domain of an anti-BMP9 antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Biological Activity of Anti-BMP9 Antibodies

In addition to binding an epitope on BMP9, an antibody of the disclosure can mediate a biological activity. That is, the disclosure includes an isolated antibody that specifically binds BMP9 and mediates at least one detectable activity selected from the following:
(i) binds specifically to human BMP9;
(ii) binds specifically to cynomolgus monkey (cyno) BMP9;
(iii) binds specifically to rat BMP9;

(iv) one or more of reduces, inhibits, and/or neutralizes the binding of BMP9 to type II receptor (BMPRII, ActRIIA, and/or ActRIIB) (e.g., Ab89, Ab93, Ab100, Ab101);
(v) one or more of reduces, inhibits, and/or neutralizes the binding of BMP9 to type II receptor (BMPRII, ActRIIA, and/or ActRIIB) and weakly inhibits the binding of BMP9 to type I receptor (ALK1) (e.g., Ab352, Ab804 and Ab1076);
(vi) one or more of reduces, inhibits, and/or neutralizes the binding of BMP9 to endothelial cells;
(vii) one or more of reduces, inhibits, and/or neutralizes SMAD1/5/9 phosphorylation and/or phospho-SMAD1/5/9 nuclear translocation in endothelial cells;
(viii) one or more of reduces, inhibits, and/or neutralizes SMAD2 phosphorylation or phospho-SMAD2 nuclear translocation in endothelial cells;
(ix) reduces right ventricular systolic pressure;
(x) reduces right ventricular hypertrophy;
(xi) reduces pulmonary arterial pressure (e.g., by at least, at most, exactly, between (inclusive or exclusive) any two of, or about 1 mmHg, 2 mmHg, 3 mmHg, 4 mmHg, 5 mmHg, 6 mmHg, 7 mmHg, 8 mmHg, 9 mmHg, 10 mmHg, 11 mmHg, 12 mmHg, 13 mmHg, 14 mmHg, 15 mmHg, 16 mmHg, 17 mmHg, 18 mmHg, 19 mmHg, 20 mmHg, 21 mmHg, 22 mmHg, 23 mmHg, 24 mmHg, or 25 mmHg, or any range thereof) (e.g., at least 3 mmHg, at least 5 mmHg, at least 7 mmHg, at least 10 mmHg, at least 12 mmHg, at least 15 mmHg, at least 20 mmHg, or at least 25 mmHg);
(xii) reduces pulmonary vascular resistance;
(xiii) increases cardiac output or cardiac index;
(xiv) increases pulmonary capillary wedge pressure;
(xv) increase left ventricular end-diastolic pressure;
(xvi) increases exercise capacity of the subject;
(xvii) increases the subject's 6-minute walk distance (e.g., by at least, at most, exactly, between (inclusive or exclusive) any two of, or about 10, 15, 20, 25, 30, 35, 40, 45, 50, 55, 60, 65, 70, 75, 80, 85, 90, 95, or 100 meters, or any range thereof) (e.g., by at least 10, 20, 30, 40, 50, 60, 70, 80, 90, 100 or more meters); and
(xviii) reduces subject's Borg dyspnea index (BDI) (e.g., Clini Enrico et al. Multidiscip. Respir. Med. 2010; 5(3):202-210).
   It is contemplated that any one or more of these foregoing activities may be excluded in an embodiment.

Assays to measure the functional activities described above are well known in the art. For example, binding activity can be measured using an enzyme-linked immunosorbent assay (ELISA), radioimmunoassay (REA), FACS analysis, bioassay (e.g., growth inhibition), Western Blot assay, BIACORE™ assay, and/or by surface plasmon resonance. Each of these assays generally detects the presence of protein-antibody complexes of particular interest by employing a labeled reagent (e.g., an antibody) specific for the complex of interest.

The capacity to block or compete with the reference molecule (e.g., a receptor or antibody) may be determined by, e.g., a competition binding assay. With a competition binding assay, the antibody under test is examined for ability to inhibit specific binding of the reference molecule to a common antigen, such as BMP9 polypeptide. A test antibody competes with the reference molecule for specific binding to the antigen if an excess of the test antibody substantially inhibits binding of the reference molecule. Substantial inhibition means that the test antibody reduces specific binding of the reference molecule usually by at least, at most, exactly, between (inclusive or exclusive) any two of, or about 10%, 15%, 20%, 25%, 30%, 35%, 40%, 45%, 50%, 55%, 60%, 65%, 70%, 75%, 80%, 85%, or 90%, or any range thereof (e.g., at least 10%, 25%, 50%, 75%, or 90%). There are a number of known competition binding assays that can be used to assess competition of an antibody with a reference antibody for binding to a particular protein, in this case, BMP9. These include, e.g., solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see Harlow & Lane, supra); solid phase direct label RIA using 1-125 label (see Morel et al., Molec. Immunol. 25:7-15, 1988); solid phase direct biotin-avidin EIA (Cheung et al., Virology 176:546-552, 1990); and direct labeled RIA (Moldenhauer et al., Scand. J. Immunol. 32:77-82, 1990).

BMP9-binding antibodies and antigen-binding fragments thereof of the present disclosure can be further tested for reactivity with BMP9 polypeptide or antigenic fragment by Western blotting. Briefly, purified BMP9 polypeptides or fusion proteins, and/or cell extracts from cells expressing BMP9, can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Examples of functional assays are also described in the Example section below. It is contemplated that any one or more of these foregoing functional assays may be excluded in an embodiment.

In some embodiments, the antibody that mediates at least one of the functional activities described herein is selected from the group consisting of Ab89, Ab93, Ab100, Ab101, Ab352, Ab732, Ab804, and Ab1076. In some embodiments, the antibody is Ab93, Ab352, Ab804, or Ab1076. In some embodiments, the antibody is Ab1076. In some embodiments, the antibody is Ab101. In some embodiments, the antibody is Ab732. In some embodiments, the antibody is Ab101 or Ab732. It is contemplated that any one or more of these foregoing antibodies may be excluded in an embodiment.

In some embodiments, the antibody binds human BMP9 with an affinity of about 15 pM to about 80 pM at 37° C. as measured by surface plasmon resonance. In some embodiments, the antibody binds human BMP9 with an affinity of about 15 pM to about 150 pM at 37° C. as measured by surface plasmon resonance. In some embodiments, the antibody binds human BMP9 with an affinity of at least, at most, exactly, between (inclusive or exclusive) any two of, or about 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, or 150 pM, or any range thereof (e.g., about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, or about 150 pM) at 37° C. as measured by surface plasmon resonance. In some embodiments, the antibody binds human BMP9 with an affinity of about 87 pM at 37° C. as measured by surface plasmon resonance. In some embodiments, the antibody binds human BMP9 with an affinity of about 87.41 pM or about 90 pM at 37° C. as measured by surface plasmon resonance. In some embodiments, the antibody is Ab1076.

### Polynucleotides Encoding Anti-BMP9 Antibodies, and Methods of Manufacture

The disclosure also provides polynucleotides encoding any of the antibodies of the present disclosure, including antibody portions and modified antibodies described herein. Embodiments also provide a method of making any of the antibodies and polynucleotides described herein. Polynucleotides can be made and proteins expressed therefrom by procedures known in the art.

If desired, an anti-BMP9 antibody of interest may be sequenced, and the polynucleotide sequence may then be cloned into a vector for expression or propagation. The sequence encoding the antibody of interest may be maintained in a vector in a host cell, and the host cell can then be expanded and frozen for future use. Production of recombinant monoclonal antibodies in cell culture can be carried out through cloning of antibody genes from B cells by means known in the art. See, e.g., Tiller et al., 2008, J. Immunol. Methods 329, 112; U.S. Pat. No. 7,314,622.

In some embodiments, provided herein is a polynucleotide comprising a sequence encoding one or both of the heavy chain and/or the light chain variable regions of an anti-BMP9 antibody provided herein. The sequence encoding the antibody of interest may be maintained in a vector in a host cell, and the host cell can then be expanded and frozen for future use. Vectors (including expression vectors) and host cells are further described herein.

In some embodiments, the disclosure provides polynucleotides encoding the amino acid sequences of any of the following anti-BMP9 antibodies: Ab89, Ab93, Ab100, Ab101, Ab352, Ab732, Ab804, and Ab1076. In one embodiment, the disclosure provides polynucleotides encoding the amino acid sequence of anti-BMP9 antibody Ab93, Ab1076, Ab101, or Ab732. In one embodiment, the disclosure provides polynucleotides encoding the amino acid sequence of anti-BMP9 antibody Ab1076.

In some embodiments, the disclosure provides polynucleotides encoding one or more anti-BMP9 antibody heavy chain polypeptides comprising an amino acid sequence selected from the group consisting of: SEQ ID NOs: 8, 23, 35, 44, 52, 60, 69, and 75.

In some embodiments, the disclosure provides polynucleotides encoding one or more anti-BMP9 antibody light chain polypeptides comprising an amino acid sequence selected from the group consisting of: SEQ ID NOs: 1, 18, 29, 40, 49, 56, 64, and 72.

In some embodiments, the disclosure provides polynucleotides comprising SEQ ID NO: 87. In some embodiments, the disclosure provides polynucleotides comprising SEQ ID NO: 88.

In some embodiments, the disclosure provides polynucleotides encoding one or more anti-BMP9 antibody VH polypeptides comprising an amino acid sequence selected from the group consisting of: SEQ ID NOs: 16, 27, 39 48, 55, 63, 71, and 77.

In some embodiments, the disclosure provides polynucleotides encoding one or more anti-BMP9 antibody VL polypeptides comprising an amino acid sequence selected from the group consisting of: SEQ ID NOs: 6, 22, 33, 43, 51, 59, 68, and 74.

In some embodiments, the disclosure provides polynucleotides comprising SEQ ID NO: 85. In some embodiments, the disclosure provides polynucleotides comprising SEQ ID NO: 86.

The present disclosure provides a polypeptide comprising the amino acid sequence encoded by the DNA insert of the plasmid deposited with the ATCC and having Accession No. PTA-127292, encoding the VH domain of antibody Ab1076. The present disclosure further provides a polypeptide comprising the amino acid sequence encoded by the insert of the plasmid deposited with the ATCC and having Accession No. PTA-127293 encoding the VL domain of antibody Ab1076.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions, and/or substitutions of nucleotides. The resulting mRNA and protein may, but need not, have an altered structure or function. Alleles may be identified using standard techniques (such as hybridization, amplification, and/or database sequence comparison).

In one embodiment, the VH and/or VL domains, and/or full-length HC and/or LC, are encoded by separate polynucleotides. Alternatively, both VH and/or VL, and/or HC and/or LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA, or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating, or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; and suitable transcriptional controlling elements (such as promoters, enhancers, and terminator). For expression (i.e., translation), one or more translational controlling elements are also usually required, such as ribosome binding sites, translation initiation sites, and stop codons. It is contemplated that any one or more of these foregoing vector components or elements may be excluded in an embodiment.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Embodiments also provide host cells comprising any of the polynucleotides described herein. Any host cells capable of over-expressing heterologous DNAs can be used for the purpose of isolating the genes encoding the antibody, polypeptide, or protein of interest. Non-limiting examples of mammalian host cells include but are not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present disclosure. One skilled in the art will appreciate that different cell lines might have different nutrition requirements or might require different culture conditions for optimal growth and polypeptide or protein expression and will be able to modify conditions as needed.

In some embodiments, the heavy and light chain antibodies described herein are produced with a signal peptide (e.g., SEQ ID NO: 89). As used herein, the terms “leader peptide” or “leader sequence” or “leader signal sequence” or “signal sequence” or “signal peptide” (used interchangeably herein) mean any nucleic acid sequence, or amino acid sequence encoded thereby, that may be present on the 5′ end of a nucleic acid molecule and/or at or near the N-terminus of a polypeptide, that when present, may mediate the transport of the polypeptide to an organelle of destination, including, but not limited to, the secretion of the polypeptide from a cell. Such leader sequences include, but are not limited to, nucleic acid sequences encoding SEQ ID NO: 89. Embodiments encompass this and any other leader signals (nucleic and amino acid sequences) known in the art or to be identified which can result in the transport of a polypeptide to the desired organelle, e.g., the endoplasmic reticulum, and/or secreted from the cell. In preferred embodiments, the signal peptide is removed from and/or is not present in the mature polypeptide.

### Pharmaceutical Compositions

In another embodiment, the present disclosure comprises pharmaceutical compositions. A “pharmaceutical composition” refers to a mixture of an antibody and one or more excipients.

Pharmaceutical compositions of this disclosure may be in a variety of forms. These include, for example, liquid, semi-solid, and solid dosage forms, such as liquid solutions (e.g., injectable and infusible solutions), dispersions or suspensions, and lyophilized powders. The form depends on the intended mode of administration and therapeutic application. It is contemplated that any one or more of these foregoing forms may be excluded in an embodiment.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., REMINGTON'S PHARMACEUTICAL SCIENCES, Mack Publishing Co., Easton, Pennsylvania, 1975; Liberman et al., Eds., PHARMACEUTICAL DOSAGE FORMS, Marcel Decker, New York, N.Y., 1980; and Kibbe et al., Eds., HANDBOOK OF PHARMACEUTICAL EXCIPIENTS (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, and/or immunoglobulins; hydrophilic polymers such as polyvinylpyrrolidone; amino acids such as glycine, glutamine, asparagine, histidine, arginine, and/or lysine; monosaccharides, disaccharides, and other carbohydrates including glucose, mannose, and/or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). It is contemplated that any one or more of these foregoing excipients may be excluded in an embodiment.

In some embodiments, a pharmaceutical composition comprises at least, at most, exactly, between (inclusive or exclusive) any two of, or about 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, or 150 mg/mL antibody (or an antigen-binding fragment thereof), or any range thereof (e.g., about 25 mg/mL, 50 mg/mL, 75 mg/mL, 100 mg/mL, 125 mg/mL, 150 mg/mL antibody). In some embodiments, a pharmaceutical composition comprises about 100 mg/mL antibody (or an antigen-binding fragment thereof). In some embodiments, a pharmaceutical composition suitable for SC and/or IV administration comprises about 100 mg/mL anti-BMP9 antibody (or an antigen-binding fragment thereof) (e.g., Ab1076).

In some embodiments, an anti-BMP9 antibody, or antigen-binding fragment thereof, is administered in an intravenous or subcutaneous formulation as a sterile aqueous solution comprising a polypeptide comprising the amino acid sequence of SEQ ID NO: 75. In some embodiments, an anti-BMP9 antibody, or antigen-binding fragment thereof, is administered in an intravenous or subcutaneous formulation as a sterile aqueous solution comprising a polypeptide comprising the amino acid sequence of SEQ ID NO: 72. In some embodiments, an anti-BMP9 antibody, or antigen-binding fragment thereof, is administered in an intravenous or subcutaneous formulation as a sterile aqueous solution comprising a polypeptide comprising the amino acid sequence of SEQ ID NO: 75 and polypeptide comprising the amino acid sequence of SEQ ID NO: 72.

In some embodiments, an anti-BMP9 antibody (or an antigen-binding fragment thereof) (e.g., Ab1076) is administered as an intravenous or subcutaneous formulation that is a sterile aqueous solution containing at least, at most, exactly, between (inclusive or exclusive) any two of, or about 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, or 150 mg/mL antibody, or any range thereof (e.g., about 5 mg/mL, about 10 mg/mL, about 15 mg/mL, about 20 mg/mL, about 25 mg/mL, about 50 mg/mL, about 75 mg/mL, about 100 mg/mL, about 125 mg/mL, or about 150 mg/mL of antibody).

In some embodiments, an intravenous or subcutaneous formulation is a sterile aqueous solution comprising sodium acetate, polysorbate 80, and sodium chloride at a pH ranging from about 5 to 6. In some embodiments, an intravenous or subcutaneous formulation is a sterile aqueous solution comprising at least, at most, exactly, between (inclusive or exclusive) any two of, or about 1 mM, 5 mM, 10 mM, 15 mM, 20 mM, 25 mM, 30 mM, 35 mM, or 40 mM sodium acetate, or any range thereof. In some embodiments, an intravenous or subcutaneous formulation is a sterile aqueous solution comprising 20 mM sodium acetate. In some embodiments, an intravenous or subcutaneous formulation is a sterile aqueous solution comprising at least, at most, exactly, between (inclusive or exclusive) any two of, or about 0.01 mg/mL, 0.05 mg/mL, 0.1 mg/mL, 0.15 mg/mL, 0.2 mg/mL, 0.25 mg/mL, 0.3 mg/mL, 0.35 mg/mL, or 0.4 mg/mL polysorbate 80, or any range thereof. In some embodiments, an intravenous or subcutaneous formulation is a sterile aqueous solution comprising 0.2 mg/mL polysorbate 80. In some embodiments, an intravenous or subcutaneous formulation is a sterile aqueous solution comprising at least, at most, exactly, between (inclusive or exclusive) any two of, or about 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, or 200 mM sodium chloride, or any range thereof. In some embodiments, an intravenous or subcutaneous formulation is a sterile aqueous solution comprising 140 mM sodium chloride. In some aspects, the sodium chloride is at least, at most, exactly, or about pH 5 (e.g., pH 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6, or any range thereof). In some embodiments, an intravenous formulation is a sterile aqueous solution containing 5 or 10 mg/mL of antibody or antigen-binding fragment thereof, with 20 mM sodium acetate, 0.2 mg/mL polysorbate 80, and 140 mM sodium chloride at pH 5.5, optionally, in a nominal fill volume of 1.0 mL. It is contemplated that any one or more of these foregoing ingredients may be excluded in an embodiment.

Further, a solution comprising an antibody, or antigen-binding fragment thereof, can comprise, among many other compounds, glutamic acid, histidine, mannitol, sucrose, trehalose, glycine, poly(ethylene) glycol, EDTA, methionine, polysorbate 80, or any combination thereof, and many other compounds known in the relevant art. It is contemplated that any one or more of these foregoing ingredients may be excluded in an embodiment.

In some embodiments, a pharmaceutical composition comprises at least, at most, exactly, between (inclusive or exclusive) any two of, or about 1 mM, 5 mM, 10 mM, 15 mM, 20 mM, 25 mM, 30 mM, 35 mM, or 40 mM histidine, or any range thereof. In some embodiments, a pharmaceutical composition comprises 20 mM histidine. In some embodiments, a pharmaceutical composition comprises 10 mM histidine. In some embodiments, a pharmaceutical composition comprises at least, at most, exactly, between (inclusive or exclusive) any two of, or about 1%, 2%, 3%, 4%, 5%, 6%, 7%, 8%, 9%, 10%, 11%, 12%, 13%, 14%, 15%, 16%, 17%, 18%, 19%, or 20% sucrose, or any range thereof. In some embodiments, a pharmaceutical composition comprises 8.5% sucrose. In some embodiments, a pharmaceutical composition comprises 5% sucrose. In some embodiments, a pharmaceutical composition comprises at least, at most, exactly, between (inclusive or exclusive) any two of, or about 0.001%, 0.005%, 0.01%, 0.015%, 0.02%, 0.025%, 0.03%, 0.035%, 0.04% polysorbate 80, or any range thereof. In some embodiments, a pharmaceutical composition comprises 0.02% polysorbate 80. In some embodiments, a pharmaceutical composition comprises 0.01% polysorbate 80. In some aspects, the polysorbate 80 is at least, at most, exactly, or about pH 5.8 (e.g., pH 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, or 6.5, or any range thereof). In some embodiments, a pharmaceutical composition comprises at least, at most, exactly, between (inclusive or exclusive) any two of, or about 0.001%, 0.002%, 0.003%, 0.004%, 0.005%, 0.006%, 0.007%, 0.008%, 0.009%, or 0.01% EDTA, or any range thereof. In some embodiments, a pharmaceutical composition comprises 0.005% EDTA. In some aspects, the EDTA is at least, at most, exactly, or about pH 5.8 (e.g., pH 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, or 6.5, or any range thereof). In one embodiment, a pharmaceutical composition of the present disclosure comprises the following components: 50 mg/mL or 100 mg/mL of an anti-BMP9 antibody or antigen-binding fragment thereof (e.g., Ab1076) of the present disclosure, 20 mM histidine, 8.5% sucrose, and 0.02% polysorbate 80, 0.005% EDTA at pH 5.8. In one embodiment, a pharmaceutical composition comprises the following components: 100 mg/mL anti-BMP9 antibody or antigen-binding fragment thereof (e.g., Ab1076) or antigen-binding fragment of the present disclosure, 10 mM histidine, 5% sucrose, and 0.01% polysorbate 80 at pH 5.8. It is contemplated that any one or more of these foregoing ingredients may be excluded in an embodiment.

In some embodiments, a pharmaceutical composition comprises at least, at most, exactly, between (inclusive or exclusive) any two of, or about 0.2 mg/mL, 0.4 mg/mL, 0.6 mg/mL, 0.8 mg/mL, 1 mg/mL, 1.2 mg/mL, 1.4 mg/mL, 1.6 mg/mL, 1.8 mg/mL, or 2 mg/mL L-histidine, or any range thereof. In some embodiments, a pharmaceutical composition comprises 1.12 mg/mL L-histidine. In some embodiments, a pharmaceutical composition comprises at least, at most, exactly, between (inclusive or exclusive) any two of, or about 1.5 mg/mL, 1.7 mg/mL, 1.9 mg/mL, 2.1 mg/mL, 2.3 mg/mL, 2.5 mg/mL, 2.7 mg/mL, 2.9 mg/mL, 3.1 mg/mL, 3.3 mg/mL, or 3.5 mg/mL L-histidine hydrochloride monohydrate, or any range thereof. In some embodiments, a pharmaceutical composition comprises 2.67 mg/mL L-histidine hydrochloride monohydrate. In some embodiments, a pharmaceutical composition comprises at least, at most, exactly, between (inclusive or exclusive) any two of, or about 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, or 150 mg/mL sucrose, or any range thereof. In some embodiments, a pharmaceutical composition comprises 85 mg/mL sucrose. In some embodiments, a pharmaceutical composition comprises at least, at most, exactly, between (inclusive or exclusive) any two of, or about 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, or 0.1 mg/mL EDTA, or any range thereof. In some embodiments, a pharmaceutical composition comprises 0.05 mg/mL EDTA. In some aspects, the EDTA is at least, at most, exactly, or about pH 5.8 (e.g., pH 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, or 6.5, or any range thereof). In some embodiments, a pharmaceutical composition comprises at least, at most, exactly, between (inclusive or exclusive) any two of, or about 0.01 mg/mL, 0.05 mg/mL, 0.1 mg/mL, 0.15 mg/mL, 0.2 mg/mL, 0.25 mg/mL, 0.3 mg/mL, 0.35 mg/mL, or 0.4 mg/mL polysorbate 80, or any range thereof. In some embodiments, a pharmaceutical composition comprises 0.2 mg/mL polysorbate 80. In some aspects, the polysorbate 80 is at least, at most, exactly, or about pH 5.8 (e.g., pH 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, or 6.5, or any range thereof). In some embodiments, a pharmaceutical composition comprises an anti-BMP9 antibody, or antigen-binding fragment thereof, (e.g., Ab1076) at a concentration of 105 mg/mL (or about 99.6 mg/mL to about 118.2 mg/mL) or 100 mg/mL, 1.12 mg/mL L-histidine, 2.67 mg/mL L-histidine hydrochloride monohydrate, 85 mg/mL sucrose, 0.05 mg/mL edetate disodium dihydrate and 0.2 mg/mL polysorbate 80 at pH 5.8 in a nominal fill volume of 1.0 mL. Such pharmaceutical composition is suitable for SC or IV administration. It is contemplated that any one or more of these foregoing ingredients may be excluded in an embodiment.

### Therapeutic, Diagnostic, and Other Methods

The antibodies and the antibody conjugates of the present disclosure are useful in various applications including, but not limited to, therapeutic treatment methods and/or diagnostic treatment methods.

In some embodiments, antibodies or antigen-binding fragments thereof disclosed herein may inhibit, antagonize, and/or modulate the activity of BMP9 and its receptors and may be useful in the treatment, prevention, suppression, and/or amelioration of diseases, disorders, or conditions mediated by increased BMP9 activity, including but not limited to, hypertension (e.g., pulmonary arterial hypertension), anemias, liver diseases (e.g., liver fibrosis, cirrhosis, portal vein hypertension, end stage liver disease, such as varices, jaundice, ascites, hepatic encephalopathy, hepatorenal syndrome, spontaneous bacterial peritonitis, non-alcoholic fatty liver disease, portopulmonary hypertension, and hepato-pulmonary syndrome), neuroinflammatory or neurodegenerative diseases such as multiple sclerosis, fibrotic disorders, and/or heart failure. In some embodiments, the disease, disorder, or condition is selected from the group consisting of: hereditary hemorrhagic telangiectasia syndrome; cardiac valvular malformations; cardiac structural malformations; fibrodysplasia ossificans progressiva; juvenile familial polyposis syndrome; parathyroid disease; cancer (e.g., breast carcinoma, prostate carcinoma, renal cell carcinoma, bone metastasis, lung metastasis, osteosarcoma, and multiple myeloma); vascular calcification; vascular inflammation; atherosclerosis; acquired or congenital hypercholesterolemia or hyperlipoproteinemia; diseases, disorders, or syndromes associated with defects in lipid absorption or metabolism; diseases, disorders, or syndromes caused by hyperlipidemia; valve calcification; renal osteodystrophy; inflammatory disorders (e.g., ankylosing spondylitis); infections with viruses; bacteria; fungi; tuberculosis; and parasites.

In one aspect, the disclosure provides a method for treating hypertension, (e.g., pulmonary arterial hypertension). In some embodiments, the method of treating hypertension (e.g., pulmonary arterial hypertension) in a subject comprises administering to the subject in need thereof an effective amount of a pharmaceutical composition comprising any of the BMP9 antibodies or antigen-binding fragments thereof as described herein. In some embodiments, provided is a method of reducing or inhibiting BMP9 activity in a subject, comprising administering to the subject in need thereof an effective amount of a composition comprising an antibody or antigen-binding fragment thereof provided herein.

In another aspect, the disclosure further provides an antibody, an antigen-binding fragment thereof, or pharmaceutical composition thereof as described herein for use in the described method of treating hypertension (e.g., pulmonary arterial hypertension). The disclosure also provides for the use of an antibody, an antigen-binding fragment thereof, as described herein in the manufacture of a medicament for treating hypertension (e.g., pulmonary arterial hypertension).

In another aspect, provided is a method of one or more of detecting, prognosing, diagnosing, and/or monitoring diseases, disorders, or conditions mediated by increased and/or dysregulated BMP9 activity, including but not limited to, hypertension (e.g., pulmonary arterial hypertension), anemias, liver diseases (e.g., liver fibrosis, cirrhosis, portal vein hypertension, end stage liver disease, such as varices, jaundice, ascites, hepatic encephalopathy, hepatorenal syndrome, spontaneous bacterial peritonitis, non-alcoholic fatty liver disease, portopulmonary hypertension, and hepato-pulmonary syndrome), neuroinflammatory or neurodegenerative diseases such as multiple sclerosis, fibrotic disorders, and/or heart failure. In some embodiments, the methods comprise determining, measuring, detecting, or assaying the level of BMP9 in a sample obtained from a subject using the BMP9 antibodies, or an antigen-binding fragment thereof, described herein. For example, the anti-BMP9 antibodies, or an antigen-binding fragment thereof, as described herein can be labeled with a detectable moiety such as an imaging agent, an enzyme-substrate label, or other compounds or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Based on the determined, measured, detected, or assayed level of BMP9, different diagnoses and/or prognoses may be made for different subjects. The antibodies, or an antigen-binding fragment thereof, as described herein can also be used for in vivo diagnostic assays, such as in vivo imaging (e.g., PET or SPECT), or as a staining reagent.

In another aspect, provided is a method of monitoring the effectiveness of a therapy (e.g., BMP9 antibodies, or an antigen-binding fragment thereof, described herein) in a subject having a disease, disorder, or condition. The disease, disorder, or condition may be mediated by increased and/or dysregulated BMP9 activity, and may include, but is not limited to, hypertension (e.g., pulmonary arterial hypertension), anemias, liver diseases (e.g., liver fibrosis, cirrhosis, portal vein hypertension, end stage liver disease, such as varices, jaundice, ascites, hepatic encephalopathy, hepatorenal syndrome, spontaneous bacterial peritonitis, non-alcoholic fatty liver disease, portopulmonary hypertension, and hepato-pulmonary syndrome), neuroinflammatory or neurodegenerative diseases such as multiple sclerosis, fibrotic disorders, and/or heart failure. In specific aspects, the disease, disorder, or condition is pulmonary arterial hypertension. In some aspects, the method comprises administering a therapy (e.g., BMP9 antibodies, or an antigen-binding fragment thereof, described herein) to a subject in need thereof.

In some aspects, the method further comprises determining, measuring, detecting, or assaying the level of expression or activity of one or more biomarkers in a biological sample obtained from a subject. In some aspects, the one or more biomarkers include CXCL12, IGFBP4, INHBA, MALL, FRZB, CPE, CCL2, PDGFBB, Endothelin-1, or a combination thereof. Biomarker levels can be determined, measured, detected, or assayed in a biological sample obtained from a subject by measuring mRNA and/or protein levels. Protein levels may be determined, measured, detected, or assayed using, e.g., antibodies or antigen-binding fragments thereof that specifically bind the biomarkers. For example, the biomarker antibodies, or an antigen-binding fragments thereof, can be labeled with a detectable moiety such as an imaging agent, an enzyme-substrate label, or other compounds or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase.

In some aspects, the method further comprises comparing the level of the one or more biomarkers measured in the subject's biological sample to the level of the one or more biomarkers measured in a biological sample obtained from the subject before administration of the therapy (e.g., BMP9 antibodies, or an antigen-binding fragment thereof, described herein). In some aspects, the method further comprises determining the efficacy of the therapy (e.g., BMP9 antibodies, or an antigen-binding fragment thereof, described herein) based on the determined, measured, detected, or assayed level of the one or more biomarkers. Based on a profile of biomarker expression or activity levels, different treatments may be prescribed or recommended for different subjects and/or a treatment may be modified for a subject.

For example, in some aspects, the therapy (e.g., BMP9 antibodies, or an antigen-binding fragment thereof, described herein) is determined to be effective when the measured, detected, or assayed biomarker(s): i) are not measured, detected, or assayed in the biological sample from the subject; ii) are not significantly different than a control, wherein the control comprises a level of biomarker(s) that is representative of a level of biomarker(s) in a biological sample from a subject without the disease, disorder, or condition (e.g., PAH); iii) are less than a control, wherein the control comprises a level of biomarker(s) that is representative of a level of biomarker(s) in a biological sample from a subject with the disease, disorder, or condition (e.g., PAH); or iv) is decreased compared to a level of biomarker(s) before treatment of the subject with the therapy (e.g., BMP9 antibodies, or an antigen-binding fragment thereof, described herein). In some aspects, the therapy (e.g., BMP9 antibodies, or an antigen-binding fragment thereof, described herein) is determined to be ineffective when the biomarker(s): i) are measured, detected, or assayed in the biological sample from the subject; ii) are increased compared to a control, wherein the control comprises a level of biomarker(s) that is representative of a level of biomarker(s) in a biological sample from a subject without the disease, disorder, or condition (e.g., PAH); iii) is not significantly different or more than a control, wherein the control comprises a level of biomarker(s) that is representative of a level of biomarker(s) in a biological sample from a subject with the disease, disorder, or condition (e.g., PAH); or iv) is not significantly different or is increased compared to a level of biomarker(s) before treatment of the subject with the therapy (e.g., BMP9 antibodies, or an antigen-binding fragment thereof, described herein).

In some aspects, the method further comprises measuring, detecting, or assaying the level of biomarker(s) in a biological sample from the subject obtained prior to treatment. In some aspects, the method further comprises measuring, detecting, or assaying the level of biomarker(s) in a biological sample from the subject obtained after one or more treatments with the therapy e.g., BMP9 antibodies, or an antigen-binding fragment thereof, described herein). For example, the method may comprise or further comprise determining, measuring, detecting, or assaying biomarker levels after one dose, after two doses, after three doses, after four doses, after five doses, after six doses, or after more doses of the therapy (e.g., BMP9 antibodies, or an antigen-binding fragment thereof, described herein). Treatment efficacy may be determined based on the measured, detected, or assayed levels of the one or more biomarkers.

In some aspects, the determined, measured, detected, or assayed level of BMP9 and/or biomarkers is normalized. In some aspects, the level of BMP9 and/or biomarkers is compared to a control. In some aspects, the measured, detected, or assayed level of BMP9 and/or biomarkers is determined to be greater than the control. In some aspects, the measured, detected, or assayed level of BMP9 and/or biomarkers is determined to be less than the control. In some aspects, the subject has or has been determined to have a measured, detected, or assayed level of BMP9 and/or biomarkers in the sample that is greater than a level of BMP9 and/or biomarkers in a control sample. In some aspects, the subject has or has been determined to have a measured, detected, or assayed level of BMP9 and/or biomarkers in the sample that is less than a level of BMP9 and/or biomarkers in a control sample. In some aspects, the subject has or has been determined to have a measured, detected, or assayed level of BMP9 and/or biomarkers in the sample that is not significantly different than a level of BMP9 and/or biomarkers in a control sample. The control may comprise a level of BMP9 and/or biomarkers that is representative of a level of BMP9 and/or biomarkers in a sample from a subject with or without the disease, disorder, or condition (e.g., PAH). In some instances, the control may comprise a level of BMP9 and/or biomarkers that is representative of a level of BMP9 and/or biomarkers in a sample from a subject with the disease, disorder, or condition (e.g., PAH). In some aspects, the control may comprise a level of BMP9 and/or biomarkers that is representative of a level of BMP9 and/or biomarkers in a sample from a subject without the disease, disorder, or condition (e.g., PAH).

The subject samples for use in the methods described herein may be obtained using any method known to the art that can provide a sample suitable for the analytical methods described herein. The methods of obtaining a sample from a subject provided herein may include, for example, obtaining tissue, cells, or biological material by scraping, swabbing, biopsying, or otherwise collecting the tissue, cells, or biological material. A sample may include but is not limited to, tissue, cells, or biological material from cells or derived from cells of a subject. General methods for obtaining biological samples are also known in the art. For example, publications such as Ramzy, Ibrahim, Clinical Cytopathology and Aspiration Biopsy (2001), which is herein incorporated by reference in its entirety, describes general methods for biopsy and cytological methods. The biological sample may be a heterogeneous or homogeneous population of cells or tissues. In certain aspects, the sample is obtained from sources including, but not limited to, urine, blood, serum, plasma, sputum, mucus, saliva, exhaled breath condensate, bronchoalveolar lavage fluid, cerebrospinal fluid, sweat, tissue, hair follicle, buccal tissue, tears, menses, or feces. In specific aspects, the sample is obtained from serum. In certain aspects of the current methods, any medical professional such as a doctor, nurse or medical technician may obtain a biological sample for testing. Yet further, the biological sample can be obtained without the assistance of a medical professional. In some aspects of the present methods, the biological sample is obtained from a subject directly, from a medical professional, from a third party, or from a kit provided by a molecular profiling business or a third party. In some cases, the biological sample may be obtained after the subject, a medical professional, or a third party acquires and sends the biological sample to a third party. In some cases, the third party may provide suitable containers, and excipients for storage and transport of the biological sample to the third party.

Without wishing to be bound by theory, it is hypothesized that in diseased subjects (such as in patients with PAH), reduction in BMPRII either due to mutations or downregulation, shifts BMP9 from a vascular quiescence factor to a driver of vascular remodeling via modulation of EC proliferation and survival (i.e., BMPRII dysregulation leads to BMP9 pathogenic gain of function). As such, the methods comprise treating the subject with the BMP9 antibodies, antigen-binding fragments thereof, or pharmaceutical compositions thereof, described herein to block BMP9 binding to Type II receptors including BMPRII, ActRIIA, and ActRIIB, thereby neutralizing their downstream (pathological) signaling in endothelial cells (ECs) and smooth muscle cells (SMCs) dysregulated in PAH. In some embodiments, the BMP9 antibodies, antigen-binding fragments thereof, or pharmaceutical compositions thereof, described herein bind to BMP9 and block BMP9 binding to Type II receptors by at least, at most, exactly, between (inclusive or exclusive) any two of, or about 5%, 10%, 15%, 20%, 25%, 30%, 35%, 40%, 45%, 50%, 55%, 60%, 65%, 70%, 75%, 80%, 85%, 90%, 95%, or 100%, or any range thereof (e.g., by at least 5%, at least 10%, at least 15%, at least 20%, at least 30%, at least 40%, at least 50%, at least 60%, at least 70%, at least 80%, at least 90%, or at least 100%), as compared to before administration.

With respect to all methods described herein, reference to anti-BMP9 antibodies also includes antigen-binding fragments thereof and pharmaceutical compositions comprising the anti-BMP9 antibodies or antigen-binding fragments thereof and one or more additional agents.

### Administration and Dosing

Typically, an antibody, or antigen binding fragment thereof, of the disclosure is administered in an amount effective to treat a condition as described herein. The antibodies of the disclosure can be administered as an antibody per se, or alternatively, as a pharmaceutical composition containing the antibody.

In certain embodiments, the antibodies, or antigen binding fragments thereof, or pharmaceutical compositions thereof, of the present disclosure are administered by any suitable route in the form of a pharmaceutical composition adapted to such a route and in a dose effective for the treatment intended.

In some embodiments, the antibodies, or antigen binding fragments thereof, or pharmaceutical compositions thereof, may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including microneedle) injectors, needle-free injectors, and infusion techniques. In some embodiments, the antibodies, or antigen binding fragments thereof, or pharmaceutical compositions thereof, may be administered subcutaneously. In some embodiments, the antibodies, or antigen binding fragments thereof, or pharmaceutical compositions thereof, may be administered intravenously. It is contemplated that any one or more of these foregoing routes of administration may be excluded in an embodiment.

In another embodiment, the compounds disclosed herein may also be administered topically to the skin or mucosa, that is, dermally, transdermally, or sublingually. In another embodiment, the compounds disclosed herein can also be administered intranasally or by inhalation. In another embodiment, the compounds disclosed herein may be administered rectally or vaginally. In another embodiment, the compounds disclosed herein may also be administered directly to the eye or ear. It is contemplated that any one or more of these foregoing routes of administration may be excluded in an embodiment.

The dosage regimen for the antibodies, or antigen binding fragments thereof, of the present disclosure, or compositions containing said antibodies, is based on a variety of factors, including the type, age, weight, sex, and/or medical condition of the subject; the severity of the condition; the route of administration; and the activity of the particular antibody employed. Thus, the dosage regimen may vary widely. In one embodiment, the total daily dose of an antibody of the disclosure is typically from about 0.01 to about 1000 mg/kg (i.e., mg antibody of the disclosure per kg body weight) (e.g., at least, at most, exactly, between (inclusive or exclusive) any two of, or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1000 mg/kg, or any range thereof), for the treatment of the indicated conditions discussed herein. In another embodiment, total daily dose of the antibody of the disclosure is from about 0.1 to about 50 mg/kg, and in another embodiment, from about 0.5 to about 30 mg/kg. In some embodiments, the daily dose is about 0.5 mg/kg (i.e., about 35 mg/daily in a typical sized human).

In some embodiments, the method or use comprises administering a dose of about 1 mg to about 1000 mg. In some embodiments, the method or use comprises administering a dose of about 1 mg to 1000 mg (e.g., at least, at most, exactly, between (inclusive or exclusive) any two of, or about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1000 mg/kg, or any range thereof) as an initial fixed dose. In some embodiments, the method or use comprises administering a dose of about 1 mg to about 2 mg, about 2 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 150 mg, about 150 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, or about 900 mg to about 1000 mg, optionally, as an initial fixed dose. In some embodiments, the method or use comprises administering a dose of about 15 mg, 30 mg, 40 mg, 50 mg, 75 mg, 100 mg, 150 mg, 300 mg, 500 mg, 600 mg, 700 mg, 800 mg, 900 mg, or 1000 mg of an anti-BMP9 antibody or a pharmaceutical composition thereof. In some embodiments, the dose is an initial fixed dose.

In some embodiments, the method or use comprises administering a dose of about 12 mg to 500 mg (e.g., at least, at most, exactly, between (inclusive or exclusive) any two of, or about 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, or 500 mg/kg, or any range thereof) subcutaneously every 2 weeks every 3 weeks or every 4 weeks. In some embodiments, the method of use comprises subcutaneously administering a dose of about 12 mg, about 15 mg, about 20 mg, about 24 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 210 mg, about 220 mg, about 230 mg, about 240 mg, about 250 mg, about 300 mg, about 350 mg, about 400 mg, about 450 mg. or about 500 mg every 2 weeks, every 3 weeks or every 4 weeks. In some embodiments, the method or use comprises administering a dose of about 110 mg subcutaneously every 3 weeks. In some embodiments, the method or use comprises administering a dose of about 30 mg, about 50 mg about 75 mg, about 100 mg, about 150 mg, about 165 mg, about 180 mg, or about 200 mg every 2 weeks. In some the method or use comprises administering a dose of about 165 mg every 2 weeks. In some embodiments, the method or use comprises administering a dose of about 50 mg, about 120 mg, about 230 mg, about 240 mg, about 250 mg, about 255 mg, about 260 mg, about 270 mg, about 280 mg, about 290 mg, or about 300 mg every 3 weeks. In some aspects, the method or use comprises administering a dose of about 255 mg every 3 weeks. In some embodiments, the method or use comprises administering a dose of about 65 mg, about 160 mg, about 200 mg, about 250 mg, about 300 mg, about 325 mg, about 350 mg, about 360 mg, about 370 mg, about 380 mg about 390 mg, about 400 mg every 4 weeks. In some aspects, the method or use comprises administering a dose of about 355 mg every 4 weeks. In some embodiments, the antibody is Ab1076.

The dose administered provides an average suppression of about 90% to about 99% (e.g., at least, at most, exactly, between (inclusive or exclusive) any two of, or about 80%, 81%, 82%, 83%, 84%, 85%, 86%, 87%, 88%, 89%, 90%, 91%, 92%, 93%, 94%, 95%, 96%, 97%, 98%, 99%, or 100%, or any range thereof) of free BMP9 at steady state in a standard antagonistic antibody model. In some embodiments, the antibody, or antigen binding fragment thereof, or a pharmaceutical composition thereof, is administered subcutaneously at about 30 mg, 35 mg, about 50 mg, about 75 mg, about 85 mg, about 100 mg, about 125 mg, about 150 mg, or about 165 mg every 2 weeks and provides a 95%, 98%, or 99% average suppression of free BMP9 at steady state. In some embodiments, the antibody, or antigen binding fragment thereof, or a pharmaceutical composition thereof, is administered subcutaneously at about 50 mg, about 55 mg, about 120 mg, about 130 mg, about 150 mg, about 200 mg, about 230 mg, about 250 mg, or about 255 mg every 3 weeks and provides a 95%, 98%, or 99% average suppression of free BMP9 at steady state. In some embodiments, the antibody, or antigen binding fragment thereof, or a pharmaceutical composition thereof, is administered subcutaneously at about 65 mg, about 75 mg, about 100 mg, about 160 mg, about 180 mg, about 200 mg, about 250 mg, about 300 mg, about 325 mg, about 350 mg, or about 355 mg every 4 weeks and provides a 95%, 98%, or 99% average suppression of free BMP9 at steady state. In some embodiments, the antibody is Ab1076.

In some embodiments, the method or use comprises administering a dose of about 2 mg, 10 mg, 20 mg, 30 mg, 40 mg, 50 mg, 75 mg, 100 mg, 110 mg, 125 mg, 150 mg, 200 mg, 250 mg, 300 mg, 350 mg, 400 mg, 450 mg, or 500 mg of an anti-BMP9 antibody, or an antigen-binding fragment thereof, or a pharmaceutical composition thereof of the disclosure. In some embodiments, the method or use comprises administering a dose of about 2 mg, 10 mg, 30 mg, 50 mg, 75 mg, 100 mg, 125 mg, 150 mg, 200 mg, 250 mg, 300 mg, 350 mg, 400 mg, 450 mg, or 500 mg of an anti-BMP9 antibody, or an antigen-binding fragment thereof, or a pharmaceutical composition thereof of the disclosure on a weekly basis. In some embodiments, the method or use comprises administering a dose of about 2 mg, 10 mg, 20 mg, 30 mg, 40 mg, 50 mg, 75 mg, 100 mg, 125 mg, 150 mg, 200 mg, 250 mg, 300 mg, 350 mg, 400 mg, 450 mg, or 500 mg of an anti-BMP9 antibody (e.g., Ab1076), or antigen binding fragment thereof, or a pharmaceutical composition thereof, subcutaneously, on a weekly basis. In some embodiments, the method or use comprises administering a dose of about 2 mg, 10 mg, 20 mg, 30 mg, 40 mg, 50 mg, 75 mg, 100 mg, 125 mg, 150 mg, 200 mg, 250 mg, 300 mg, 350 mg, 400 mg, 450 mg, or 500 mg of an anti-BMP9 antibody (e.g., Ab1076), or antigen binding fragment thereof, or a pharmaceutical composition thereof, subcutaneously, on a monthly basis. The required dose may be given subcutaneously as a single injection or may be delivered in more than one injection in one or more injection sites. For example, the required dose may be administered subcutaneously as two injection shots once a month.

In some embodiments, the method or use comprises administering dose of about 0.01 mg/kg to about 300 mg/kg, about 1 mg/kg to about 250 mg/kg, about 10 mg/kg to about 200 mg/kg, about 50 mg/kg to about 150 mg/kg, or about 75 mg/kg to about 100 mg/kg of an antibody, or antigen binding fragment thereof, or a pharmaceutical composition, optionally as an initial dose. The initial dose may be followed by one or more subsequent doses. In some embodiments, one or more subsequent dose may be administered at least any of weekly, every other week, every three weeks, every four weeks, every five weeks, every six weeks, every seven weeks, every eight weeks, every nine weeks, every ten weeks, every eleven weeks, or every twelve weeks. In some embodiments, the dose is administered every three weeks.

An initial dose may be followed by one or more subsequent doses. In some embodiments, a subsequent dose is the same dose, a lower dose, or a higher dose of anti-BMP9 antibody as compared to the initial dose. In some embodiments, one or more subsequent dose may be administered at least any of weekly, every other week, every three weeks, every four weeks, every five weeks, every six weeks, every seven weeks, every eight weeks, every nine weeks, every ten weeks, every eleven weeks, or every twelve weeks. A specific dose protocol is one involving the maximal dose or dose frequency that avoids significant undesirable side effects.

It is contemplated that any one or more of these foregoing doses and/or dosing regimens may be excluded in an embodiment.

### Co-Administration

The antibodies or antigen-binding fragments thereof of the disclosure can be used alone, or in combination with one or more other therapeutic agents. The present disclosure provides any of the uses, methods or compositions as defined herein wherein an antibody or antigen-binding fragment thereof of the disclosure is used in combination with one or more other therapeutic agent. For example, in the treatment of pulmonary arterial hypertension, the BMP9 antibodies or antigen-binding fragments thereof described herein may be used in combination with one or more of current pulmonary arterial hypertension therapies. Examples of such therapies include but are not limited to, vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of pulmonary hypertension may also be carried out in conjunction with oxygen therapy, atrial septostomy, pulmonary thromboendarterectomy, and/or lung and/or heart transplantation. It is contemplated that any one or more of these foregoing therapies may be excluded in an embodiment.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

### Kits

Another aspect of the disclosure provides kits comprising the antibody or antigen-binding fragment thereof of the disclosure (e.g., Ab1076) or pharmaceutical compositions comprising the antibody or antigen-binding fragment thereof (e.g., Ab1076). A kit may include, in addition to the antibody or antigen-binding fragment thereof or pharmaceutical composition thereof of the disclosure, diagnostic or therapeutic agents. A kit may also include instructions for use in a diagnostic or therapeutic method. In some embodiments, the kit includes the antibody or antigen-binding fragment thereof or a pharmaceutical composition thereof and a diagnostic agent.

In other embodiments, the kit includes the antibody or antigen-binding fragment thereof or a pharmaceutical composition thereof and one or more therapeutic agents, such as prostacyclin, epoprostenol, and sildenafil; endothelin receptor antagonists such as bosentan; calcium channel blockers such as amlodipine, diltiazem, and nifedipine; anticoagulants such as warfarin; and/or diuretics. It is contemplated that any one or more of these foregoing therapeutic agents may be excluded in an embodiment.

In yet another embodiment, the disclosure comprises kits that are suitable for use in performing the methods of treatment described herein. In one embodiment, the kit contains a first dosage form comprising one or more of the antibodies or antigen-binding fragments thereof of the present disclosure in quantities sufficient to carry out the methods disclosed herein. In another embodiment, the kit comprises one or more antibodies or antigen-binding fragments thereof of the present disclosure in quantities sufficient to carry out the methods disclosed herein and at least a first container for a first dosage and a second container for a second dosage.

### Biological Deposit

Representative materials of the present disclosure were deposited in the American Type Culture Collection, 10801 University Boulevard, Manassas, VA 20110-2209, USA, on Apr. 19, 2022. Vector “Ab1076-VH” having ATCC Accession No. PTA-127292 comprises a DNA insert encoding the anti-BMP9 heavy chain variable region and vector “Ab1076-VL” having ATCC Accession No. PTA-127293 comprises a DNA insert encoding the anti-BMP9 light chain variable region.

| | | SEQ | ATCC |
|---|---|---|---|
| Antibody | Description | ID NO: | Accession No. |
| Ab1076 | Ab1076-VH (heavy chain variable | 77 | PTA-127292 |
| | region of antibody Ab1076) | | |
| Ab1076 | Ab1076-VL (light chain variable | 74 | PTA-127293 |
| | region of antibody Ab1076) | | |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

### EXAMPLES

Below are examples of specific aspects for carrying out the present disclosure. The following examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, those of skill in the art should, in light of the present disclosure, appreciate that many changes may be made in the specific aspects which are disclosed and still obtain a like or similar result without departing from the spirit and scope of the disclosure. Efforts have been made to ensure accuracy with respect to numbers used (e.g., amounts, temperatures, etc.), but some experimental error and deviation should, of course, be allowed for.

### Example 1: Selection of Anti-BMP9 Antibodies by Phage Display

Anti-BMP9 single-chain Fv (scFv) hits were selected from the human phage display antibody library WyHN5 by panning on the mature human BMP9 as follows.

Human mature BMP9 protein was biotinylated with sulfo-NHS-LC-Biotin (Thermo) according to the manufacturer's protocol. Four rounds of selection were performed in solution with decreasing concentrations of biotinylated human BMP9, as follows: 200 nM biotinylated BMP9 (1^{st} round), 50 nM biotinylated BMP9 (2^{nd} round), 1 nM biotinylated BMP9, then add 1 μM non-biotinylated BMP9 and incubate overnight (3^{rd} round), and 1 nM biotinylated BMP9 (4^{th} round). In each round, the phage bonded to biotinylated BMP9 were captured with magnetic DYNABEADS® M-280 Streptavidin (Invitrogen) followed by washing to remove non-specifically bound phage. BMP9-specific binders were eluted with 100 mM triethylamine, followed by amplifying in E. coli for next rounds of selection and/or screening ELISA.

ScFv can be expressed either on the surface of a phage particle or in solution in the bacterial periplasmic space, depending upon the growth conditions used. To induce release of scFv into the periplasm, 96-well plates containing 2×YT media with 0.1% glucose and 100 μg/ml ampicillin were inoculated from 2×YT agar plates (one clone per well) and grown at 37° C. with shaking at 850 rpm for −4 hrs. Cultures were induced with IPTG at a final concentration of 0.02 mM and grown overnight at 30° C. with shaking at 850 rpm. The contents of the bacterial periplasm (peripreps) were released by osmotic shock. Briefly, plates were centrifuged, and pellets were resuspended in 150 ml TES periplasmic buffer (50 mM Tris, 1 mM EDTA, 20% Sucrose, pH 7.4), followed by the addition of 150 μl 1:5 TES:water, and incubated on ice for 30 min. Plates were centrifuged for 20 min at 4000 rpm, and the scFv-containing supernatants were harvested.

For binding assays with scFv peripreps, high-binding 96-well ELISA plates (Costar 3590) were coated with 0.1 μg per well (100 μl of 1 μg/ml) of human BMP9 in PBS buffer overnight at 4° C. After discarding coating solution, plates were blocked at room temperature for 2 hrs with PBS+3% BSA. After discarding blocking solution, 100 μl of peripreps was added. After 1 hr incubation at room temperature with slow shaking, plates were washed 5 times with 300 μl/well of washing buffer (Perkin Elmer 1244-114) and incubated for 1 hr with 100 μl of secondary HRP-labeled anti-His tag Ab. After washing 5× with 300 μl per well of washing buffer, plates were developed with TMB substrate solution for 10 min, then 0.18 M H₂SO₄ was added to stop the reaction. Absorbance at 450 nM was measured, and data were plotted and analyzed with Microsoft Excel. From this screen, 100 scFv hits were identified as binding to human BMP9 by periprep ELISA, and 15 unique clones, including lead clones BMP9-0093 (also referred to as Ab93 herein) and BMP9-0101 (also referred to as Ab101 herein), were subsequently converted to human IgG₁/lambda using standard molecular biology methods.

### Example 2: Binding of Anti-BMP9 Antibodies to Human BMP9 Protein

This example illustrates the binding of the identified anti-BMP9 antibodies to human BMP9 protein in an enzyme-linked immunosorbent assay (ELISA).

Purified human mature BMP9 protein was coated on a NUNC™-MAXISORP™ 96-well ELISA plates at 1 μg/ml in 100 μl of PBS (calcium and magnesium free) at 4° C. The plates were washed 3 times with PBS+0.05% TWEEN™ 20 and blocked with PBS+1% BSA for 1 hr at RT with shaking. Tested Abs were serially diluted 5-fold in PBS+1% BSA, starting from 100 nM. Blocking solution was removed, and plates were washed as before, followed by adding serially diluted Abs and incubating for 1 hr at RT with shaking. Plates were washed as before, and a secondary antibody (goat anti-human IgG Fc-HRP from Invitrogen, cat #31413, 1:10000) was added, followed by 1 hr incubation at RT. Plates were washed as before, signal was developed using TMB substrate, and the reaction was stopped with 0.18 M H₂SO₄. Absorbance was read at 450 nm on an ENVISION® plate reader (Perkin Elmer).

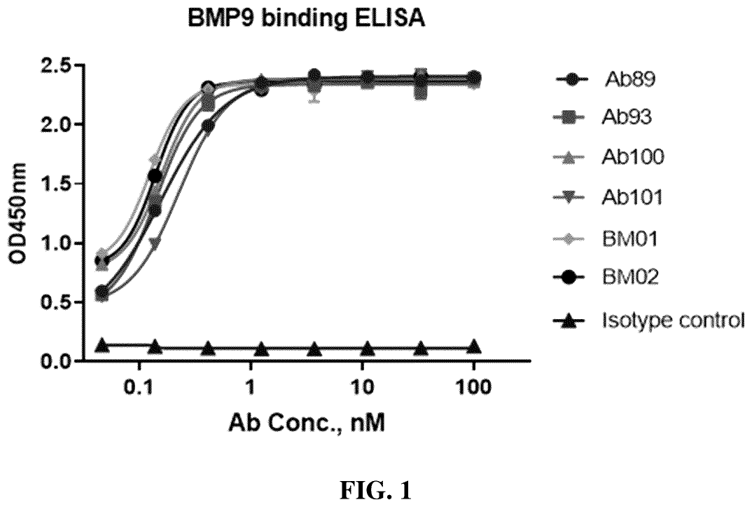

The result is shown in FIG. 1. All identified antibodies (Ab89, Ab93, Ab100 and Ab101) bound human mature BMP9 with high potency. BM01 (referred to as AM0100 in WO 2016/193872) and BM02 (referred to as AM4405 in WO 2016/193872) were used as reference antibodies. In contrast, the isotype control antibody failed to bind human BMP9.

### Example 3: Competition ELISA to Measure Neutralization Activity of Anti-BMP9 Antibodies

This example illustrates the ability of the identified anti-BMP9 antibodies to inhibit the interactions between BMP9 and its receptors in a blocking (competition) ELISA.

Type I receptor (ALK1) and type II receptors (BMPRII, ActRIIA, or ActRIIB) were used to see if anti-BMP9 Abs can compete with binding of these receptors to BMP9. Each receptor was coated on an ELISA plate at 1-15 μg/ml and then incubated with serially diluted biotinylated BMP9 in order to determine optimal receptor coating concentration and determine the EC₉₀ of biotinylated BMP9 binding to each receptor (data not shown).

For competition ELISAs, each receptor protein was coated on a 384-well ELISA plate at its optimal coating concentration, 20 μl/well in PBS-CMF at 4° C. overnight. The next day, plates were washed 3 times with PBS+0.05% TWEEN™ 20 and blocked with PBS+1% BSA for 1 hr at RT with shaking. Anti-BMP9 Abs were serially diluted 3-fold in PBS+1% BSA, starting from 100 nM, then mixed with the same volume of the optimal concentration of biotinylated BMP9. The final concentration of biotinylated BMP9 corresponded to the EC₉₀ for each receptor, as determined in a preliminary experiment described above.

Serially diluted Abs were incubated with biotinylated BMP9 for at least 15 min at RT. Plates were washed as before, followed by addition of serially diluted Abs and the biotinylated BMP9 mixture. After incubating for 1 hr at RT, plates were washed 3 times with PBS+0.05% TWEEN™ 20. Secondary antibody was added (HRP-Streptavidin from BioLegend, cat #405210, 1:2000), followed by 1 hr incubation at RT. Plates were washed as before, signal was developed using TMB substrate, and the reaction stopped with 0.18 M H₂SO₄. Absorbance was read at 450 nm on an ENVISION® plate reader (Perkin Elmer).

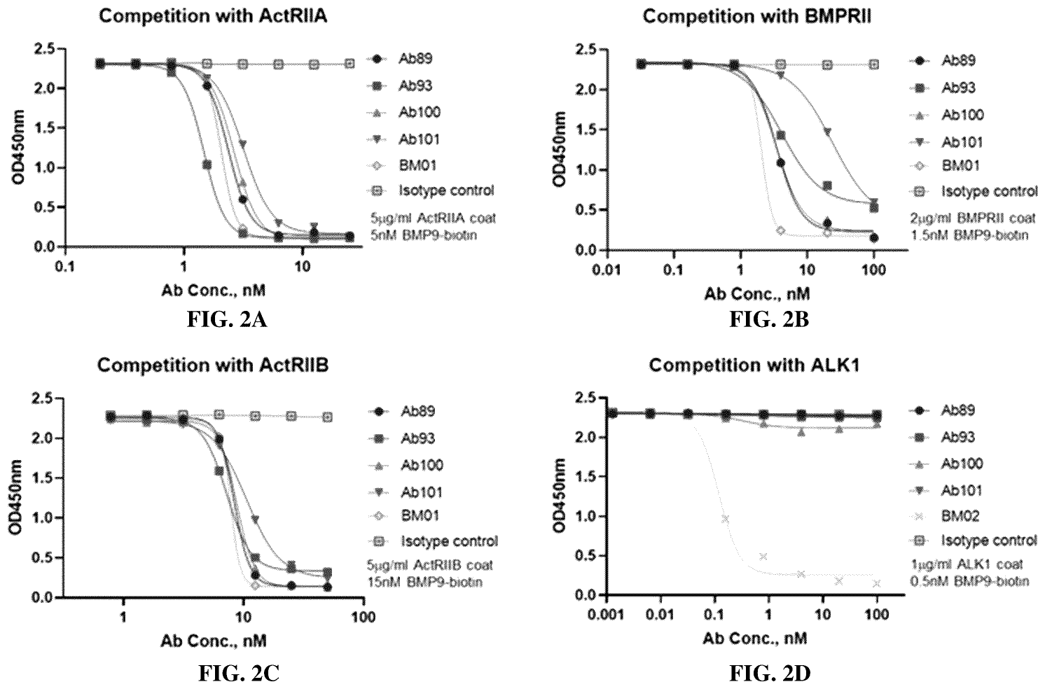

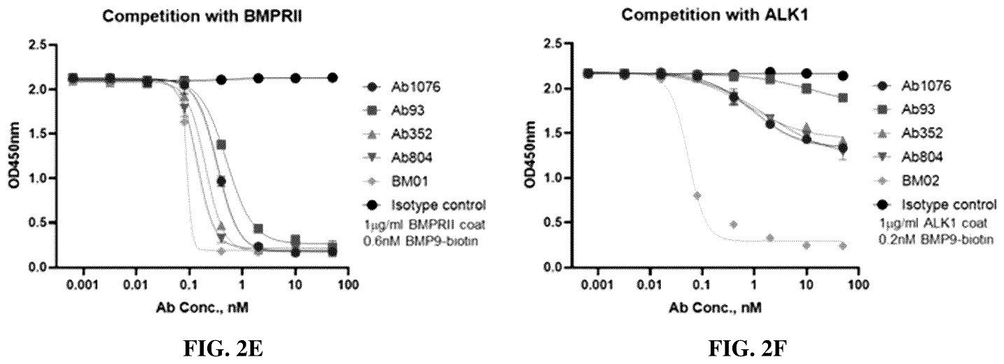

Results of these competition ELISAs are shown in FIG. 2. Antibodies Ab89, Ab93, Ab100, and Ab101 inhibit binding of BMP9 to type II receptors (BMPRII, ActRIIA, or ActRIIB), but not binding of BMP9 to type I receptor ALK1 (FIGS. 2A-2D). Antibodies Ab352, Ab804, and Ab1076, derived from parental antibody Ab93, strongly inhibit binding of BMP9 to type II receptor (BMPRII) and weakly inhibit binding of BMP9 to type I receptor ALK1 (FIGS. 2E-2F). In contrast, reference antibody BM02 is a type I receptor blocker, as it strongly inhibited binding of BMP9 to ALK1 (FIG. 2F) and did not inhibit type II receptor binding (not shown).

### Example 4: Effect of Anti-BMP9 Antibodies on BMP9 Binding to Endothelial Cells

This example illustrates the ability of the identified anti-BMP9 antibodies to inhibit binding of human BMP9 to endothelial cells in a dose-dependent manner.

Human telomerase-immortalized microvascular endothelial (TIME) cells were obtained from ATCC and cultured according to the provider's instructions. Recombinant human BMP9 was conjugated to NHS-ALEXA FLUOR 647 ® dye. On the day of experiment, the cells were collected by enzymatic detachment and resuspended in pre-chilled growth medium. Identical aliquots of the cell suspension were then dispensed into wells of 96-well plates and placed on ice.

BMP9 Abs were serially diluted in pre-chilled cell growth medium to 4× of the final concentrations of 0.2 nM, 1 nM, 5 nM, 25 nM, and 125 nM. BMP9-ALEXA FLUOR 647 ® protein was diluted in pre-chilled cell growth medium to 4× of the final concentration of 1 nM, to be combined with the serially-diluted BMP9 Abs at a 1:1 (v/v) ratio, resulting in a 2× of the final concentrations of BMP9 and Abs, respectively. Additionally, an aliquot of the 4× BMP9 solution was combined 1:1 (v/v) with pre-chilled medium without the Abs.

The above samples were then incubated with refrigeration and protected from light for 40 min, prior to dispensing aliquots from each sample at the 1:1 (v/v) ratio to the wells containing the TIME cell suspensions in 96-well plates. Cell samples mixed with medium without BMP9 or Abs were considered untreated control. The plates were further incubated for 60 min on ice protected from light, after which the samples in wells were washed with excess of pre-chilled buffer containing fetal bovine serum protein and sodium azide to prevent active uptake of materials from the cell environment.

The samples were then analyzed with BD FACSYMPHONY™ A5 flow cytometer equipped with an appropriate optical setup for the analysis of ALEXA FLUOR 647 ® dye median intensity per cell using BD FACSDIVA™ software. The raw data files were exported for the analysis with FlowJo software, and the numerical data was plotted using GraphPad Prism.

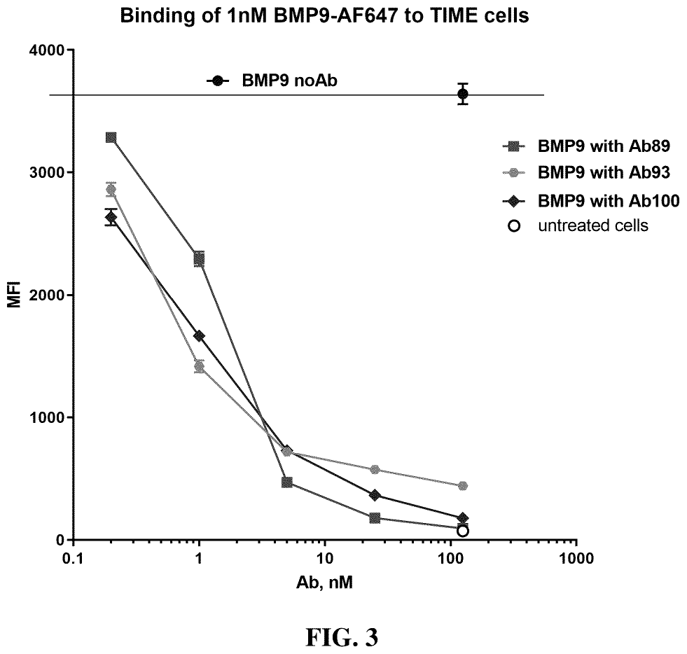

The result is shown in FIG. 3. Anti-BMP9 antibodies Ab89, Ab93, and Ab100 demonstrated a dose-dependent inhibition of BMP9 binding to TIME cells.

### Example 5: Determination of Affinity of Ab1076 to Human, Cyno and Rat BMP9

This example confirms the binding affinity of anti-BMP9 antibody Ab1076 (analyzed as a Fab) to human, cyno. and rat BMP9.

The binding affinities of Ab1076 (as a monovalent Fab fragment) to mature human, cyno, and rat BMP9 were determined by surface plasmon resonance (SPR) using a BIACORE™ T200 (Cytiva) at 37° C. with a collection rate of 10 Hz. The running and sample buffer was 10 mM HEPES pH 7.4, 0.15 M NaCl, 3 mM EDTA, 0.05% P-20 (HBS-EP+). Biotinylated mature human, cyno, and rat BMP9 proteins were reversibly captured onto a Sensor Chip CAP using the Biotin CAPture Reagent (Cytiva, 28920234) according to the manufacturer's instructions. The final capture level of human BMP9 was 100 resonance units (RU), cyno BMP9 was 50 RU, and rat BMP9 was 60 RU. Flow cell 1, without BMP9, was used as a reference flow cell. A two-fold dilution series of the Ab1076 Fab ranging from 100 nM to 6.2 nM was injected for 60 seconds at a flow rate of 100 μl per minute and the dissociation was monitored for 1800 seconds. Rate constants and affinities were determined by fitting the double referenced sensorgram data to a 1:1 model in BIACORE™ Insight Evaluation software version 3.0.1.12.15655 (Cytiva).

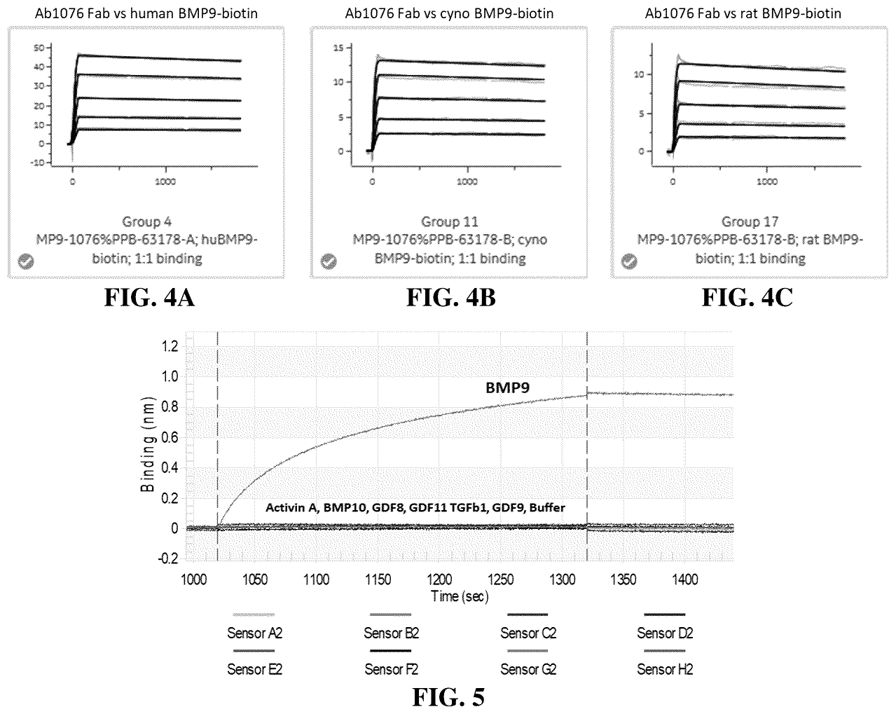

As summarized in Table 1, the equilibrium dissociation constant (K D) values for human, cyno, and rat BMP9 are 87.41 pM, 65.82 pM and 98.63 pM respectively. Cyno and rat BMP9 K_{D} values are within 2-fold of human BMP9. Representative sensorgrams are shown in FIG. 4.

TABLE 1
Affinities of Ab1076 Fab fragment to human, cyno, and rat
BMP9 determined by SPR at 37° C. Sensorgram data of 3
parallel measurements from 3 independent dilutions of Ab1076
Fab analyte were used to calculate kₐ, k_{d} and K_{D} ± SD values.

| | | | | K_{D} |
|---|---|---|---|---|
| Analyte | Ligand | kₐ (1/Ms) | k_{d} (1/s) | (pM), +/− SD |
| Ab1076 | Human BMP9- | 4.40E+05 | 3.84E−05 | 87.41 ± 6.99 |
| (Fab) | biotin | | | |
| | Cyno BMP9- | 5.85E+05 | 3.84E−05 | 65.82 ± 6.48 |
| | biotin | | | |
| | Rat BMP9- | 5.32E+05 | 5.14E−05 | 98.63 ± 19.2 |
| | biotin | | | |

### Example 6: Evaluation of Ab1076 for Off-Target Binding to a Panel of TGF13 Family Members

In this example, the off-target binding of Ab1076 (tested as a Fab) to a panel of TGFβ family members was evaluated.

Bio-Layer Interferometry (BLI) with an OCTET® Red 384 instrument was used to evaluate Ab1076 Fab for off-target binding to a panel of TGFβ family members including human TGFβ1, GDF8, GDF9, GDF11, Activin A, and BMP10 at 37° C. Human BMP9 was used as a positive control. The TGF13 family members were amine coupled to AR2G biosensors (Sartorius ForteBio, 18-5092) at 20 μg/ml for 300 seconds. Immobilization levels were as follows: GDF8, 0.72 nm; GDF11, 0.28 nm; BMP9, 0.58 nm; BMP10, 1.0 nm; GDF9, 0.35 nm; TGFβ1, 0.62 nm and Activin A, 0.6 nm. The biosensors with immobilized TGFβ family members were dipped into Ab1076 Fab diluted to 100 nM in Kinetics buffer (Sartorius ForteBio 18-5032) for 300 seconds. The dissociation was followed for 180 seconds. Data was collected using OCTET® Acquisition software version 11.0.0.64 and double referenced in OCTET® Data Analysis software version 11.0.

At 100 nM, Ab1076 Fab demonstrated no observable off-target binding to the evaluated TGFβ family members but did bind BMP9 as expected (FIG. 5). All of the TGFβ family members were active as demonstrated by their binding to their respective positive controls (not shown).

### Example 7: Anti-BMP9 Antibodies Inhibit BMP9-Induced Phospho-SMAD1/5/9 and Phospho-SMAD2 Signaling in Endothelial Cells

In this example, the ability of anti-BMP9 antibodies to inhibit BMP9-induced phospho-SMAD1/5/9 and phospho-SMAD2 nuclear translocation as a measure of activation of SMAD1/5/9 or SMAD2 signaling in human, cynomolgus monkey, and rat endothelial cells was tested. Briefly, TIME cells (immortalized human microvascular endothelial cell line), primary human pulmonary artery endothelial cells (HPAEC), primary cynomolgus monkey pulmonary artery endothelial cells (PAEC), and primary rat liver sinusoidal endothelial cells (LSEC) were treated with 10 pM or 1000 pM of human or species-specific BMP9 in the presence of anti-BMP9 antibodies for 30 minutes. The cells were fixed, blocked and permeabilized, and stained overnight with an antibody against pSMAD1/5/9 or pSMAD2. The next day, the cells were washed and stained with a secondary antibody labeled with ALEXA FLUOR® 488 to detect pSMAD1/5/9 or pSMAD2, Hoechst 33342 to detect nuclei, and a cell mask deep red stain to delineate whole cells. After a final wash, the cells were imaged on a high content imager. To analyze pSMAD1/5/9 or pSMAD2 nuclear translocation, the average nuclear fluorescence in the ALEXA FLUOR® 488 channel was calculated for each well of cells. IC₅₀ values were calculated using GraphPad Prism Version 9.0.0.

Dose-dependent inhibition of BMP9-induced pSMAD1/5/9 and pSMAD2 nuclear translocation was observed in all endothelial cells upon treatment with anti-BMP9 antibodies.

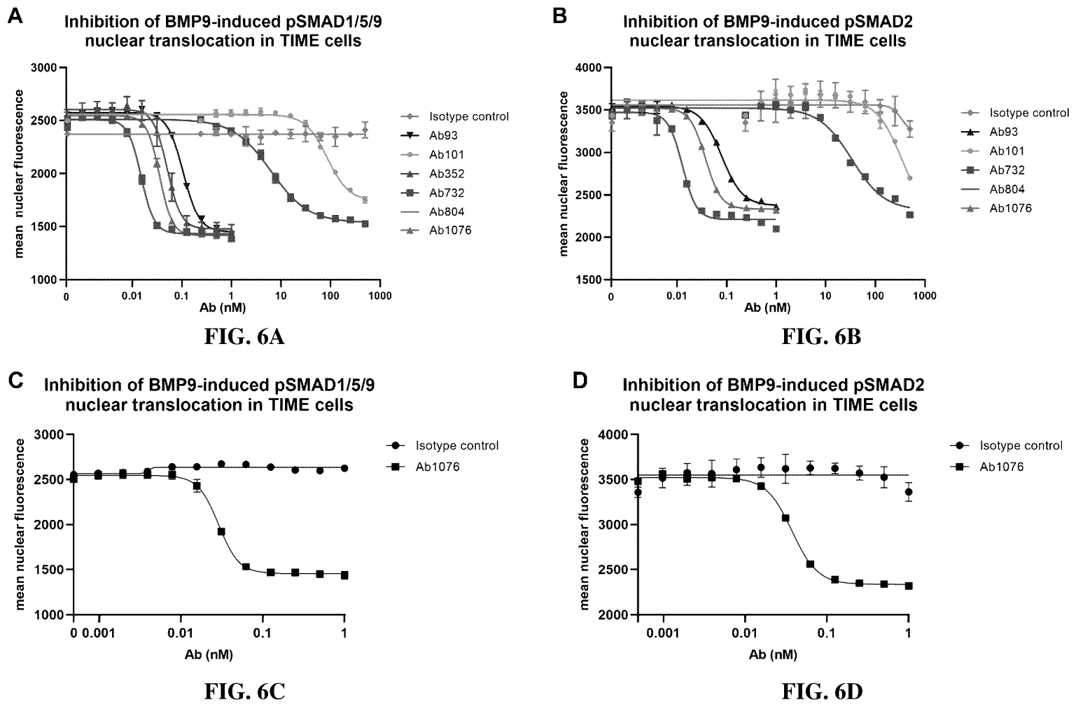

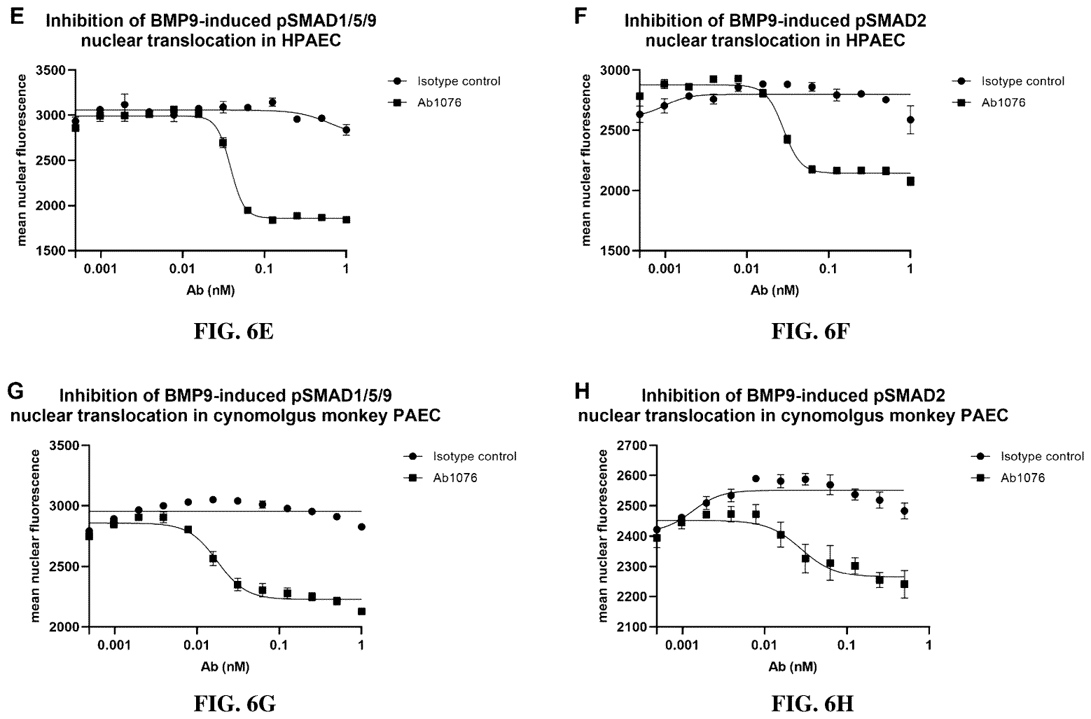

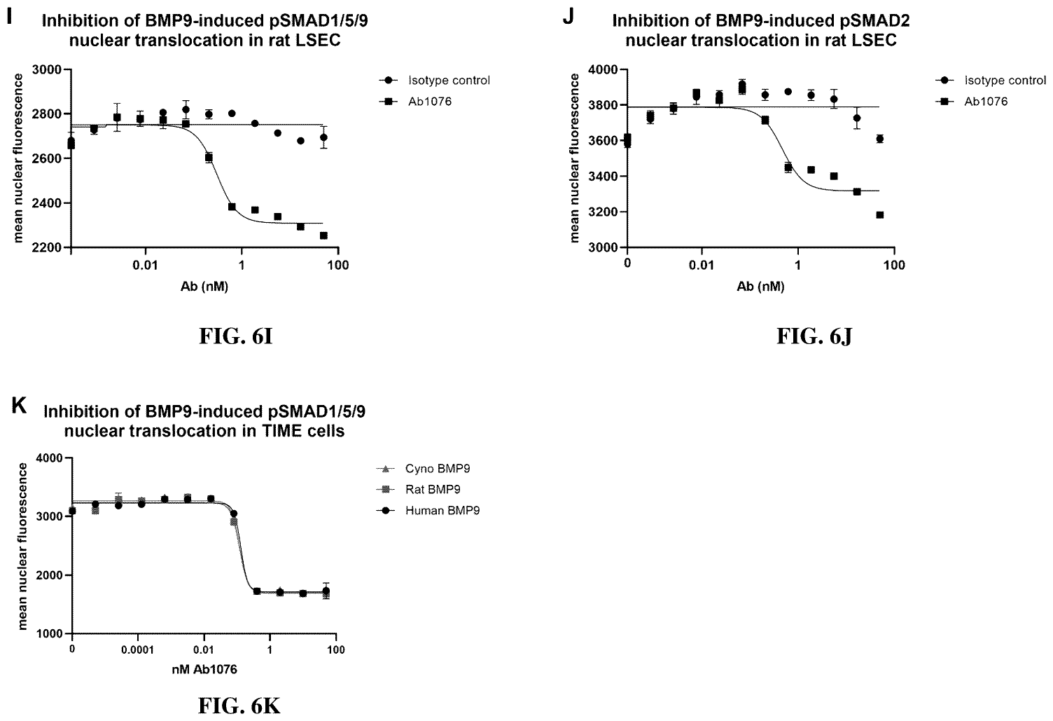

In several experiments performed, the IC₅₀ values of anti-BMP9 antibodies for human BMP9 in the pSMAD1/5/9 assay ranged from 0.02 nM-86 nM. The IC₅₀ values of anti-BMP9 antibodies for cynomolgus monkey BMP9 ranged from 17 pM-117 pM and from 120 pM-300 pM for rat BMP9 in the pSMAD1/5/9 assay. The IC₅₀ value of anti-BMP9 antibodies for human BMP9 in the pSMAD2 assay ranged from 0.01 nM-330 nM in several experiments performed. The IC₅₀ values of Ab1076 for cynomolgus monkey BMP9 in the pSMAD2 assay ranged from 27 pM-42 pM and from 465 pM-1 nM for rat BMP9. Representative images are presented in FIG. 6.

### Example 8: Anti-BMP9 Antibodies Ab732 and Ab1076 are Efficacious in the Rat Sugen-Hypoxia Model of Pulmonary Hypertension

An efficacy study to evaluate the effects of Ab732 and Ab1076 on pulmonary hypertension, relative to an isotype control and an activin ligand trap (ActRIIA-Fc), in the pre-clinical Sugen-Hypoxia (SuHx) model of pulmonary arterial hypertension (PAH) was performed. In this vasculoproliferative model, rodents develop progressive obliterative and complex lesions reminiscent of severe human disease (Abe, K., Toba, M., Alzoubi, A., Ito, M., Fagan, K. A., Cool, C. D., Voelkel, N. F., McMurtry, I. F., and Oka, M. (2010) Formation of plexiform lesions in experimental severe pulmonary arterial hypertension. Circulation 121, 2747-2754).

Male Sprague-Dawley rats (150-200 g) received a single subcutaneous injection of the vascular endothelial growth factor receptor 1/2 antagonist Sugen 5416 (20 mg/kg) and were placed in normobaric hypoxia (FiO₂=0.1) for moderate disease induction. An age-matched control untreated (normoxic) group was also included. Four weeks later, SuHx-treated rats were returned to normoxia and randomized to different treatment groups with equivalent body weights.

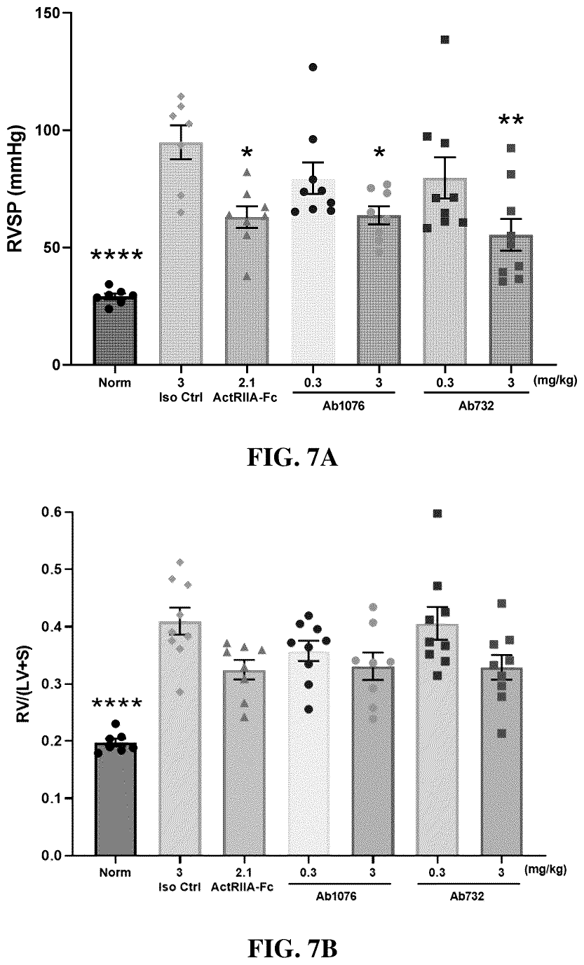

Treatments were administered intraperitoneally, twice per week, for a period of 3 weeks. Ab732 and Ab1076 were tested at 0.3 mg/kg and 3 mg/kg. Human ActRIIA-Fc protein was administered at 2.1 mg/kg, and an isotype control human IgG₁ at 3 mg/kg. At the end of the study, cardiovascular hemodynamic assessments were performed. Rats were anesthetized, and right ventricular systolic pressures (RVSP) were measured by catheterization. To assess the degree of right ventricular hypertrophy (RVH), the right ventricular free wall was dissected from the left ventricle plus septum (LV+S) in explanted hearts and weighed; the RVH was expressed as a ratio of weight of RV to weight of (LV+S). Results are shown in FIG. 7.

Mean RVSP and RVH in the untreated (normoxia) group were 29.2±1.25 mmHg and 0.197±0.00669, respectively. When administered after 4 weeks of SuHx, Ab1076 at 3 mg/kg significantly improved RVSP: 63.7±3.82 mmHg compared to 94.9±7.27 mmHg for the isotype control—a reduction of 31.2 mmHg or 47.4% of the [isotype control minus normoxia] difference (p<0.05). Ab1076 at 3 mg/kg also improved RVH (0.331±0.0240) relative to the isotype control (0.410±0.0236), representing a 37.1% decrease of the [isotype control minus normoxia] difference. Ab732 also induced significant improvements in the hemodynamic parameters: at 3 mg/kg, Ab732 significantly improved RVSP: 55.4±6.81 mmHg—a reduction of 39.5 mmHg or 60.0% of the [isotype control minus normoxia] difference (p<0.01). At 3 mg/kg, Ab732 improved RVH (0.329±0.0216) relative to the isotype control, a 37.9% decrease of the [isotype control minus normoxia] difference.

ActRIIA-Fc treatment also significantly improved RVSP (62.9±4.60 mmHg, a reduction of 31.9 mmHg vs isotype control; p<0.05) and improved RVH (0.325±0.0171) relative to isotype control.

In summary, administration of Ab732, Ab1076, and ActRIIA-Fc attenuated the magnitude of PAH developed in this study.

### Example 9: Evaluation of Total Serum BMP9 and BMP10 Concentrations in Sugen-Hypoxia (SuHx) Model

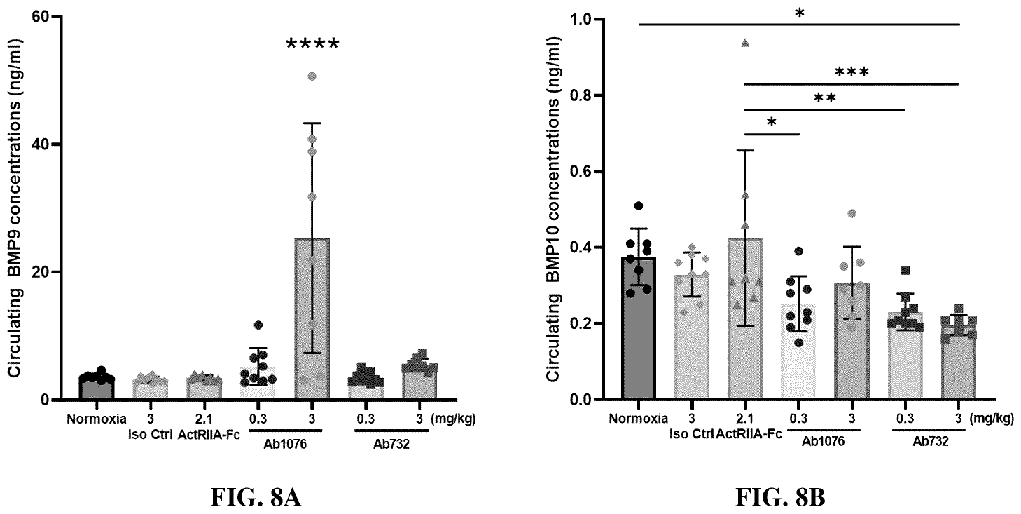

The effect of anti-BMP9 treatment on total soluble BMP9 and BMP10 concentrations in the rat serum samples collected at the end of the SuHx efficacy dose study of Example 8 was evaluated by immunoaffinity LCMS analysis. This functional readout was used for quantitative determination of target:antibody complex in serum, as a measure of target engagement. Results are shown in FIG. 8.

Similar and consistent circulating BMP9 and BMP10 concentrations were measured in serum from rats maintained in normoxic conditions and in SuHx rats treated with isotype control. No significant changes in BMP9 and BMP10 circulating concentrations were observed following treatment with ActRIIA-Fc.

A 1.7-fold increase in circulating BMP9 concentrations was observed upon treatment with 3 mg/kg of Ab732 relative to the normoxia and isotype control groups, and no changes were noted at 0.3 mg/kg. A dose-dependent accumulation of circulating BMP9 was observed with Ab1076 treatment: a 1.6-fold and a 7.9-fold increase was measured at 0.3 mg/kg and 3 mg/kg (p≤0.0001), respectively, relative to the normoxia and isotype control groups (FIG. 8A). This is consistent with a higher affinity of BMP9 to Ab1076 compared to Ab732. The magnitude of changes of circulating BMP10 are reduced relative to BMP9 (FIG. 8B).

### Example 10: Molecular Interactions of Ab93, Ab101 and Ab1076 with Human BMP9 Analyzed by X-Ray Crystallography

Crystallization and Structure Determination of Mature Human BMP9 in Complex with Ab1076 Fab

For crystallization trials, the complex between Ab1076 Fab and mature human BMP9 was formed at 1:1.5 molar ratio and was concentrated to 9.2 mg/ml in a protein solution containing TBS at pH 7.5.

The crystals were obtained by hanging-drop vapor-diffusion method from a condition containing 25% PEG 1500, 100 mM SPG buffer pH 5. The crystals had symmetry consistent with orthorhombic space group C222 with unit cell parameters a=141.98 Å; b=271.05 Å; c=62.59 Å and with two copies of BMP9+Ab1076 Fab complexes in the crystallographic asymmetric unit. The crystals were flash frozen in liquid nitrogen using 15% ethylene glycol (EG) as a cryoprotectant solution. A data set to a 2.55 Å resolution was collected from a single frozen crystal at IMCA beamline 17-ID at the Argonne National Laboratory. The data were processed and scaled using autoPROC, and the final data set was 62.7% complete.

The structure was solved by molecular replacement with PHASER. Several iterative rounds of manual adjustment and model rebuilding using COOT and crystallographic refinement using autoBUSTER yielded the final model of BMP9+Ab1076 Fab with a crystallographic R_{work} of 25.6% and R_{free} of 27.7%, where R_{work}=∥F_{obs}|−|F_{calc}∥/|F_{obs}| and R_{free} is equivalent to R_{work}, but calculated for a randomly chosen 5% of reflections omitted from the refinement process.

The BMP9 epitopes of antibodies Ab1076, Ab93, and Ab101 and of type II receptor ActRIIB are defined by structure-based descriptor method, outlined in detail below. The structure-based descriptor method utilizes the crystal structure complex and defines epitope residues as those residues that are: a) significantly buried by the complex formation (buried surface area of ≥20 Å²), b) residues participating in hydrogen bonds, c) residues participating in water-mediated hydrogen bonds, d) residues participating in salt bridges, and e) residues that are in close contact (<3.8 Å). This combination of interacting residues and close residues is then combined as the list of all potentially important epitope residues. This list is limited by the fact that sites that are not in close contact with the ligand can still be critical for binding and sites that are close may not be critical. The calculation used the H, L, and G chains from the Ab1076 structure, the H, L, and C chains from the Ab93 structure, the H, L, and I chains from the Ab101 structure, and chains A and E from Protein Data Bank entry 4fao to generate descriptors of interface residues.

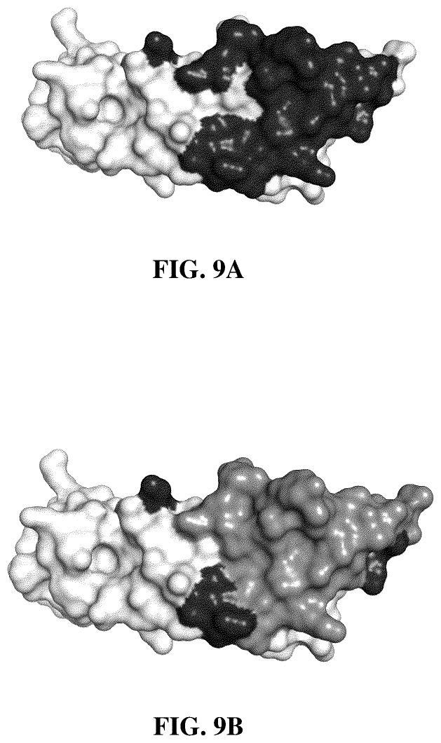

The BMP9 epitopes of antibodies Ab1076, Ab93, and Ab101, and of type II receptor ActRIIB, are summarized in Table 2. It is apparent from this analysis that there is a significant overlap in the epitopes for antibodies Ab1076, Ab93, and Ab101, and of type II receptor ActRIIB (FIG. 9), as well as other type II receptors such as BMPRII, ActRIIA, and endoglin (data not shown). The overlap of BMP9 epitope of antibody Ab1076 and ActRIIB is illustrated in FIG. 9.

TABLE 2
BMP9 epitopes of antibodies Ab1076, Ab93 and Ab101 and type II
receptor ActRIIB defined by structure-based descriptor method.

| Site | Amino acid | Ab1076 | Ab93 | Ab101 | ActRIIB |
|---|---|---|---|---|---|
| 14 | ARG | | | X | X |
| 27 | ILE | X | | X | X |
| 28 | ALA | X | X | X | X |
| 29 | PRO | X | X | X | X |
| 30 | LYS | X | | X | X |
| 31 | GLU | X | | X | X |
| 32 | TYR | X | | | X |
| 33 | GLU | | | | X |
| 78 | LYS | | | | X |
| 80 | SER | X | | | X |
| 81 | PRO | X | X | | X |
| 82 | ILE | X | | | |
| 83 | SER | X | X | X | X |
| 84 | VAL | X | | | X |
| 85 | LEU | X | X | X | X |
| 86 | TYR | X | X | | |
| 87 | LYS | | | | X |
| 88 | ASP | X | X | | |
| 89 | ASP | X | X | | |
| 90 | MET | X | X | X | |
| 91 | GLY | | | | X |
| 92 | VAL | X | X | X | X |
| 93 | PRO | X | X | X | X |
| 94 | THR | X | X | | X |
| 95 | LEU | X | X | X | X |
| 96 | LYS | X | X | | |
| 97 | TYR | X | X | X | X |
| 98 | HIS | X | X | | X |
| 99 | TYR | X | X | | |
| 100 | GLU | X | X | | |

### Structure-Based Epitope Residue Descriptors

Here, the H, L, and G chains from the Ab1076:BMP9 complex structure, the H, L, and C chains from the Ab93:BMP9 complex structure, and the H, L, and I chains from the Ab101:BMP9 complex structure were used to generate descriptors of interface residues. The residues of the antigen and antibody are said to be hydrogen bonded if they include a hydrogen bond donor atom (bound to an electropositive hydrogen) in one molecule located within 3.2 Å of a hydrogen bond acceptor atom having a lone pair of electrons in the other molecule. Additionally, a given residue was considered to be part of a water mediated hydrogen bond if it participates in a hydrogen bond with a water molecule that is also hydrogen bonded to the antibody. Residues of the antibody and antigen are said to form a salt bridge if they contain a positively charged atom in one molecule within 4 Å of a negatively charged atom in the other molecule. Residues are in close contact if one atom in the antigen residue is within 3.8 Å of one atom in the antibody.

The per-residue buried surface area was determined by calculating the solvent accessible surface area of each residue of the antibody and antigen in complex and subtracting this from the sum of the solvent accessible surface areas of the two components considered individually. The solvent accessible surface area was calculated according to the method of Strake and Rupley (J. Mol. Biol. 79 (2): 351-71, 1973). The pairwise buried surface area was used to estimate the individual contributions of pairs of residues from the antibody and antigen to the overall effect of buried surface area on binding energy. Since buried surface area is not pairwise decomposable, the buried surface area of each residue in the epitope was calculated in the presence of each individual antigen residue in the absence of the rest of the antigen. These individual contributions were then normalized so that the sum of all individual contributions of all antigen residues to the buried surface area of a given epitope residue would equal the total buried surface area of that epitope residue due to the binding of the entire antigen. This process was repeated in reverse for the individual contributions of epitope residues to the buried surface area of antibody residues. The antigen residue was considered to be part of the epitope if the buried surface area was ≥20 Å².

The same calculation was performed on the interaction of the binding epitope of BMP9 for ActRIIB using chains A and E from Protein Data Base structure 4fao.

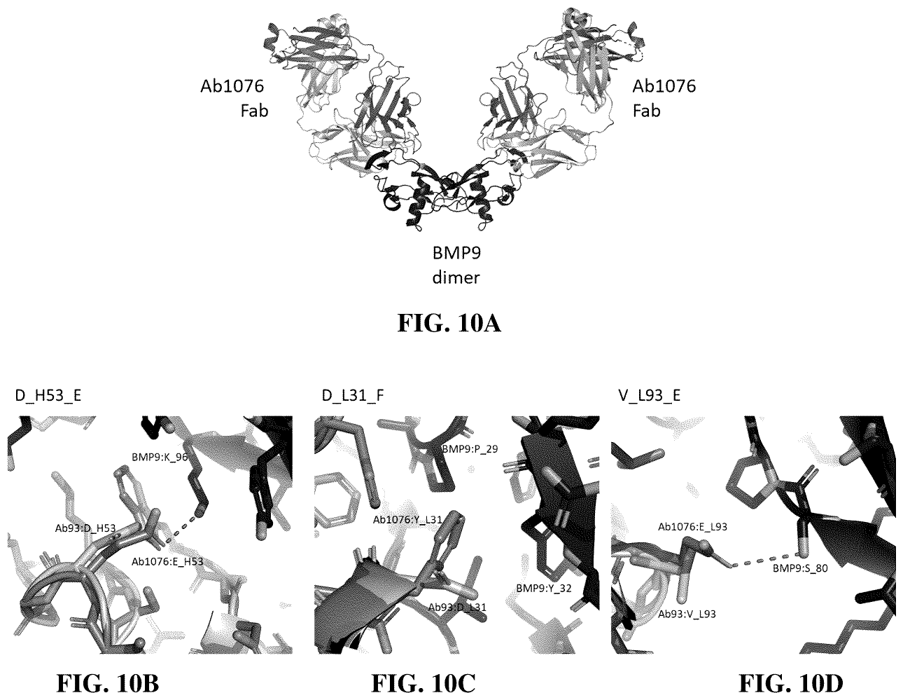

The binding interaction of Ab1076 Fab with human BMP9 in a crystal structure is illustrated in FIG. 10.

### Example 11: Anti-BMP9 Antibodies Inhibit Serum-Induced Phospho-SMAD1/5/9 and Phospho-SMAD2 Signaling in Human Endothelial Cells

Rat serum samples collected at the end of the SuHx efficacy dose study of Example 8 were used to evaluate the effect of anti-BMP9 and ActRIIA-Fc treatments on pSMAD1/5/9 and pSMAD2 nuclear translocation in immortalized human microvascular endothelial cells. Briefly, serum-starved TIME cells were treated with 10% rat serum for 20 minutes. The cells were fixed, blocked and permeabilized, and stained overnight with an antibody against pSMAD1/5/9 or pSMAD2. The next day, the cells were washed and stained with a secondary antibody labeled with ALEXA FLUOR® 488 to detect pSMAD1/5/9 or pSMAD2, Hoechst 33342 to detect nuclei, and a cell mask deep red stain to delineate whole cells. After a final wash, the cells were imaged on a high content imager. To analyze pSMAD1/5/9 or pSMAD2 nuclear translocation, the average nuclear fluorescence in the ALEXA FLUOR® 488 channel was calculated for each well of cells. IC₅₀ values were calculated using GraphPad Prism Version 9.0.0.

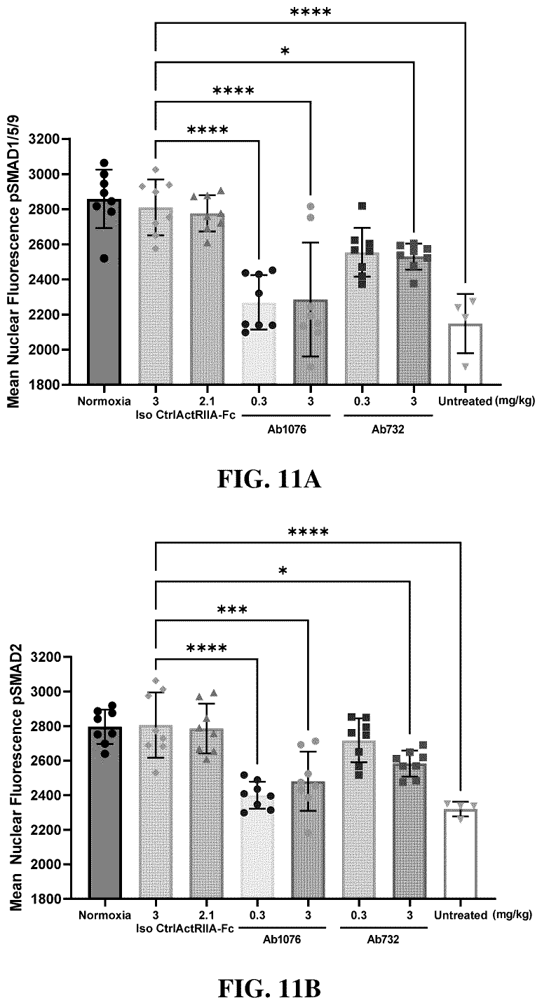

Serum from rats treated with 0.3 mg/kg of Ab1076 induced significantly less pSMAD1/5/9 mean nuclear fluorescence intensity: 2270±155 compared to 2810±160 for the Iso Ctrl, a reduction of 19.3% (p≤0.0001). A reduction of 18.7% in pSMAD1/5/9 nuclear intensity was observed upon treatment with 3 mg/kg of Ab1076 (2290±325) relative to the Iso Ctrl (p≤0.0001). Treatment with serum from Ab732-treated rats at 0.3 mg/kg (2550±140) and 3 mg/kg (2530±74.6; p≤0.05) inhibited pSMAD1/5/9 mean nuclear fluorescence relative to serum from rats treated with Iso Ctrl, a reduction of 9.2% and 10% respectively (FIG. 11A).

The average mean pSMAD2 nuclear intensity was reduced by 14.5% upon treatment with serum from the 0.3 mg/kg Ab1076 group (2400±79.1; p≤0.0001) and by 11.6% (2481±171; p=0.0002) upon treatment with serum from the 3 mg/kg Ab1076 group compared to the Iso Ctrl (2810±189). Treatment with serum from Ab732-treated rats at 0.3 mg/kg (2720±127) and 3 mg/kg (2580±75.7; p≤0.05) inhibited pSMAD2 mean nuclear fluorescence relative to serum from rats treated with Iso Ctrl (FIG. 11B).

Similar levels of pSMAD1/5/9 (2150±169) and pSMAD2 (2320±42.4) nuclear translocation were observed in untreated TIME endothelial cells and in cells treated with serum from the 0.3 mg/kg or 3 mg/kg Ab1076 groups.

No significant changes in pSMAD1/5/9 (2780±104) and pSMAD2 (2790±144) nuclear translocation were observed upon treatment with ActRIIA-Fc relative to the Iso Ctrl.

### Example 12: Evaluation of Off-Target Effects of Anti-BMP9 Antibodies on TGFβ-Family Members GDF8- and GDF11-Induced Signaling

The ability of Ab1076 and Ab732 to inhibit the activity of BMP9 family members GDF8 and GDF11 was assessed using C2C12 mouse myoblasts stably expressing the CAGA promoter inserted into a firefly luciferase reporter construct. Briefly, C2C12-CagaLuc cells were treated with 3 nM GDF8 or GDF11 in the presence of a range of doses of Ab1076, Ab732, or an isotype control antibody for 24 hours. Luciferase activity was assessed by lysing the cells and adding a firefly-specific substrate D-luciferein, then measuring luminescence on a plate reader.

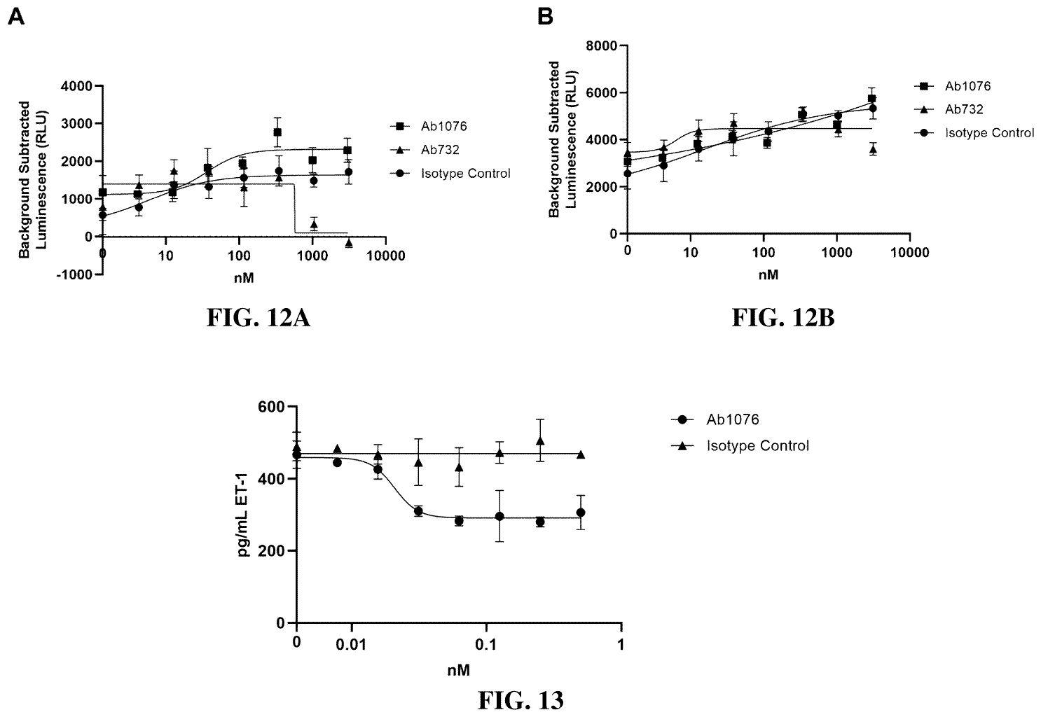

Ab1076 had no effect on either GDF8- (FIG. 12A) or GDF11- (FIG. 12B) induced luciferase activity relative to a non-binding isotype control antibody. Ab732 inhibited GDF8-induced luciferase activity (FIG. 12A), but not GDF11-induced luciferase activity (FIG. 12B). ActRIIA-Fc was used as a positive control and inhibited GDF8 and GDF11 with IC₅₀ values in the 0.2 nM-1.5 nM range (data not shown).

### Example 13: Evaluation of Effects of Anti-BMP9 Antibody Ab1076 on Inhibition of Endothelial Secretion of Endothelin-1

In this example, the ability of anti-BMP9 antibody Ab1076 to inhibit endothelial cell secretion of endothelin-1 (ET-1) was tested. Briefly, human pulmonary artery endothelial cells (HPAEC) were treated with 100 pM BMP9 in the presence of a range of doses of Ab1076 or isotype control for 24 hours. At the end of the treatment period, media was collected from each well of cells and subjected to ET-1 ELISA. A standard curve was generated using four parameter logistic curve fit, and this was used to determine the amount of ET-1 in each of the sample wells. IC₅₀ values were calculated using GraphPad Prism Version 9.0.0.

BMP9 stimulated an increase in ET-1 secretion (data not shown), and Ab1076 reduced BMP9-induced increases in ET-1 secretion with IC₅₀ values ranging from 0.021 nM-0.065 nM in several experiments performed (FIG. 13).

### Example 14: Identification of Gene and Protein Targets for Treatment of Pulmonary Hypertension with Anti-BMP9 Antibodies

In this example, potential target genes of BMP9 in pulmonary hypertension were identified.

Bulk RNAseq data from rat lungs treated with normoxia (Nx), SU5416+Hypoxia+Isotype control (SuHx_Iso), and SU5416+Hypoxia+Ab93 (SuHx_Ab93) was analyzed. The induction of gene expression and secretion of gene products correlated with pulmonary hypertension in response to BMP9 expression was also tested in HPMVEC.

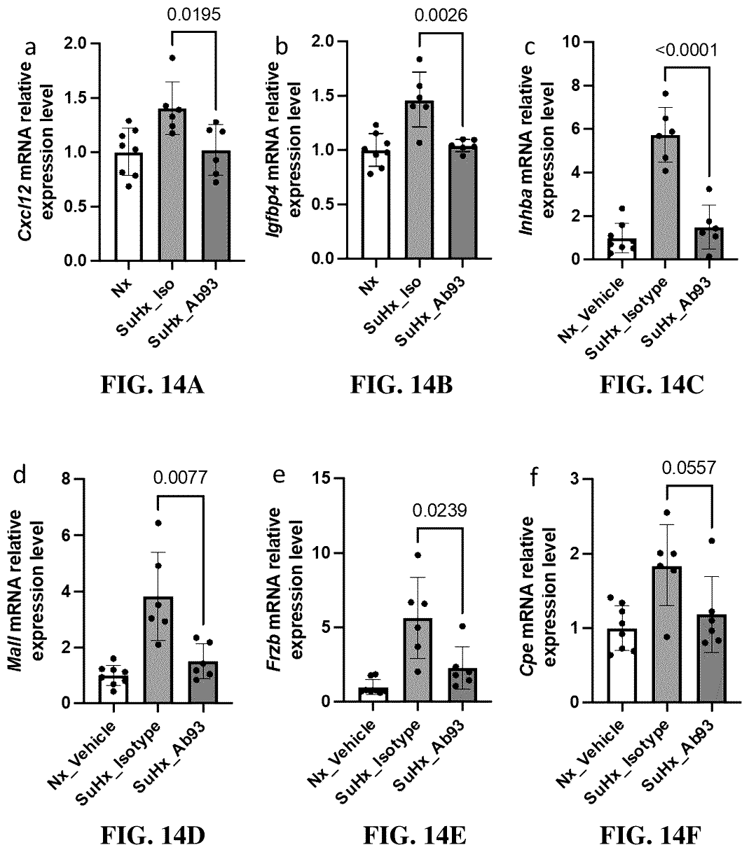

Paired-end raw reads in FASTQ format were aligned to the Rattus norvegicus rn7 reference genome using the Rsubread R package (2.8.2). FeatureCounts was used to count numbers of reads mapped to each gene. Differential expression analysis was performed using the DESeq2 R package (1.34.0). The relative mRNA expression level (fold change) for the following genes of interest was increased in SU5416+Hypoxia-treated (SuHx) rat lungs compared to normoxic (Nx) rat lungs and decreased in SuHx rat lungs treated with Ab93 and mapped to certain cell types as indicated below in human lung scRNAseq data (Saygin et al., Pulm Circ, 2020) (FIG. 14).
a. Cxcl12, C—X—C motif chemokine ligand 12, also named Stromal cell-derived factor 1 (SDF1) (FIG. 14A). Cxcl12 expression was increased in human IPAH lung endothelial cells and fibroblasts. The protein encoded by this gene is a secreted protein. In some aspects, neutralization of CXCL12 can attenuate established pulmonary hypertension in rats.
b. Igfbp4, Insulin-like growth factor binding protein 4 (FIG. 14B). Igfbp4 expression was increased in human IPAH lung endothelial cells and smooth muscle cells/pericytes. The protein encoded by this gene is a secreted protein. In some aspects, IGFBP4 inhibits angiogenesis.
c. Inhba, Inhibin, Beta A, also known as Activin A, Activin AB Alpha polypeptide, or Erythroid Differentiation protein (FIG. 14C). The protein encoded by this gene is a member of the TGF-beta superfamily of proteins. Increased expression of Activin A was mapped to SPP1 macrophages in human IPAH lungs, but it was decreased in FABP4 macrophages and proliferating cells (macrophages and ECs).
d. Mall, or Mal, T cell differentiation protein like (FIG. 14D). Increased expression of MALL was mapped to epithelial cells in human IPAH lungs.
e. Frzb, secreted Frizzled-related protein 3 (FIG. 14E). The protein encoded by this gene is a secreted protein, which is involved in bone development regulation. Increased expression of FRZB was mapped to SMC/pericytes in human IPAH lungs.
f. Cpe, Carboxypeptidase E (FIG. 14F). Cpe encodes a member of the M14 family of metallocarboxypeptidases. The encoded protein is a secreted protein. In some aspects, CPE upregulates the expression of anti-apoptotic protein Bc1-2 and inhibits caspase-3 activation. Increased expression of CPE was mapped to SMC/pericytes in human IPAH lungs.

Accordingly, among the genes that were upregulated in SuHx_Iso rat lungs compared to Nx rat lungs but downregulated in SuHx-Ab93 rat lungs compared to SuHx_Iso rat lungs, Cxcl12, Igfbp4, Inhba, Mall, Frzb, and Cpe were selected as potential target genes of BMP9. The expression of these genes has also been shown to be upregulated in certain cell types of IPAH patient lungs, as tested by scRNAseq (Saygin et al., Pulm. Circ., 2020). Among the six genes, expression of Cxcl12, Inhba, Mall, Frzb, and Cpe was also shown to be upregulated in PAH patient lungs, as tested by microarray assay (Stearman et al., Am J Respir Cell Mol Biol, 2019).

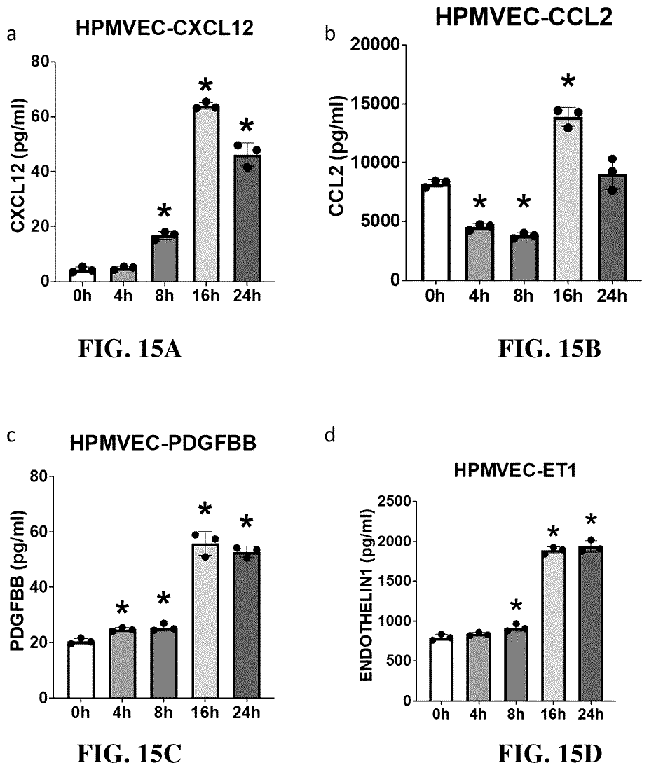

Low passage (<3) cultured human pulmonary microvascular endothelial cells (HPMVEC, Lonza) were treated with BMP9 (40 pM) for 0, 4, 8, 16, or 24 hours, and culture supernatants were analyzed using the Ella Automated Immunoassay System for human CXCL12 (protein simple: SPCKB-PS-000299) (FIG. 15A); Human CCL2 (FIG. 15B); PDGF-BB (protein simple: SPCKC-PS-006510) (FIG. 15C); and Human Endothelin-1 (also known as ET-1) (protein simple: SPCKC-PS-000265) (FIG. 15D). The concentration of CXCL12, CCL2, PDGFBB, and Endothelin) proteins in HPMVEC culture medium was increased by BMP9. CXCL12 was identified by RNAseq analysis. CCL2, C—C chemokine ligand type 2, was reported to be overexpressed in PAH, and inhibition of CCL2 decreases PAH in animals (Yu et al., Am J Respir Cell Mol Biol, 2013). PDGFBB and Endothelin-1 (encoded by Edn1) have been reported to be involved in PAH.

Multiple target genes of BMP9 were identified using in vivo RNAseq data analysis and in vitro cell culture media analysis. Some of the genes were shown to be strongly correlated with pulmonary hypertension, including Cxcl12, Ccl2, Pdgfb, and Edn1. These genes are potential biomarkers of pulmonary hypertension. In some aspects, the gene products may be targeted by one or more of the anti-BMP9 antibodies, or antigen-binding fragments, or pharmaceutical compositions thereof, disclosed herein for treatment of pulmonary hypertension.

TABLE 3
Summary of antibody SEQ ID Nos.

| | Heavy Chain (HC) | | | | | | | | | | Light Chain (LC) | | | | | | |
|---|---|---|---|---|---|---|---|---|---|---|---|---|---|---|---|---|---|
| | HC | HC | HC | | | | | | | | LC | LC | LC | | | | |
| Antibody | CDR1 | CDR2 | CDR3 | JH | V_{H} | CH1 | HINGE | CH2 | CH3 | HC | CDR1 | CDR2 | CDR3 | JL | V_{L} | CL | LC |
| Ab89 | 9 | 10 | 11 | 17 | 16 | 12 | 13 | 14 | 15 | 8 | 2 | 3 | 4 | 7 | 6 | 5 | 1 |
| Ab93 | 24 | 25 | 26 | 28 | 27 | 12 | 13 | 14 | 15 | 23 | 19 | 20 | 21 | 7 | 22 | 5 | 18 |
| Ab100 | 36 | 37 | 38 | 17 | 39 | 12 | 13 | 14 | 15 | 35 | 30 | 31 | 32 | 34 | 33 | 5 | 29 |
| Ab101 | 45 | 46 | 47 | 17 | 48 | 12 | 13 | 14 | 15 | 44 | 2 | 41 | 42 | 7 | 43 | 5 | 40 |
| Ab352 | 53 | 54 | 26 | 28 | 55 | 12 | 13 | 14 | 15 | 52 | 50 | 20 | 21 | 7 | 51 | 5 | 49 |
| Ab732 | 45 | 61 | 62 | 17 | 63 | 12 | 13 | 14 | 15 | 60 | 57 | 41 | 58 | 7 | 59 | 5 | 56 |
| Ab804 | 53 | 70 | 26 | 28 | 71 | 12 | 13 | 14 | 15 | 69 | 65 | 66 | 67 | 7 | 68 | 5 | 64 |
| Ab1076 | 53 | 76 | 26 | 28 | 77 | 12 | 13 | 14 | 15 | 75 | 73 | 20 | 67 | 7 | 74 | 5 | 72 |

TABLE 4
Sequences. In the full HC sequences of all Ab clones, VH CDRs were marked to
show Kabat and Chotia designations: Kabat underlined, Chotia italicized. Only CDR-H1 and
CDR-H2 designations are different between Kabat and Chotia, but CDR-H3 is marked as well.
There is no difference between Kabat and Chotia in VL CDRs (not marked).

| SEQ | | |
|---|---|---|
| ID | Description | Sequence |
| 1 | Ab89_LC | NFMLTQPHSV SESPGKTVTI SCTGSSGSIA SNYVQWYQQR |
| | | PGSAPTTVIY EDNQRPSGVP DRESGSIDSS SNSASLTISG |
| | | LKTEDEADYY CQSYDSSIHV VFGGGTKLTV LGQPKAAPSV |
| | | TLFPPSSEEL QANKATLVCL ISDFYPGAVT VAWKADSSPV |
| | | KAGVETTTPS KQSNNKYAAS SYLSLTPEQW KSHRSYSCQV |
| | | THEGSTVEKT VAPTECS |
| 2 | Ab89_LC_CDR1 | TGSSGSIASN YVQ |
| 3 | Ab89_LC_CDR2 | EDNQRPS |
| 4 | Ab89_LC_CDR3 | QSYDSSIHVV |
| 5 | Ab89_CL | GQPKAAPSVT LFPPSSEELQ ANKATLVCLI SDFYPGAVTV |
| | | AWKADSSPVK AGVETTTPSK QSNNKYAASS YLSLTPEQWK |
| | | SHRSYSCQVT HEGSTVEKTV APTECS |
| 6 | Ab89_VL | NFMLTQPHSV SESPGKTVTI SCTGSSGSIA SNYVQWYQQR |
| | | PGSAPTTVIY EDNQRPSGVP DRESGSIDSS SNSASLTISG |
| | | LKTEDEADYY CQSYDSSIHV VFGGGTKLTV L |
| 7 | Ab89_JL | FGGGTKLTVL |
| 8 | Ab89_HC; can be | QVQLVQSGAE VKKPGASVKV SCKASGYTFT SYYMHWVRQA |
| | prepared with or | PGQGLEWMGI INPSGGSTSY AQKFQGRVTM TRDTSTSTVY |
| | without C-terminal | MELSSLRSED TAVYYCASRD YYGSGRVYYY |
| | lysine | YGMDVWGQGT TVTVSSASTK GPSVFPLAPS SKSTSGGTAA |
| | | LGCLVKDYFP EPVTVSWNSG ALTSGVHTFP AVLQSSGLYS |
| | | LSSVVTVPSS SLGTQTYICN VNHKPSNTKV DKKVEPKSCD |
| | | KTHTCPPCPA PEAAGAPSVF LFPPKPKDTL MISRTPEVTC |
| | | VVVDVSHEDP EVKFNWYVDG VEVHNAKTKP |
| | | REEQYNSTYR VVSVLTVLHQ DWLNGKEYKC KVSNKALPAP |
| | | IEKTISKAKG QPREPQVYTL PPSREEMTKN QVSLTCLVKG |
| | | FYPSDIAVEW ESNGQPENNY KTTPPVLDSD GSFFLYSKLT |
| | | VDKSRWQQGN VFSCSVMHEA LHNHYTQKSL SLSPG(K) |
| 9 | Ab89_HC_CDR1 | GYTFTSYYMH |
| 10 | Ab89_HC_CDR2 | IINPSGGSTS YAQKFQG |
| 11 | Ab89_HC_CDR3 | RDYYGSGRVY YYYGMDV |
| 12 | HC_CH1 | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSC |
| 13 | HC_HINGE | DKTHTCPPCP |
| 14 | HC_CH2 | APEAAGAPSV FLFPPKPKDT LMISRTPEVT CVVVDVSHED |
| | | PEVKFNWYVD GVEVHNAKTK PREEQYNSTY RVVSVLTVLH |
| | | QDWLNGKEYK CKVSNKALPA PIEKTISKAK |
| 15 | Ab89_HC_CH3 | GQPREPQVYT LPPSREEMTK NQVSLTCLVK GFYPSDIAVE |
| | | WESNGQPENN YKTTPPVLDS DGSFFLYSKL TVDKSRWQQG |
| | | NVFSCSVMHE ALHNHYTQKS LSLSPGK |
| 16 | Ab89_VH | QVQLVQSGAE VKKPGASVKV SCKASGYTFT |
| | | SYYMHWVRQA PGQGLEWMGI INPSGGSTSY |
| | | AQKFQGRVTM TRDTSTSTVY MELSSLRSED TAVYYCASRD |
| | | YYGSGRVYYY YGMDVWGQGT TVTVSS |
| 17 | Ab89_JH | WGQGTTVTVS S |
| 18 | Ab93_LC | QSVLTQPPSA SGTPGQRVTI SCSVTNSDID IDNVHWYQQL |
| | | PGMAPKLLFF HDYRPSGVSD RFSGSKSGTS ASLAIVGLQS |
| | | EDEGYYYCAG LDVAMRGPVF GGGTKLTVLG QPKAAPSVTL |
| | | FPPSSEELQA NKATLVCLIS DFYPGAVTVA WKADSSPVKA |
| | | GVETTTPSKQ SNNKYAASSY LSLTPEQWKS HRSYSCQVTH |
| | | EGSTVEKTVA PTECS |
| 19 | Ab93_LC_CDR1 | SVTNSDIDID NVH |
| 20 | Ab93_LC_CDR2 | HDYRPS |
| 21 | Ab93_LC_CDR3 | AGLDVAMRGP V |
| 22 | Ab93_VL | QSVLTQPPSA SGTPGQRVTI SCSVTNSDID IDNVHWYQQL |
| | | PGMAPKLLFF HDYRPSGVSD RFSGSKSGTS ASLAIVGLQS |
| | | EDEGYYYCAG LDVAMRGPVF GGGTKLTVL |
| 23 | Ab93_HC; can be | EVQLVESGGG LVKPGGSLRL SCAASGFTFS NAWLSWVRQA |
| | prepared with or | PGKGLEWVGR IKSKTDGGTT DYAAPVKGRF TISRDDSKNT |
| | without C-terminal | LYLQMNSLKT EDTAVYYCTT GTYWGQGTLV TVSSASTKGP |
| | lysine | SVFPLAPSSK STSGGTAALG CLVKDYFPEP VTVSWNSGAL |
| | | TSGVHTFPAV LQSSGLYSLS SVVTVPSSSL GTQTYICNVN |
| | | HKPSNTKVDK KVEPKSCDKT HTCPPCPAPE AAGAPSVFLF |
| | | PPKPKDTLMI SRTPEVTCVV VDVSHEDPEV KFNWYVDGVE |
| | | VHNAKTKPRE EQYNSTYRVV SVLTVLHQDW |
| | | LNGKEYKCKV SNKALPAPIE KTISKAKGQP REPQVYTLPP |
| | | SREEMTKNQV SLTCLVKGFY PSDIAVEWES NGQPENNYKT |
| | | TPPVLDSDGS FFLYSKLTVD KSRWQQGNVF SCSVMHEALH |
| | | NHYTQKSLSL SPG(K) |
| 24 | Ab93_HC_CDR1 | GFTFSNAWLS |
| 25 | Ab93_HC_CDR2 | RIKSKTDGGT TDYAAPVKG |
| 26 | Ab93_HC_CDR3 | GTY |
| 27 | Ab93_VH | EVQLVESGGG LVKPGGSLRL SCAASGFTFS NAWLSWVRQA |
| | | PGKGLEWVGR IKSKTDGGTT DYAAPVKGRF TISRDDSKNT |
| | | LYLQMNSLKT EDTAVYYCTT GTYWGQGTLV TVSS |
| 28 | Ab93_JH | WGQGTLVTVS S |
| 29 | Ab100_LC | QSALTQPASV SGSPGQSITI SCTGTSSDVG SYNLVSWYQQ |
| | | HPGKAPKLMI YEVSKRPSGV SDRFSGSKSA NTASLTISGL |
| | | QAEDEADYYC SSFSINGPSF VFGTGTKVTV LGQPKAAPSV |
| | | TLFPPSSEEL QANKATLVCL ISDFYPGAVT VAWKADSSPV |
| | | KAGVETTTPS KQSNNKYAAS SYLSLTPEQW KSHRSYSCQV |
| | | THEGSTVEKT VAPTECS |
| 30 | Ab100_LC_CDR1 | TGTSSDVGSY NLVS |
| 31 | Ab100_LC_CDR2 | EVSKRPS |
| 32 | Ab100_LC_CDR3 | SSFSINGPSF V |
| 33 | Ab100_VL | QSALTQPASV SGSPGQSITI SCTGTSSDVG SYNLVSWYQQ |
| | | HPGKAPKLMI YEVSKRPSGV SDRFSGSKSA NTASLTISGL |
| | | QAEDEADYYC SSFSINGPSF VFGTGTKVTV L |
| 34 | Ab100_JL | FGTGTKVTVL |
| 35 | Ab100_HC; can | QMQLVQSGAE VKKPGSSVKV SCKASGGTFS SYAISWVRQA |
| | be prepared with | PGQGLEWMGG IIPIFGTANY AQKFQGRVTI TADESTSTAY |
| | or without C- | MELSSLRSED TAVYYCARVQ LDLSYYYYYG MDVWGQGTTV |
| | terminal lysine | TVSSASTKGP SVFPLAPSSK STSGGTAALG CLVKDYFPEP |
| | | VTVSWNSGAL TSGVHTFPAV LQSSGLYSLS SVVTVPSSSL |
| | | GTQTYICNVN HKPSNTKVDK KVEPKSCDKT HTCPPCPAPE |
| | | AAGAPSVFLF PPKPKDTLMI SRTPEVTCVV VDVSHEDPEV |
| | | KFNWYVDGVE VHNAKTKPRE EQYNSTYRVV |
| | | SVLTVLHQDW LNGKEYKCKV SNKALPAPIE KTISKAKGQP |
| | | REPQVYTLPP SREEMTKNQV SLTCLVKGFY PSDIAVEWES |
| | | NGQPENNYKT TPPVLDSDGS FFLYSKLTVD KSRWQQGNVF |
| | | SCSVMHEALH NHYTQKSLSL SPG(K) |
| 36 | Ab100_HC_CDR1 | GGTFSSYAIS |
| 37 | Ab100_HC_CDR2 | GIIPIFGTAN YAQKFQG |
| 38 | Ab100_HC_CDR3 | VQLDLSYYYY YGMDV |
| 39 | Ab100_VH | QMQLVQSGAE VKKPGSSVKV SCKASGGTFS SYAISWVRQA |
| | | PGQGLEWMGG IIPIFGTANY AQKFQGRVTI TADESTSTAY |
| | | MELSSLRSED TAVYYCARVQ LDLSYYYYYG |
| | | MDVWGQGTTV TVSS |
| 40 | Ab101_LC | NFMLTQPHSV SESPGKTVTI SCTGSSGSIA SNYVQWYQQR |
| | | PGSAPTTVIY EDKQRPSGVP DRFSGSIDRS SNSASLTISG |
| | | LKTEDEADYY CQSYDNSLSV VFGGGTKLTV LGQPKAAPSV |
| | | TLFPPSSEEL QANKATLVCL ISDFYPGAVT VAWKADSSPV |
| | | KAGVETTTPS KQSNNKYAAS SYLSLTPEQW KSHRSYSCQV |
| | | THEGSTVEKT VAPTECS |
| 41 | Ab101_LC_CDR2 | EDKQRPS |
| 42 | Ab101_LC_CDR3 | QSYDNSLSVV |
| 43 | Ab101_VL | NFMLTQPHSV SESPGKTVTI SCTGSSGSIA SNYVQWYQQR |
| | | PGSAPTTVIY EDKQRPSGVP DRFSGSIDRS SNSASLTISG |
| | | LKTEDEADYY CQSYDNSLSV VFGGGTKLTV L |
| 44 | Ab101_HC; can | QVQLVQSGAE VKKPGASVKV SCKASGYTFT SYGISWVRQA |
| | be prepared with | PGQGLEWMGW ISAYNGNTNY AQKLQGRVTM TTDTSTSTAY |
| | or without C- | MELRSLRSDD TAVYYCARAY GMDVWGQGTT VTVSSASTKG |
| | terminal lysine | PSVFPLAPSS KSTSGGTAAL GCLVKDYFPE PVTVSWNSGA |
| | | LTSGVHTFPA VLQSSGLYSL SSVVTVPSSS LGTQTYICNV |
| | | NHKPSNTKVD KKVEPKSCDK THTCPPCPAP EAAGAPSVFL |
| | | FPPKPKDTLM ISRTPEVTCV VVDVSHEDPE VKFNWYVDGV |
| | | EVHNAKTKPR EEQYNSTYRV VSVLTVLHQD |
| | | WLNGKEYKCK VSNKALPAPI EKTISKAKGQ PREPQVYTLP |
| | | PSREEMTKNQ VSLTCLVKGF YPSDIAVEWE SNGQPENNYK |
| | | TTPPVLDSDG SFFLYSKLTV DKSRWQQGNV FSCSVMHEAL |
| | | HNHYTQKSLS LSPG(K) |
| 45 | Ab101_HC_CDR1 | GYTFTSYGIS |
| 46 | Ab101_HC_CDR2 | WISAYNGNTN YAQKLQG |
| 47 | Ab101_HC_CDR3 | AYGMDV |
| 48 | Ab101_VH | QVQLVQSGAE VKKPGASVKV SCKASGYTFT SYGISWVRQA |
| | | PGQGLEWMGW ISAYNGNTNY AQKLQGRVTM |
| | | TTDTSTSTAY MELRSLRSDD TAVYYCARAY |
| | | GMDVWGQGTT VTVSS |
| 49 | Ab352_LC | QSVLTQPPSA SGTPGQRVTI SCSVTNSDID IFNVHWYQQL |
| | | PGTAPKLLFF HDYRPSGVSD RFSGSKSGTS ASLAISGLRS |
| | | EDEADYYCAG LDVAMRGPVF GGGTKLTVLG QPKAAPSVTL |
| | | FPPSSEELQA NKATLVCLIS DFYPGAVTVA WKADSSPVKA |
| | | GVETTTPSKQ SNNKYAASSY LSLTPEQWKS HRSYSCQVTH |
| | | EGSTVEKTVA PTECS |
| 50 | Ab352_LC_CDR1 | SVTNSDIDIF NVH |
| 51 | Ab352_VL | QSVLTQPPSA SGTPGQRVTI SCSVTNSDID IFNVHWYQQL |
| | | PGTAPKLLFF HDYRPSGVSD RFSGSKSGTS ASLAISGLRS |
| | | EDEADYYCAG LDVAMRGPVF GGGTKLTVL |
| 52 | Ab352_HC; can | EVQLVESGGG LVQPGGSLRL SCAASGFTFS NAWMSWVRQA |
| | be prepared with | PGKGLEWVGR IKSKTDSGTT DYAAPVKGRF TISRDNAKNS |
| | or without C- | LYLQMNSLRA EDTAVYYCTT GTYWGQGTLV TVSSASTKGP |
| | terminal lysine | SVFPLAPSSK STSGGTAALG CLVKDYFPEP VTVSWNSGAL |
| | | TSGVHTFPAV LQSSGLYSLS SVVTVPSSSL GTQTYICNVN |
| | | HKPSNTKVDK KVEPKSCDKT HTCPPCPAPE AAGAPSVFLF |
| | | PPKPKDTLMI SRTPEVTCVV VDVSHEDPEV KFNWYVDGVE |
| | | VHNAKTKPRE EQYNSTYRVV SVLTVLHQDW |
| | | LNGKEYKCKV SNKALPAPIE KTISKAKGQP REPQVYTLPP |
| | | SREEMTKNQV SLTCLVKGFY PSDIAVEWES NGQPENNYKT |
| | | TPPVLDSDGS FFLYSKLTVD KSRWQQGNVF SCSVMHEALH |
| | | NHYTQKSLSL SPG(K) |
| 53 | Ab352_HC_CDR1 | GFTFSNAWMS |
| 54 | Ab352_HC_CDR2 | RIKSKTDSGT TDYAAPVKG |
| 55 | Ab352_VH | EVQLVESGGG LVQPGGSLRL SCAASGFTFS NAWMSWVRQA |
| | | PGKGLEWVGR IKSKTDSGTT DYAAPVKGRF TISRDNAKNS |
| | | LYLQMNSLRA EDTAVYYCTT GTYWGQGTLV TVSS |
| 56 | Ab732_LC | NFMLTQPHSV SESPGKTVTI SCTGSSGNIA SNYVQWYQQR |
| | | PGSAPTTVIY EDKQRPSGVP DRFSGSIDRS SNSASLTISG |
| | | LKTEDEADYY CQSYDNHLSV VFGGGTKLTV LGQPKAAPSV |
| | | TLFPPSSEEL QANKATLVCL ISDFYPGAVT VAWKADSSPV |
| | | KAGVETTTPS KQSNNKYAAS SYLSLTPEQW KSHRSYSCQV |
| | | THEGSTVEKT VAPTECS |
| 57 | Ab732_LC_CDR1 | TGSSGNIASN YVQ |
| 58 | Ab732_LC_CDR3 | QSYDNHLSVV |
| 59 | Ab732_VL | NFMLTQPHSV SESPGKTVTI SCTGSSGNIA SNYVQWYQQR |
| | | PGSAPTTVIY EDKQRPSGVP DRFSGSIDRS SNSASLTISG |
| | | LKTEDEADYY CQSYDNHLSV VFGGGTKLTV L |
| 60 | Ab732_HC; can | QVQLVQSGAE VKKPGASVKV SCKASGYTFT SYGISWVRQA |
| | be prepared with | PGQGLEWMGW ISAYIGNTNY AQKLQGRVTM TTDTSISTAY |
| | or without C- | MELSRLRSDD TAVYYCARAY YMDVWGQGTT VTVSSASTKG |
| | terminal lysine | PSVFPLAPSS KSTSGGTAAL GCLVKDYFPE PVTVSWNSGA |
| | | LTSGVHTFPA VLQSSGLYSL SSVVTVPSSS LGTQTYICNV |
| | | NHKPSNTKVD KKVEPKSCDK THTCPPCPAP EAAGAPSVFL |
| | | FPPKPKDTLM ISRTPEVTCV VVDVSHEDPE VKFNWYVDGV |
| | | EVHNAKTKPR EEQYNSTYRV VSVLTVLHQD |
| | | WLNGKEYKCK VSNKALPAPI EKTISKAKGQ PREPQVYTLP |
| | | PSREEMTKNQ VSLTCLVKGF YPSDIAVEWE SNGQPENNYK |
| | | TTPPVLDSDG SFFLYSKLTV DKSRWQQGNV FSCSVMHEAL |
| | | HNHYTQKSLS LSPG(K) |
| 61 | Ab732_HC_CDR2 | WISAYIGNTN YAQKLQG |
| 62 | Ab732_HC_CDR3 | AYYMDV |
| 63 | Ab732_VH | QVQLVQSGAE VKKPGASVKV SCKASGYTFT SYGISWVRQA |
| | | PGQGLEWMGW ISAYIGNTNY AQKLQGRVTM TTDTSISTAY |
| | | MELSRLRSDD TAVYYCARAY YMDVWGQGTT VTVSS |
| 64 | Ab804_LC | QSVLTQPPSA SGTPGQRVTI SCSVTNPDID IFNVHWYQQL |
| | | PGTAPKLLFF HEYRPSGVSD RFSGSKSGTS ASLAIVGLQS |
| | | EDEGYYYCAG LDEAMRGPVF GGGTKLTVLG QPKAAPSVTL |
| | | FPPSSEELQA NKATLVCLIS DFYPGAVTVA WKADSSPVKA |
| | | GVETTTPSKQ SNNKYAASSY LSLTPEQWKS HRSYSCQVTH |
| | | EGSTVEKTVA PTECS |
| 65 | Ab804_LC_CDR1 | SVTNPDIDIF NVH |
| 66 | Ab804_LC_CDR2 | HEYRPS |
| 67 | Ab804_LC_CDR3 | AGLDEAMRGP V |
| 68 | Ab804_VL | QSVLTQPPSA SGTPGQRVTI SCSVTNPDID IFNVHWYQQL |
| | | PGTAPKLLFF HEYRPSGVSD RFSGSKSGTS ASLAIVGLQS |
| | | EDEGYYYCAG LDEAMRGPVF GGGTKLTVL |
| 69 | Ab804_HC; can | EVQLVESGGG LVKPGGSLRL SCAASGFTFS NAWMSWVRQA |
| | be prepared with | PGKGLEWVGR IKSKTDSGTT DYAAPVEGRF TISRDDSKNT |
| | or without C- | LYLQMNSLKT EDTAVYYCTT GTYWGQGTLV TVSSASTKGP |
| | terminal lysine | SVFPLAPSSK STSGGTAALG CLVKDYFPEP VTVSWNSGAL |
| | | TSGVHTFPAV LQSSGLYSLS SVVTVPSSSL GTQTYICNVN |
| | | HKPSNTKVDK KVEPKSCDKT HTCPPCPAPE AAGAPSVFLF |
| | | PPKPKDTLMI SRTPEVTCVV VDVSHEDPEV KFNWYVDGVE |
| | | VHNAKTKPRE EQYNSTYRVV SVLTVLHQDW |
| | | LNGKEYKCKV SNKALPAPIE KTISKAKGQP REPQVYTLPP |
| | | SREEMTKNQV SLTCLVKGFY PSDIAVEWES NGQPENNYKT |
| | | TPPVLDSDGS FFLYSKLTVD KSRWQQGNVF SCSVMHEALH |
| | | NHYTQKSLSL SPG(K) |
| 70 | Ab804_HC_CDR2 | RIKSKTDSGT TDYAAPVEG |
| 71 | Ab804_VH | EVQLVESGGG LVKPGGSLRL SCAASGFTFS NAWMSWVRQA |
| | | PGKGLEWVGR IKSKTDSGTT DYAAPVEGRF TISRDDSKNT |
| | | LYLQMNSLKT EDTAVYYCTT GTYWGQGTLV TVSS |
| 72 | Ab1076_LC | QSVLTQPPSA SGTPGQRVTI SCSVTNTDID IFNVHWYQQL |
| | | PGTAPKLLFF HDYRPSGVSD RFSGSKSGTS ASLAIVGLQS |
| | | EDEGYYYCAG LDEAMRGPVF GGGTKLTVLG QPKAAPSVTL |
| | | FPPSSEELQA NKATLVCLIS DFYPGAVTVA WKADSSPVKA |
| | | GVETTTPSKQ SNNKYAASSY LSLTPEQWKS HRSYSCQVTH |
| | | EGSTVEKTVA PTECS |
| 73 | Ab1076_LC_CDR1 | SVTNTDIDIF NVH |
| 74 | Ab1076_VL | QSVLTQPPSA SGTPGQRVTI SCSVTNTDID IFNVHWYQQL |
| | | PGTAPKLLFF HDYRPSGVSD RFSGSKSGTS ASLAIVGLQS |
| | | EDEGYYYCAG LDEAMRGPVF GGGTKLTVL |
| 75 | Ab1076_HC; can | EVQLVESGGG LVKPGGSLRL SCAASGFTFS NAWMSWVRQA |
| | be prepared with | PGKGLEWVGR IKSKTEGGDT DYAAPVKGRF TISRDDSKNT |
| | or without C- | LYLQMNSLKT EDTAVYYCTT GTYWGQGTLV TVSSASTKGP |
| | terminal lysine | SVFPLAPSSK STSGGTAALG CLVKDYFPEP VTVSWNSGAL |
| | | TSGVHTFPAV LQSSGLYSLS SVVTVPSSSL GTQTYICNVN |
| | | HKPSNTKVDK KVEPKSCDKT HTCPPCPAPE AAGAPSVFLF |
| | | PPKPKDTLMI SRTPEVTCVV VDVSHEDPEV KFNWYVDGVE |
| | | VHNAKTKPRE EQYNSTYRVV SVLTVLHQDW |
| | | LNGKEYKCKV SNKALPAPIE KTISKAKGQP REPQVYTLPP |
| | | SREEMTKNQV SLTCLVKGFY PSDIAVEWES NGQPENNYKT |
| | | TPPVLDSDGS FFLYSKLTVD KSRWQQGNVF SCSVMHEALH |
| | | NHYTQKSLSL SPG(K) |
| 76 | Ab1076_HC_CDR2 | RIKSKTEGGD TDYAAPVKG |
| 77 | Ab1076_VH | EVQLVESGGG LVKPGGSLRL SCAASGFTFS NAWMSWVRQA |
| | | PGKGLEWVGR IKSKTEGGDT DYAAPVKGRF TISRDDSKNT |
| | | LYLQMNSLKT EDTAVYYCTT GTYWGQGTLV TVSS |
| 78 | Human BMP9 | MCPGALWVALPLLSLLAGSLQGKPLQSWGRGSAGGNAHSPLG |
| | full-length protein. | VPGGGLPEHTFNLKMFLENVKVDFLRSLNLSGVPSQDKTRVE |
| | Italics denote | PPQYMIDLYNRYTSDKSTTPASNIVRSFSMEDAISITATEDFPF |
| | signal peptide; | QKHILLFNISIPRHEQITRAELRLYVSCQNHVDPSHDLKGSVVI |
| | Underlined | YDVLDGTDAWDSATETKTFLVSQDIQDEGWETLEVSSAVKR |
| | sequence denotes | WVRSDSTKSKNKLEVTVESHRKGCDTLDISVPPGSRNLPFFVV |
| | the prodomain; | FSNDHSSGTKETRLELREMISHEQESVLKKLSKDGSTEAGESS |
| | Mature sequence | HEEDTDGHVAAGSTLARRKRSAGAGSHCQKTSLRVNFEDIG |
| | shown in bold. | WDSWIIAPKEYEAYECKGGCFFPLADDVTPTKHAIVQTLV |
| | | HLKFPTKVGKACCVPTKLSPISVLYKDDMGVPTLKYHYE |
| | | GMSVAECGCR |
| 79 | Human BMP9 | SAGAGSHCQKTSLRVNFEDIGWDSWIIAPKEYEAYECKGGCF |
| | mature protein | FPLADDVTPTKHAIVQTLVHLKFPTKVGKACCVPTKLSPISVL |
| | (monomer) | YKDDMGVPTLKYHYEGMSVAECGCR |
| 80 | Ab93, Ab352, | GFTFSNAW(L/M)S |
| | Ab804, Ab1076 | |
| | VH CDR1 | |
| | Consensus | |
| 81 | Ab93, Ab352, | RIKSKT(D/E)(G/S)G(T/D)TDYAAPV(K/E)G |
| | Ab804, Ab1076 | |
| | VH CDR2 | |
| | Consensus | |
| 82 | Ab93, Ab352, | SVTN(S/P/T)DIDI(D/F)NVH |
| | Ab804, Ab1076 | |
| | VL CDR1 | |
| | Consensus | |
| 83 | Ab93, Ab352, | H(D/E)YRPS |
| | Ab804, Ab1076 | |
| | VL CDR2 | |
| | Consensus | |
| 84 | Ab93, Ab352, | AGLD(V/E)AMRGPV |
| | Ab804, Ab1076 | |
| | VL CDR3 | |
| | Consensus | |
| 85 | Ab1076 VH DNA | GAGGTGCAGCTGGTGGAGTCTGGGGGAGGCTTGGTAAAGC |
| | | CTGGGGGGTCCCTTAGACTCTCCTGTGCAGCCTCTGGATTC |
| | | ACTTTTAGTAACGCCTGGATGAGCTGGGTCCGCCAGGCTCC |
| | | AGGGAAGGGGCTGGAGTGGGTTGGCCGTATTAAAAGCAAA |
| | | ACTGAGGGTGGGGATACAGACTACGCTGCACCCGTGAAAG |
| | | GCAGATTCACCATCTCAAGAGATGATTCAAAAAACACGCT |
| | | GTATCTGCAAATGAACAGCCTGAAAACCGAGGACACAGCC |
| | | GTGTATTACTGTACCACAGGTACTTACTGGGGCCAGGGAAC |
| | | CCTGGTCACCGTCTCGAGC |
| 86 | Ab1076 VL DNA | CAGTCTGTGCTGACTCAGCCACCCTCAGCGTCTGGGACCCC |
| | | CGGCCAGAGGGTCACTATTTCTTGCTCTGTCACCAACACCG |
| | | ACATCGACATTTTCAATGTTCACTGGTATCAGCAGCTCCCA |
| | | GGAACGGCCCCCAAACTCCTCTTCTTTCATGATTACCGGCC |
| | | CTCAGGGGTCTCTGACCGATTTTCCGGCTCCAAGTCTGGAA |
| | | CCTCAGCCTCCCTGGCCATCGTTGGCCTCCAGTCTGAGGAT |
| | | GAGGGTTATTATTACTGTGCAGGATTGGATGAGGCTATGAG |
| | | GGGTCCGGTTTTCGGCGGCGGGACCAAGCTGACCGTCCTA |
| 87 | Ab1076 HC DNA | GAGGTGCAGCTGGTGGAGTCTGGGGGAGGCTTGGTAAAGC |
| | | CTGGGGGGTCCCTTAGACTCTCCTGTGCAGCCTCTGGATTC |
| | | ACTTTTAGTAACGCCTGGATGAGCTGGGTCCGCCAGGCTCC |
| | | AGGGAAGGGGCTGGAGTGGGTTGGCCGTATTAAAAGCAAA |
| | | ACTGAGGGTGGGGATACAGACTACGCTGCACCCGTGAAAG |
| | | GCAGATTCACCATCTCAAGAGATGATTCAAAAAACACGCT |
| | | GTATCTGCAAATGAACAGCCTGAAAACCGAGGACACAGCC |
| | | GTGTATTACTGTACCACAGGTACTTACTGGGGCCAGGGAAC |
| | | CCTGGTCACCGTCTCGAGCGCGTCGACCAAGGGCCCATCG |
| | | GTCTTCCCCCTGGCACCCTCCTCCAAGAGCACCTCTGGGGG |
| | | CACAGCGGCCCTGGGCTGCCTGGTCAAGGACTACTTCCCCG |
| | | AACCGGTGACGGTGTCGTGGAACTCAGGCGCCCTGACCAG |
| | | CGGCGTGCACACCTTCCCGGCTGTCCTACAGTCCTCAGGAC |
| | | TCTACTCCCTCAGCAGCGTGGTGACCGTGCCCTCCAGCAGC |
| | | TTGGGCACCCAGACCTACATCTGCAACGTGAATCACAAGC |
| | | CCAGCAACACCAAGGTGGACAAGAAAGTTGAGCCCAAATC |
| | | TTGTGACAAAACTCACACATGCCCACCGTGCCCAGCACCTG |
| | | AAGCCGCTGGGGCACCGTCAGTCTTCCTCTTCCCCCCAAAA |
| | | CCCAAGGACACCCTCATGATCTCCCGGACCCCTGAGGTCAC |
| | | ATGCGTGGTGGTGGACGTGAGCCACGAAGACCCTGAGGTC |
| | | AAGTTCAACTGGTACGTGGACGGCGTGGAGGTGCATAATG |
| | | CCAAGACAAAGCCGCGGGAGGAGCAGTACAACAGCACGT |
| | | ACCGTGTGGTCAGCGTCCTCACCGTCCTGCACCAGGACTGG |
| | | CTGAATGGCAAGGAGTACAAGTGCAAGGTCTCCAACAAAG |
| | | CCCTCCCAGCCCCCATCGAGAAAACCATCTCCAAAGCCAA |
| | | AGGGCAGCCCCGAGAACCACAGGTGTACACCCTGCCCCCA |
| | | TCCCGGGAGGAGATGACCAAGAACCAGGTCAGCCTGACCT |
| | | GCCTGGTCAAAGGCTTCTATCCCAGCGACATCGCCGTGGAG |
| | | TGGGAGAGCAATGGGCAGCCGGAGAACAACTACAAGACC |
| | | ACGCCTCCCGTGCTGGACTCCGACGGCTCCTTCTTCCTCTAT |
| | | AGCAAGCTCACCGTGGACAAGAGCAGGTGGCAGCAGGGG |
| | | AACGTCTTCTCATGCTCCGTGATGCATGAGGCTCTGCACAA |
| | | CCACTACACGCAGAAGAGCCTCTCCCTGTCCCCGGGAAAA |
| 88 | Ab1076 LC DNA | CAGTCTGTGCTGACTCAGCCACCCTCAGCGTCTGGGACCCC |
| | | CGGCCAGAGGGTCACTATTTCTTGCTCTGTCACCAACACCG |
| | | ACATCGACATTTTCAATGTTCACTGGTATCAGCAGCTCCCA |
| | | GGAACGGCCCCCAAACTCCTCTTCTTTCATGATTACCGGCC |
| | | CTCAGGGGTCTCTGACCGATTTTCCGGCTCCAAGTCTGGAA |
| | | CCTCAGCCTCCCTGGCCATCGTTGGCCTCCAGTCTGAGGAT |
| | | GAGGGTTATTATTACTGTGCAGGATTGGATGAGGCTATGAG |
| | | GGGTCCGGTTTTCGGCGGCGGGACCAAGCTGACCGTCCTA |
| | | GGTCAGCCCAAGGCTGCCCCCTCGGTCACTCTGTTCCCGCC |
| | | CTCCTCTGAGGAGCTTCAAGCCAACAAGGCCACACTGGTGT |
| | | GTCTCATAAGTGACTTCTACCCGGGAGCCGTGACAGTGGCC |
| | | TGGAAGGCAGATAGCAGCCCCGTCAAGGCGGGAGTGGAGA |
| | | CCACCACACCCTCCAAACAAAGCAACAACAAGTACGCGGC |
| | | CAGCAGCTATCTGAGCCTGACGCCTGAGCAGTGGAAGTCC |
| | | CACAGAAGCTACAGCTGCCAGGTCACGCATGAAGGGAGCA |
| | | CCGTGGAGAAGACAGTGGCCCCTACAGAATGTTCA |
| 89 | Antibody Light | MGWSCIILFLVATATGVHS |
| | chain and Heavy | |
| | chain signal | |
| | peptide | |

The foregoing description and Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the without departing from the concept, spirit and scope of the teachings herein and the claims appended below. While the present teachings have been described in terms of \ exemplary embodiments, the skilled artisan will readily understand that numerous variations and modifications of these exemplary embodiments are possible without undue experimentation. All such variations and modifications are deemed to be within the spirit, scope and concept of the disclosure as defined by the current teachings.

1. An isolated antibody that binds to bone morphogenetic protein-9 (BMP9) and comprises a heavy chain variable region (VH) and a light chain variable region (VL), wherein:
the VH complementarity determining region one (CDR1) comprises an amino acid sequence selected from the group consisting of SEQ ID NO: 9, SEQ ID NO: 24, SEQ ID NO: 36, SEQ ID NO: 45, and SEQ ID NO: 53,
the VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76,
the VH CDR3 comprises an amino acid sequence selected from the group consisting of SEQ ID NO: 11, SEQ ID NO: 26, SEQ ID NO: 38, SEQ ID NO: 47, and SEQ ID NO: 62, the VL CDR1 comprises an amino acid sequence selected from the group consisting of SEQ ID NO: 2, SEQ ID NO: 19, SEQ ID NO: 30, SEQ ID NO: 50, SEQ ID NO: 57, SEQ ID NO: 65, and SEQ ID NO: 73,
the VL CDR2 comprises an amino acid sequence selected from the group consisting of SEQ ID NO: 3, SEQ ID NO: 20, SEQ ID NO: 31, SEQ ID NO: 41, and SEQ ID NO: 66, and
the VL CDR3 comprises an amino acid sequence selected from the group consisting of SEQ ID NO: 4, SEQ ID NO: 21, SEQ ID NO: 32, SEQ ID NO: 42, SEQ ID NO: 58, and SEQ ID NO: 67.

2. An isolated antibody that binds to bone morphogenetic protein-9 (BMP9) and comprises a heavy chain variable region (VH) and a light chain variable region (VL), wherein:
(i) the VH CDR1 comprises the amino acid sequence of SEQ ID NO: 80, the VH CDR2 comprises the amino acid sequence of SEQ ID NO: 81, and the VH CDR3 comprises the amino acid sequence of SEQ ID NO: 26, and the VL CDR1 comprises the amino acid sequence of SEQ ID NO: 82, the VL CDR2 comprises the amino acid sequence of SEQ ID NO: 83, and the VL CDR3 comprises the amino acid sequence of SEQ ID NO: 84;
(ii) the VH CDR1 comprises the amino acid sequence of SEQ ID NO: 9, the VH CDR2 comprises the amino acid sequence of SEQ ID NO: 10, and the VH CDR3 comprises the amino acid sequence of SEQ ID NO: 11, and the VL CDR1 comprises the amino acid sequence of SEQ ID NO: 2, the VL CDR2 comprises the amino acid sequence of SEQ ID NO: 3, and the VL CDR3 comprises the amino acid sequence of SEQ ID NO: 4;
(iii) the VH CDR1 comprises the amino acid sequence of SEQ ID NO: 24, the VH CDR2 comprises the amino acid sequence of SEQ ID NO: 25, and the VH CDR3 comprises the amino acid sequence of SEQ ID NO: 26, and the VL CDR1 comprises the amino acid sequence of SEQ ID NO: 19, the VL CDR2 comprises the amino acid sequence of SEQ ID NO: 20, and the VL CDR3 comprises the amino acid sequence of SEQ ID NO: 21;
(iv) the VH CDR1 comprises the amino acid sequence of SEQ ID NO: 36, the VH CDR2 comprises the amino acid sequence of SEQ ID NO: 37, and the VH CDR3 comprises the amino acid sequence of SEQ ID NO: 38, and the VL CDR1 comprises the amino acid sequence of SEQ ID NO: 30, the VL CDR2 comprises the amino acid sequence of SEQ ID NO: 31, and the VL CDR3 comprises the amino acid sequence of SEQ ID NO: 32;
(v) the VH CDR1 comprises the amino acid sequence of SEQ ID NO: 45, the VH CDR2 comprises the amino acid sequence of SEQ ID NO: 46, and the VH CDR3 comprises the amino acid sequence of SEQ ID NO: 47, and the VL CDR1 comprises the amino acid sequence of SEQ ID NO: 2, the VL CDR2 comprises the amino acid sequence of SEQ ID NO: 41, and the VL CDR3 comprises the amino acid sequence of SEQ ID NO: 42;
(vi) the VH CDR1 comprises the amino acid sequence of SEQ ID NO: 53, the VH CDR2 comprises the amino acid sequence of SEQ ID NO: 54, and the VH CDR3 comprises the amino acid sequence of SEQ ID NO: 26, and the VL CDR1 comprises the amino acid sequence of SEQ ID NO: 50, the VL CDR2 comprises the amino acid sequence of SEQ ID NO: 20, and the VL CDR3 comprises the amino acid sequence of SEQ ID NO: 21;
(vii) the VH CDR1 comprises the amino acid sequence of SEQ ID NO: 45, the VH CDR2 comprises the amino acid sequence of SEQ ID NO: 61, and the VH CDR3 comprises the amino acid sequence of SEQ ID NO: 62, and the VL CDR1 comprises the amino acid sequence of SEQ ID NO: 57, the VL CDR2 comprises the amino acid sequence of SEQ ID NO: 41, and the VL CDR3 comprises the amino acid sequence of SEQ ID NO: 58;
(viii) the VH CDR1 comprises the amino acid sequence of SEQ ID NO: 53, the VH CDR2 comprises the amino acid sequence of SEQ ID NO: 70, and the VH CDR3 comprises the amino acid sequence of SEQ ID NO: 26, and the VL CDR1 comprises the amino acid sequence of SEQ ID NO: 65, the VL CDR2 comprises the amino acid sequence of SEQ ID NO: 66, and the VL CDR3 comprises the amino acid sequence of SEQ ID NO: 67; or
(ix) the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.

3. (canceled)

4. The isolated antibody of claim 2, wherein the VH CDR1 comprises the amino acid sequence of SEQ ID NO: 53, the VH CDR2 comprises the amino acid sequence of SEQ ID NO: 76, and the VH CDR3 comprises the amino acid sequence of SEQ ID NO: 26, and the VL CDR1 comprises the amino acid sequence of SEQ ID NO: 73, the VL CDR2 comprises the amino acid sequence of SEQ ID NO: 20, and the VL CDR3 comprises the amino acid sequence of SEQ ID NO: 67.

5. The isolated antibody of claim 2, wherein the VH comprises the amino acid sequence of SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, or a variant thereof comprising one to four amino acid substitutions at residues that are not within a CDR, and the VL comprises the amino acid sequence of SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74, or a variant thereof comprising one to four amino acid substitutions at residues that are not within a CDR.

6. The isolated antibody of claim 2, wherein the VH comprises an amino acid sequence at least 90%, at least 91%, at least 92%, at least 93%, at least 94%, at least 95%, at least 96%, at least 97%, at least 98%, at least 99%, or 100% identical to SEQ ID NO: 16, SEQ ID NO: 27, SEQ ID NO: 39, SEQ ID NO: 48, SEQ ID NO: 55, SEQ ID NO: 63, SEQ ID NO: 71, or SEQ ID NO: 77, and the VL comprises an amino acid sequence at least 90%, at least 91%, at least 92%, at least 93%, at least 94%, at least 95%, at least 96%, at least 97%, at least 98%, at least 99%, or 100% identical to SEQ ID NO: 6, SEQ ID NO: 22, SEQ ID NO: 33, SEQ ID NO: 44, SEQ ID NO: 51, SEQ ID NO: 59, SEQ ID NO: 68, or SEQ ID NO: 74.

7.-8. (canceled)

9. The isolated antibody of claim 2, wherein the antibody comprises a VH comprising the amino acid sequence of SEQ ID NO: 77 and a VL comprising the amino acid sequence of SEQ ID NO: 74.

10. A portion of an isolated antibody, wherein the isolated antibody binds to BMP9 and comprises a heavy chain variable region (VH) and a light chain variable region (VL), wherein:
the VH CDR1 comprises an amino acid sequence selected from the group consisting of SEQ ID NO: 9, SEQ ID NO: 24, SEQ ID NO: 36, SEQ ID NO: 45, and SEQ ID NO: 53;
the VH CDR2 comprises an amino acid sequence selected from the group consisting of SEQ ID NO: 10, SEQ ID NO: 25, SEQ ID NO: 37, SEQ ID NO: 46, SEQ ID NO: 54, SEQ ID NO: 61, SEQ ID NO: 70, and SEQ ID NO: 76;
the VH CDR3 comprises an amino acid sequence selected from the group consisting of SEQ ID NO: 11, SEQ ID NO: 26, SEQ ID NO: 38, SEQ ID NO: 47, and SEQ ID NO: 62;
the VL CDR1 comprises an amino acid sequence selected from the group consisting of SEQ ID NO: 2, SEQ ID NO: 19, SEQ ID NO: 30, SEQ ID NO: 50, SEQ ID NO: 57, SEQ ID NO: 65, and SEQ ID NO: 73;
the VL CDR2 comprises an amino acid sequence selected from the group consisting of SEQ ID NO: 3, SEQ ID NO: 20, SEQ ID NO: 31, SEQ ID NO: 41, and SEQ ID NO: 66; and
the VL CDR3 comprises an amino acid sequence selected from the group consisting of SEQ ID NO: 4, SEQ ID NO: 21, SEQ ID NO: 32, SEQ ID NO: 42, SEQ ID NO: 58, and SEQ ID NO: 67, and
wherein the portion binds to BMP9.

11. (canceled)

12. The isolated antibody of claim 2, wherein the isolated antibody comprises a VH encoded by the nucleic acid sequence of the insert of the plasmid deposited with the ATCC and having the Accession Number PTA-127292, a VL encoded by the nucleic acid sequence of the insert of the plasmid deposited with the ATCC and having the Accession Number PTA-127293, or both.

13. An isolated antibody comprising:
(i) the VH CDR1, VH CDR2, and VH CDR3 sequences of SEQ ID NO: 16 and the VL CDR1, VL CDR2, and VL CDR3 sequences of SEQ ID NO: 6;
(ii) the VH CDR1, VH CDR2, and VH CDR3 sequences of SEQ ID NO: 27 and the VL CDR1, VL CDR2, and VL CDR3 sequences of SEQ ID NO: 22;
(iii) the VH CDR1, VH CDR2, and VH CDR3 sequences of SEQ ID NO: 39 and the VL CDR1, VL CDR2, and VL CDR3 sequences of SEQ ID NO: 33;
(iv) the VH CDR1, VH CDR2, and VH CDR3 sequences of SEQ ID NO: 48 and the VL CDR1, VL CDR2, and VL CDR3 sequences of SEQ ID NO: 43;
(v) the VH CDR1, VH CDR2, and VH CDR3 sequences of SEQ ID NO: 55 and the VL CDR1, VL CDR2, and VL CDR3 sequences of SEQ ID NO: 51;
(vi) the VH CDR1, VH CDR2, and VH CDR3 sequences of SEQ ID NO: 63 and the VL CDR1, VL CDR2, and VL CDR3 sequences of SEQ ID NO: 59;
(vii) the VH CDR1, VH CDR2, and VH CDR3 sequences of SEQ ID NO: 71 and the VL CDR1, VL CDR2, and VL CDR3 sequences of SEQ ID NO: 68; or
(viii) the VH CDR1, VH CDR2, and VH CDR3 sequences of SEQ ID NO: 77 and the VL CDR1, VL CDR2, and VL CDR3 sequences of SEQ ID NO: 74.

14.-22. (canceled)

23. The isolated antibody of claim 2, wherein the antibody comprises:
(i) a heavy chain comprising an amino acid sequence selected from the group consisting of SEQ ID NO: 8, SEQ ID NO: 23, SEQ ID NO: 35, SEQ ID NO: 44, SEQ ID NO: 52, SEQ ID NO: 60, SEQ ID NO: 69, and SEQ ID NO: 75; and
(ii) a light chain comprising an amino acid sequence selected from the group consisting of SEQ ID NO: 1, SEQ ID NO: 18, SEQ ID No: 29, SEQ ID NO: 40, SEQ ID NO: 49, SEQ ID NO: 56, SEQ ID NO: 64, and SEQ ID NO: 72.

24. The isolated antibody of claim 2, wherein the antibody comprises:
(i) a heavy chain comprising an amino acid sequence at least 90%, at least 91%, at least 92%, at least 93%, at least 94%, at least 95%, at least 96%, at least 97%, at least 98%, at least 99%, or 100% identical to SEQ ID NO: 8, SEQ ID NO: 23, SEQ ID NO: 35, SEQ ID NO: 44, SEQ ID NO: 52, SEQ ID NO: 60, SEQ ID NO: 69, or SEQ ID NO: 75; and
(ii) a light chain comprising an amino acid sequence at least 90%, at least 91%, at least 92%, at least 93%, at least 94%, at least 95%, at least 96%, at least 97%, at least 98%, at least 99%, or 100% identical to SEQ ID NO: 1, SEQ ID NO: 18, SEQ ID No: 29, SEQ ID NO: 40, SEQ ID NO: 49, SEQ ID NO: 56, SEQ ID NO: 64, or SEQ ID NO: 72.

25.-26. (canceled)

27. The isolated antibody of claim 2, wherein the antibody comprises:
(i) a HC comprising, or consisting of, the amino acid sequence of SEQ ID NO: 75 and a LC comprising, or consisting of, the amino acid sequence of SEQ ID NO: 72 or
(ii) a HC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 75 and a LC comprising, or consisting of, an amino acid sequence at least 90%, at least 91%, at least 92%, at least 93%, at least 94%, at least 95%, at least 96%, at least 97%, at least 98%, at least 99%, or 100% identical to SEQ ID NO: 72.

28. An isolated antibody that binds to BMP9, wherein the antibody binds to an epitope on BMP9 comprising one or more amino acid residues selected from the group consisting of Ala28, Pro29, Ser83, Leu85, Met90, Val92, Pro93, Leu95, and Tyr97 of SEQ ID NO: 79.

29.-33. (canceled)

34. The isolated antibody of claim 28, wherein the antibody binds to an epitope on BMP9 comprising one or more amino acid residues selected from the group consisting of Ile27, Ala28, Pro29, Lys30, Glu31, Tyr32, Ser80, Pro81, Ile82, Ser83, Val84, Leu85, Tyr86, Asp88, Asp89, Met90, Val92, Pro93, Thr94, Leu95, Lys96, Tyr97, His98, Tyr99, and Glu100 of SEQ ID NO: 79.

35. (canceled)

36. The isolated antibody of claim 28, wherein the antibody binds to an epitope on BMP9 comprising one or more amino acid residues selected from the group consisting of Ala28, Pro29, Pro81, Ser83, Leu85, Tyr86, Asp88, Asp89, Met90, Val92, Pro93, Thr94, Leu95, Lys96, Tyr97, His98, Tyr99, and Glu100 of SEQ ID NO: 79.

37. (canceled)

38. An isolated antibody that binds to BMP9, wherein the antibody binds to an epitope on BMP9 comprising one or more amino acid residues selected from the group consisting of Arg14, Ile27, Ala28, Pro29, Lys30, Glu31, Ser83, Leu85, Met90, Val92, Pro93, Leu95, and Tyr97 of SEQ ID NO: 79.

39.-52. (canceled)

53. An isolated polynucleotide encoding the isolated antibody of claim 2.

54.-56. (canceled)

57. A vector comprising the polynucleotide of claim 53.

58. An isolated host cell comprising the polynucleotide of claim 53.

59.-60. (canceled)

61. A method of producing an antibody, comprising:
culturing the host cell of claim 58 under conditions that result in production of the antibody; and
recovering the antibody.

62. A pharmaceutical composition comprising a therapeutically effective amount of the antibody of claim 2 and a pharmaceutically acceptable carrier.

63.-66. (canceled)

67. A method for reducing the expression or activity of BMP9 in a subject, wherein the method comprises administering to a subject in need thereof a therapeutically effective amount of the isolated antibody of claim 2.

68. A method of treating hypertension in a subject, the method comprising administering to a subject in need thereof a therapeutically effective amount of the isolated antibody of claim 2.

69. (canceled)

70. A method of treating pulmonary arterial hypertension (PAH) in a subject, the method comprising administering to a subject in need thereof a therapeutically effective amount of the isolated antibody of claim 2.

71.-74. (canceled)

75. A method for monitoring the effectiveness of a pulmonary arterial hypertension (PAH) therapy in a subject in need thereof, the method comprising: (i) administering a PAH therapy to the subject; (ii) determining the level of one or more biomarkers in a biological sample obtained from the subject; and (iii) comparing the level of the one or more biomarkers from step (ii) to the level of the same one or more biomarkers before administration of the PAH therapy, wherein a change in the level of the one or more biomarkers after administration of the PAH therapy indicates that the therapy is effective, and wherein the PAH therapy comprises the isolated antibody of claim 2.

76. The method of claim 70, wherein the isolated antibody is administered at a dose of about 300 mg to about 400 mg.

77. The method of claim 70, wherein the isolated antibody is administered every 2 weeks, every 3 weeks, every 4 weeks, every 5 weeks, every 6 weeks, every 7 weeks, every 8 weeks, every 9 weeks, every 10 weeks, every 11 weeks, or every 12 weeks.


SEQUENCE LISTING (89 sequences)

SEQ ID NO: 1 - protein, 217 aa, synthetic construct
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSIHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 13 aa, synthetic construct
TGSSGSIASNYVQ

SEQ ID NO: 3 - protein, 7 aa, synthetic construct
EDNQRPS

SEQ ID NO: 4 - protein, 10 aa, synthetic construct
QSYDSSIHVV

SEQ ID NO: 5 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 111 aa, synthetic construct
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSIHVVFGGGTKLTVL

SEQ ID NO: 7 - protein, 10 aa, synthetic construct
FGGGTKLTVL

SEQ ID NO: 8 - protein, 456 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCASRDYYGSGRVYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 10 aa, synthetic construct
GYTFTSYYMH

SEQ ID NO: 10 - protein, 17 aa, synthetic construct
IINPSGGSTSYAQKFQG

SEQ ID NO: 11 - protein, 17 aa, synthetic construct
RDYYGSGRVYYYYGMDV

SEQ ID NO: 12 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 10 aa, synthetic construct
DKTHTCPPCP

SEQ ID NO: 14 - protein, 110 aa, synthetic construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCASRDYYGSGRVYYYYGMDVWGQGTTVTVSS

SEQ ID NO: 17 - protein, 11 aa, synthetic construct
WGQGTTVTVSS

SEQ ID NO: 18 - protein, 215 aa, synthetic construct
QSVLTQPPSASGTPGQRVTISCSVTNSDIDIDNVHWYQQLPGMAPKLLFFHDYRPSGVSDRFSGSKSGTSASLAIVGLQSEDEGYYYCAGLDVAMRGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 13 aa, synthetic construct
SVTNSDIDIDNVH

SEQ ID NO: 20 - protein, 6 aa, synthetic construct
HDYRPS

SEQ ID NO: 21 - protein, 11 aa, synthetic construct
AGLDVAMRGPV

SEQ ID NO: 22 - protein, 109 aa, synthetic construct
QSVLTQPPSASGTPGQRVTISCSVTNSDIDIDNVHWYQQLPGMAPKLLFFHDYRPSGVSDRFSGSKSGTSASLAIVGLQSEDEGYYYCAGLDVAMRGPVFGGGTKLTVL

SEQ ID NO: 23 - protein, 444 aa, synthetic construct
EVQLVESGGGLVKPGGSLRLSCAASGFTFSNAWLSWVRQAPGKGLEWVGRIKSKTDGGTTDYAAPVKGRFTISRDDSKNTLYLQMNSLKTEDTAVYYCTTGTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 10 aa, synthetic construct
GFTFSNAWLS

SEQ ID NO: 25 - protein, 19 aa, synthetic construct
RIKSKTDGGTTDYAAPVKG

SEQ ID NO: 26 - skipped

SEQ ID NO: 27 - protein, 114 aa, synthetic construct
EVQLVESGGGLVKPGGSLRLSCAASGFTFSNAWLSWVRQAPGKGLEWVGRIKSKTDGGTTDYAAPVKGRFTISRDDSKNTLYLQMNSLKTEDTAVYYCTTGTYWGQGTLVTVSS

SEQ ID NO: 28 - protein, 11 aa, synthetic construct
WGQGTLVTVSS

SEQ ID NO: 29 - protein, 217 aa, synthetic construct
QSALTQPASVSGSPGQSITISCTGTSSDVGSYNLVSWYQQHPGKAPKLMIYEVSKRPSGVSDRFSGSKSANTASLTISGLQAEDEADYYCSSFSINGPSFVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 14 aa, synthetic construct
TGTSSDVGSYNLVS

SEQ ID NO: 31 - protein, 7 aa, synthetic construct
EVSKRPS

SEQ ID NO: 32 - protein, 11 aa, synthetic construct
SSFSINGPSFV

SEQ ID NO: 33 - protein, 111 aa, synthetic construct
QSALTQPASVSGSPGQSITISCTGTSSDVGSYNLVSWYQQHPGKAPKLMIYEVSKRPSGVSDRFSGSKSANTASLTISGLQAEDEADYYCSSFSINGPSFVFGTGTKVTVL

SEQ ID NO: 34 - protein, 10 aa, synthetic construct
FGTGTKVTVL

SEQ ID NO: 35 - protein, 454 aa, synthetic construct
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARVQLDLSYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 10 aa, synthetic construct
GGTFSSYAIS

SEQ ID NO: 37 - protein, 17 aa, synthetic construct
GIIPIFGTANYAQKFQG

SEQ ID NO: 38 - protein, 15 aa, synthetic construct
VQLDLSYYYYYGMDV

SEQ ID NO: 39 - protein, 124 aa, synthetic construct
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARVQLDLSYYYYYGMDVWGQGTTVTVSS

SEQ ID NO: 40 - protein, 217 aa, synthetic construct
NFMLTQPHSVSESPGKTVTISCTGSSGSIASNYVQWYQQRPGSAPTTVIYEDKQRPSGVPDRFSGSIDRSSNSASLTISGLKTEDEADYYCQSYDNSLSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 7 aa, synthetic construct
EDKQRPS

SEQ ID NO: 42 - protein, 10 aa, synthetic construct
QSYDNSLSVV

SEQ ID NO: 43 - protein, 111 aa, synthetic construct
NFMLTQPHSVSESPGKTVTISCTGSSGSIASNYVQWYQQRPGSAPTTVIYEDKQRPSGVPDRFSGSIDRSSNSASLTISGLKTEDEADYYCQSYDNSLSVVFGGGTKLTVL

SEQ ID NO: 44 - protein, 445 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 10 aa, synthetic construct
GYTFTSYGIS

SEQ ID NO: 46 - protein, 17 aa, synthetic construct
WISAYNGNTNYAQKLQG

SEQ ID NO: 47 - protein, 6 aa, synthetic construct
AYGMDV

SEQ ID NO: 48 - protein, 115 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAYGMDVWGQGTTVTVSS

SEQ ID NO: 49 - protein, 215 aa, synthetic construct
QSVLTQPPSASGTPGQRVTISCSVTNSDIDIFNVHWYQQLPGTAPKLLFFHDYRPSGVSDRFSGSKSGTSASLAISGLRSEDEADYYCAGLDVAMRGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 13 aa, synthetic construct
SVTNSDIDIFNVH

SEQ ID NO: 51 - protein, 109 aa, synthetic construct
QSVLTQPPSASGTPGQRVTISCSVTNSDIDIFNVHWYQQLPGTAPKLLFFHDYRPSGVSDRFSGSKSGTSASLAISGLRSEDEADYYCAGLDVAMRGPVFGGGTKLTVL

SEQ ID NO: 52 - protein, 444 aa, synthetic construct
EVQLVESGGGLVQPGGSLRLSCAASGFTFSNAWMSWVRQAPGKGLEWVGRIKSKTDSGTTDYAAPVKGRFTISRDNAKNSLYLQMNSLRAEDTAVYYCTTGTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 10 aa, synthetic construct
GFTFSNAWMS

SEQ ID NO: 54 - protein, 19 aa, synthetic construct
RIKSKTDSGTTDYAAPVKG

SEQ ID NO: 55 - protein, 114 aa, synthetic construct
EVQLVESGGGLVQPGGSLRLSCAASGFTFSNAWMSWVRQAPGKGLEWVGRIKSKTDSGTTDYAAPVKGRFTISRDNAKNSLYLQMNSLRAEDTAVYYCTTGTYWGQGTLVTVSS

SEQ ID NO: 56 - protein, 217 aa, synthetic construct
NFMLTQPHSVSESPGKTVTISCTGSSGNIASNYVQWYQQRPGSAPTTVIYEDKQRPSGVPDRFSGSIDRSSNSASLTISGLKTEDEADYYCQSYDNHLSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 57 - protein, 13 aa, synthetic construct
TGSSGNIASNYVQ

SEQ ID NO: 58 - protein, 10 aa, synthetic construct
QSYDNHLSVV

SEQ ID NO: 59 - protein, 111 aa, synthetic construct
NFMLTQPHSVSESPGKTVTISCTGSSGNIASNYVQWYQQRPGSAPTTVIYEDKQRPSGVPDRFSGSIDRSSNSASLTISGLKTEDEADYYCQSYDNHLSVVFGGGTKLTVL

SEQ ID NO: 60 - protein, 445 aa, synthetic construct
QVQLVQSGAEVKKPGASVKVSCKASGYTFTSYGISWVRQAPGQGLEWMGWISAYIGNTNYAQKLQGRVTMTTDTSISTAYMELSRLRSDDTAVYYCARAYYMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 17 aa, synthetic construct
WISAYIGNTNYAQKLQG

SEQ ID NO: 62 - protein, 6 aa, synthetic construct
AYYMDV

SEQ ID NO: 63 - protein, 115 aa, synthetic construct
QVQLVQSGAEVKKPGASVKVSCKASGYTFTSYGISWVRQAPGQGLEWMGWISAYIGNTNYAQKLQGRVTMTTDTSISTAYMELSRLRSDDTAVYYCARAYYMDVWGQGTTVTVSS

SEQ ID NO: 64 - protein, 215 aa, synthetic construct
QSVLTQPPSASGTPGQRVTISCSVTNPDIDIFNVHWYQQLPGTAPKLLFFHEYRPSGVSDRFSGSKSGTSASLAIVGLQSEDEGYYYCAGLDEAMRGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 13 aa, synthetic construct
SVTNPDIDIFNVH

SEQ ID NO: 66 - protein, 6 aa, synthetic construct
HEYRPS

SEQ ID NO: 67 - protein, 11 aa, synthetic construct
AGLDEAMRGPV

SEQ ID NO: 68 - protein, 109 aa, synthetic construct
QSVLTQPPSASGTPGQRVTISCSVTNPDIDIFNVHWYQQLPGTAPKLLFFHEYRPSGVSDRFSGSKSGTSASLAIVGLQSEDEGYYYCAGLDEAMRGPVFGGGTKLTVL

SEQ ID NO: 69 - protein, 444 aa, synthetic construct
EVQLVESGGGLVKPGGSLRLSCAASGFTFSNAWMSWVRQAPGKGLEWVGRIKSKTDSGTTDYAAPVEGRFTISRDDSKNTLYLQMNSLKTEDTAVYYCTTGTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 19 aa, synthetic construct
RIKSKTDSGTTDYAAPVEG

SEQ ID NO: 71 - protein, 114 aa, synthetic construct
EVQLVESGGGLVKPGGSLRLSCAASGFTFSNAWMSWVRQAPGKGLEWVGRIKSKTDSGTTDYAAPVEGRFTISRDDSKNTLYLQMNSLKTEDTAVYYCTTGTYWGQGTLVTVSS

SEQ ID NO: 72 - protein, 215 aa, synthetic construct
QSVLTQPPSASGTPGQRVTISCSVTNTDIDIFNVHWYQQLPGTAPKLLFFHDYRPSGVSDRFSGSKSGTSASLAIVGLQSEDEGYYYCAGLDEAMRGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - protein, 13 aa, synthetic construct
SVTNTDIDIFNVH

SEQ ID NO: 74 - protein, 109 aa, synthetic construct
QSVLTQPPSASGTPGQRVTISCSVTNTDIDIFNVHWYQQLPGTAPKLLFFHDYRPSGVSDRFSGSKSGTSASLAIVGLQSEDEGYYYCAGLDEAMRGPVFGGGTKLTVL

SEQ ID NO: 75 - protein, 444 aa, synthetic construct
EVQLVESGGGLVKPGGSLRLSCAASGFTFSNAWMSWVRQAPGKGLEWVGRIKSKTEGGDTDYAAPVKGRFTISRDDSKNTLYLQMNSLKTEDTAVYYCTTGTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 19 aa, synthetic construct
RIKSKTEGGDTDYAAPVKG

SEQ ID NO: 77 - protein, 114 aa, synthetic construct
EVQLVESGGGLVKPGGSLRLSCAASGFTFSNAWMSWVRQAPGKGLEWVGRIKSKTEGGDTDYAAPVKGRFTISRDDSKNTLYLQMNSLKTEDTAVYYCTTGTYWGQGTLVTVSS

SEQ ID NO: 78 - protein, 429 aa, Homo sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 79 - protein, 110 aa, Homo sapiens
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 80 - protein, 10 aa, synthetic construct
GFTFSNAWXS

SEQ ID NO: 81 - protein, 19 aa, synthetic construct
RIKSKTXXGXTDYAAPVXG

SEQ ID NO: 82 - protein, 13 aa, synthetic construct
SVTNXDIDIXNVH

SEQ ID NO: 83 - protein, 6 aa, synthetic construct
HXYRPS

SEQ ID NO: 84 - protein, 11 aa, synthetic construct
AGLDXAMRGPV

SEQ ID NO: 85 - DNA, 342 bp, synthetic construct
  source 1..342: mol_type = other DNA
gaggtgcagctggtggagtctgggggaggcttggtaaagcctggggggtcccttagactctcctgtgcagcctctggattcacttttagtaacgcctggatgagctgggtccgccaggctccagggaaggggctggagtgggttggccgtattaaaagcaaaactgagggtggggatacagactacgctgcacccgtgaaaggcagattcaccatctcaagagatgattcaaaaaacacgctgtatctgcaaatgaacagcctgaaaaccgaggacacagccgtgtattactgtaccacaggtacttactggggccagggaaccctggtcaccgtctcgagc

SEQ ID NO: 86 - DNA, 327 bp, synthetic construct
  source 1..327: mol_type = other DNA
cagtctgtgctgactcagccaccctcagcgtctgggacccccggccagagggtcactatttcttgctctgtcaccaacaccgacatcgacattttcaatgttcactggtatcagcagctcccaggaacggcccccaaactcctcttctttcatgattaccggccctcaggggtctctgaccgattttccggctccaagtctggaacctcagcctccctggccatcgttggcctccagtctgaggatgagggttattattactgtgcaggattggatgaggctatgaggggtccggttttcggcggcgggaccaagctgaccgtccta

SEQ ID NO: 87 - protein, 1332 aa, synthetic construct
GAGGTGCAGCTGGTGGAGTCTGGGGGAGGCTTGGTAAAGCCTGGGGGGTCCCTTAGACTCTCCTGTGCAGCCTCTGGATTCACTTTTAGTAACGCCTGGATGAGCTGGGTCCGCCAGGCTCCAGGGAAGGGGCTGGAGTGGGTTGGCCGTATTAAAAGCAAAACTGAGGGTGGGGATACAGACTACGCTGCACCCGTGAAAGGCAGATTCACCATCTCAAGAGATGATTCAAAAAACACGCTGTATCTGCAAATGAACAGCCTGAAAACCGAGGACACAGCCGTGTATTACTGTACCACAGGTACTTACTGGGGCCAGGGAACCCTGGTCACCGTCTCGAGCGCGTCGACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAAAGTTGAGCCCAAATCTTGTGACAAAACTCACACATGCCCACCGTGCCCAGCACCTGAAGCCGCTGGGGCACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGAGGAGATGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCCCCGGGAAAA

SEQ ID NO: 88 - DNA, 645 bp, synthetic construct
  source 1..645: mol_type = other DNA
cagtctgtgctgactcagccaccctcagcgtctgggacccccggccagagggtcactatttcttgctctgtcaccaacaccgacatcgacattttcaatgttcactggtatcagcagctcccaggaacggcccccaaactcctcttctttcatgattaccggccctcaggggtctctgaccgattttccggctccaagtctggaacctcagcctccctggccatcgttggcctccagtctgaggatgagggttattattactgtgcaggattggatgaggctatgaggggtccggttttcggcggcgggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 89 - protein, 19 aa, synthetic construct
MGWSCIILFLVATATGVHS